(19) United States
(12) Patent Application Publication
(10) Pub. No.: US 2024/0270705 A1
(43) Pub. Date: Aug. 15, 2024

(54) COMPOUNDS SELECTIVE FOR JAK1 AND METHODS OF USE

(71) Applicant: Vividion Therapeutics, Inc., San Diego, CA (US)

(72) Inventors: Benjamin HORNING, San Diego, CA (US); Nilotpal ROY, San Diego, CA (US); Matthew PATRICELLI, San Diego, CA (US); Betty LAM, San Diego, CA (US); Shota KIKUCHI, San Diego, CA (US); Brian Nicholas COOK, San Diego, CA (US)

(21) Appl. No.: 18/563,329

(22) PCT Filed: May 25, 2022
(86) PCT No.: PCT/US2022/030811

§ 371 Date: Nov. 21, 2023

Related U.S. Application Data
(60) Provisional application No. 63/192,908, filed on May 25, 2021.

(51) Int. Cl. C07D 265/30; A61K 31/40; A61K 31/444; A61K 31/4525; A61K 31/454; A61K 31/4545; A61K 31/506; A61K 31/5375; A61K 31/55; C07D 207/08; C07D 211/18; C07D 211/38; C07D 211/42; C07D 211/44; C07D 223/04; C07D 241/04; C07D 401/04; C07D 401/06; C07D 405/06; C12N 9/12

(52) U.S. Cl. CPC C07D 265/30; A61K 31/40; A61K 31/444; A61K 31/4525; A61K 31/454; A61K 31/4545; A61K 31/506; A61K 31/5375; A61K 31/55; C07D 207/08; C07D 211/18; C07D 211/38; C07D 211/42; C07D 211/44; C07D 223/04; C07D 241/04; C07D 401/04; C07D 401/06; C07D 405/06; C12N 9/1205

(57) ABSTRACT

Disclosed herein are compounds selective for JAK1, pharmaceutical compositions comprising said compounds, and methods of using said compounds.

### CROSS REFERENCE

This application claims the benefit of U.S. Application No. 63/192,908, filed May 25, 2021, which is hereby incorporated by reference in its entirety.

### SEQUENCE LISTING

present application is being filed along with a Sequence Listing in electronic format. The Sequence Listing is provided as a file entitled “48054_729_601_sequence_listing,” created May 12, 2022, which is 39.1 kilobytes in size. The information in the electronic format of the Sequence Listing is incorporated by reference in its entirety.

### BACKGROUND OF THE DISCLOSURE

Protein kinases are enzymes that catalyze the phosphorylation of specific residues in proteins, and are broadly classified into tyrosine and serine/threonine kinases. Inappropriate kinase activity, arising from mutation, over-expression, or inappropriate regulation, dysregulation or de-regulation, as well as over- or under-production of growth factors or cytokines has been implicated in many diseases, including but not limited to cancer, cardiovascular diseases, allergies, asthma and other respiratory diseases, autoimmune diseases, inflammatory diseases, bone diseases, metabolic disorders, and neurological and neurodegenerative disorders.

The JAK (Janus-associated kinase) family includes four non-receptor tyrosine kinases, JAK1, JAK2, JAK3 and Tyk2, which play a critical role in cytokine signaling and growth factor mediated signal transduction. Upon binding to their receptors, cytokines activate JAK, which then phosphorylates the cytokine receptor, thereby creating docking sites for signaling molecules, notably, members of the Signal Transducer and Activator of Transcription (STAT) family that ultimately lead to gene expression. Numerous cytokines are known to activate the JAK family.

JAK1 kinase interacts with, among others, type I interferon receptors (e.g., IFN alpha), type II interferon receptors (e.g., IFN gamma), the common gamma chain ye (e.g., IL-2, IL-4, IL-7, IL-9, IL-15 and IL-21), and the interleukin-6 family (IL-10, IL-13 and IL-22). After these cytokines bind to their receptors, receptor oligomerization occurs, resulting in the cytoplasmic tails of associated JAK kinases moving into proximity and facilitating the trans-phosphorylation and activation of tyrosine residues on the JAK kinase. Phosphorylated JAK kinases bind and activate various STAT proteins. These STAT proteins then dimerize and translocate to the nucleus where they function as both signaling molecules and transcription factors and ultimately bind to specific DNA sequences present in the promoters of cytokine-responsive genes. Various immunodeficiency and autoimmune diseases, such as allergies, asthma, alopecia areata, transplant (allograft) rejection, rheumatoid arthritis, amyotrophic lateral sclerosis and multiple sclerosis, and solid and hematologic cancers result from signaling disruption in the JAK/STAT pathway.

An important element of JAK1 is the ability to pair with other JAK kinases at the intracellular domains of different subunits of the receptor. It has been indicated that selective inhibition of JAK1 may be sufficient to treat a number of inflammatory and autoimmune diseases associated with cytokine signaling via the JAK-STAT pathway.

There exists a need for inhibitors of the JAK-STAT signaling pathway and for treatment of JAK1-related disorders, including allergic, inflammatory, and autoimmune disorders.

### SUMMARY OF THE DISCLOSURE

In one aspect, disclosed herein is a compound of Formula (II), or a pharmaceutically acceptable salt or solvate thereof:

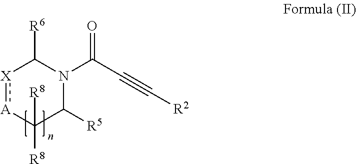

wherein:
   [symbol] is a single or double bond;
   X is C(J)(R⁷) when [symbol] is a single bond and C(J) when [symbol] is a double bond;
   A is CR₂, NR¹, O or S or S(═O)₂ when [symbol] is a single bond; and CR when [symbol] is a double bond;
each R independently is H, halo, or C₁-C₆ alkyl;
R¹ is selected from the group C₁-C₆ alkyl, S(═O)₂—(C₁-C₆)alkyl, S(═O)₂—(C₆-C₁₀) aryl, C(═O)—(C₁-C₆) alkyl, or C(═O)—(C₆-C₁₀)aryl;
J is a C₆-C₁₀ aryl or a five- to six-membered heteroaryl, each of which is optionally substituted with 1-4 R³;
each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halo, C₁-C₆ haloalkyl, cyano and N(R⁴)₂; or two R³ optionally are taken together with the carbon atoms to which they are attached to form a five- or six-membered heterocycle or a C₃-C₆ cycloalkyl;
R² is selected from the group consisting of C₆-C₁₀ aryl, five- or six-membered heteroaryl, C₁-C₆ alkyl, C₃-C₈ cycloalkyl, C₁-C₆ alkyl-hydroxy, C₁-C₆ alkoxy, C₁-C₆ alkyl-amino, C₂-C₆ alkenyl, C₂-C₆ alkynyl, C₁-C₆ haloalkyl, or four- to six-membered heterocyclyl, each of which is optionally substituted with 1-3 R⁴;
each R⁴ is independently selected from the group consisting of hydroxyl, C₁-C₆ alkoxy, C₁-C₆ alkyl, C(═O)R^{4a} or S(═O)₂R^{4a} or wherein two R⁴ optionally are taken together with the carbon atoms to which they are attached to form a five- to six-membered heterocyclyl or a C₃-C₆ cycloalkyl, wherein each R^{4a} independently is hydrogen or C₁-C₆ alkyl;
R⁵ is selected from the group consisting of hydrogen, C₁-C₆ alkyl, hydroxy, C₁-C₆ alkoxy, halogen, and —N(R³)₂;
R⁶ is H or C₁-C₆ alkyl;
R⁷ is H, hydroxy, or C₁-C₆ alkyl;
each R⁸ independently is H, halo, C₁-C₆ alkoxy, or C₁-C₆ alkyl; and
n is 0, 1, or 2;
or a compound selected from the group consisting of

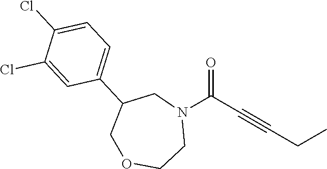 [SMILES: CCC#CC(=O)N1CCOCC(c2ccc(Cl)c(Cl)c2)C1]

or a pharmaceutically acceptable salt or solvate thereof.

In some embodiments, for the compound of Formula (II) or the pharmaceutically acceptable salt or solvate thereof,
in J:
the optionally substituted C₆-C₁₀ aryl is phenyl which is optionally substituted with 1-4 R³;
the optionally substituted five- to six-membered heteroaryl is selected from the group consisting of pyridyl, pyrazolyl, and pyrimidinyl, each of which is optionally substituted with 1-4 R³;
in R¹:
the C₁-C₆ alkyl is methyl;
the C₁-C₆ alkyl in S(═O)₂—(C₁-C₆)alkyl is methyl;
the C₆-C₁₀ aryl in S(═O)₂—(C₆-C₁₀) aryl is phenyl;
the C₁-C₆ alkyl in C(═O)—(C₁-C₆) alkyl is methyl; and
the C₆-C₁₀ aryl in C(═O)—(C₆-C₁₀) aryl is phenyl;
in R³:
the C₁-C₆ alkyl is selected from the group consisting of methyl, and ethyl;
the C₃-C₆ cycloalkyl is cyclopropyl;
the C₁-C₆ alkoxy is methoxy; and
the C₁-C₆ haloalkyl is trifluoromethyl;
in R²:
the four- to six-membered heterocyclyl is selected from the group consisting of azetidinyl, tetrahydrofuranyl, piperidinyl, and pyrrolidinyl, each of which is optionally substituted with 1-3 R⁴;
the C₆-C₁₀ aryl is phenyl which is optionally substituted with 1-3 R⁴;
the C₃-C₈ cycloalkyl is selected from the group consisting of cyclopropyl, cyclobutyl, and cyclopentyl, each of which is optionally substituted with 1-3 R⁴;
the five- to six-membered heteroaryl is pyrazolyl which is optionally substituted with 1-3 R⁴;
the C₁-C₆ alkyl is selected from the group consisting of methyl, ethyl, and isopropyl, which is optionally substituted with 1-3 R⁴;
the C₁-C₆ alkyl-hydroxy is hydroxymethyl; and
the C₁-C₆ alkyl-amino is —CH₂—CH₂—NH—;
in R⁴:
the R^{4a} in S(═O)₂R^{4a} is selected from the group consisting of methyl and phenyl; and
the R^{4a} in C(═O)R^{4a} is methyl;
in R⁴:
the C₁-C₆ alkyl is methyl;
in R⁶:
the C₁-C₆ alkyl is methyl; and
in R⁸:
the C₁-C₆ alkyl is methyl; and
the C₁-C₆ alkoxy is methoxy.

In another aspect, disclosed herein is a compound of Formula (II), or a pharmaceutically acceptable salt or solvate thereof:

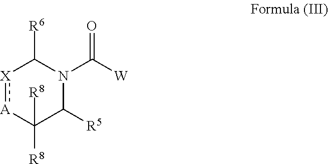

wherein:
   [symbol] is a single or double bond;
   X is C(J)(R⁷) when [symbol] is a single bond and C(J) when [symbol] is a double bond;
A is CR², NR¹, O or S or S(═O)₂ when [symbol] is a single bond; and CR when [symbol] is a double bond;
each R independently is H, halo, or C₁-C₆ alkyl;
R¹ is selected from the group C₁-C₆ alkyl, S(═O)₂—(C₁-C₆)alkyl, S(═O)₂—(C₆-C₁₀) aryl, C(═O)—(C₁-C₆) alkyl, or C(═O)—(C₆-C₁₀)aryl;
J is a C₆-C₁₀ aryl or a five- to six-membered heteroaryl, each of which is optionally substituted with 1-4 R³;
each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halo, C₁-C₆ haloalkyl, cyano and N(R⁴)₂; or two R optionally are taken together with the carbon atoms to which they are attached to form a five- or six-membered heterocycle or a C₃-C₆ cycloalkyl;
R⁵ is selected from the group consisting of hydrogen, C₁-C₆ alkyl, hydroxy, C₁-C₆ alkoxy, halogen, and —N(R³)₂;
R⁶ is H or C₁-C₆ alkyl;
R⁷ is H, hydroxy, or C₁-C₆ alkyl;
each R⁸ independently is H, halo, C₁-C₆ alkoxy, or C₁-C₆ alkyl;
n is 0, 1, or 2; and
W comprises an electrophile that reacts and forms a covalent bond with the sulfur atom at cysteine 817 as set forth in SEQ ID NO: 1, 2, 3, or 4.

In another aspect, provided herein is a pharmaceutical composition comprising a compound of Formula (I), (II), or (III), or a pharmaceutically acceptable salt or solvate thereof; and a pharmaceutically acceptable excipient or carrier. In some embodiments, the pharmaceutical composition comprises a second therapeutic agent.

In another aspect, provided herein is a method of inhibiting JAK in a subject, comprising administering to the subject in need of such inhibition a therapeutically effective amount of a compound of Formula (I), (II), or (III), or a pharmaceutically acceptable salt or solvate thereof.

In another aspect, disclosed herein is a method of treating a disease mediated by JAK1 in a subject, comprising administering to the subject in need of such treatment a therapeutically effective amount of a compound of Formula (I), (II), or (III), or a pharmaceutically acceptable salt or solvate thereof. In some embodiments, the disease is an inflammatory or autoimmune disease. In some embodiments, the inflammatory or autoimmune disease is selected from the group consisting of Rheumatoid Arthritis (RA), Crohn's disease, ankylosing spondylitis (AS), psoriatic arthritis, psoriasis, ulcerative colitis, systemic lupus erytheinatosus (SLE), diabetic nephropathy, dry eye syndrome, Sjogren's Syndrome, alopecia areata, vitiligo, and atopic dermatitis.

In another aspect, provided herein is a method of inhibiting JAK1 comprising effecting a non-naturally occurring covalent modification at cysteine 817 as set forth in SEQ ID NO: 1, 2, 3, or 4, the modification resulting from a bond forming reaction between an electrophile and the cysteine 817 as set forth in SEQ ID NO: 1, 2, 3, or 4, wherein a sulfur atom at the cysteine residue undergoes a reaction with the electrophile.

In another aspect, disclosed herein is a modified JAK1 protein comprising a non-naturally occurring small molecule fragment having a covalent bond to cysteine 817 of the JAK1 protein, wherein the modified JAK1 protein comprises SEQ ID NO: 1, 2, 3, or 4; and has the structure of Formula (IV):

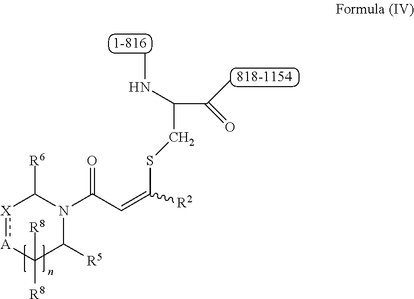

wherein in Formula (IV):
[symbol] is a single or double bond;
X is C(J)(R⁷) when [symbol] is a single bond and C(J) when [symbol] is a double bond;
A is CR², NR¹, O or S or S(═O)₂ when [symbol] is a single bond; and CR when [symbol] is a double bond;
each R independently is H, halo, or C₁-C₆ alkyl;
R¹ is selected from the group C₁-C₆ alkyl, S(═O)₂—(C₁-C₆)alkyl, S(═O)₂—(C₆-C₁₀) aryl, C(═O)—(C₁-C₆) alkyl, or C(═O)—(C₆-C₁₀)aryl;
J is a C₆-C₁₀ aryl or a five- to six-membered heteroaryl, each of which is optionally substituted with 1-4 R³
each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halo, C₁-C₆ haloalkyl, cyano and N(R⁴)₂; or two R³ optionally are taken together with the carbon atoms to which they are attached to form a five- or six-membered heterocycle or a C₃-C₆ cycloalkyl;
R² is selected from the group consisting of C₆-C₁₀ aryl, five- or six-membered heteroaryl, C₁-C₆ alkyl, C₃-C₈ cycloalkyl, C₁-C₆ alkyl-hydroxy, C₁-C₆ alkoxy, C₁-C₆ alkyl-amino, C₂-C₆ alkenyl, C₂-C₆ alkynyl, C₁-C₆ haloalkyl, or four- to six-membered heterocyclyl, each of which is optionally substituted with 1-3 R⁴;
each R⁴ is independently selected from the group consisting of hydroxyl, C₁-C₆ alkoxy, C₁-C₆ alkyl, C(═O)R^{4a} or S(═O)₂R^{4a} or wherein two R⁴ optionally are taken together with the carbon atoms to which they are attached to form a five- to six-membered heterocyclyl or a C₃-C₆ cycloalkyl, wherein each R^{4a} independently is hydrogen or C₁-C₆ alkyl;
R⁵ is selected from the group consisting of hydrogen, C₁-C₆ alkyl, hydroxy, C₁-C₆ alkoxy, halogen, and —N(R³)₂;
R⁶ is H or C₁-C₆ alkyl;
R⁷ is H, hydroxy, or C₁-C₆ alkyl;
each R⁸ independently is H, halo, C₁-C₆ alkoxy, or C₁-C₆ alkyl; and
n is 0, 1, or 2; and

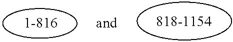

and represent amino acid sequences 1-816 and 818-1154 respectively in SEQ ID NO: 1, 2, 3, or 4.

In another aspect, provided herein is a JAK1 binding domain wherein said binding domain comprises a cysteine at position 817 of JAK1, wherein the numbering of the amino acid positions corresponds to position 817 in SEQ ID NO: 1, 2, 3, or 4. In some embodiments the present invention provides a neoprotein comprising JAK1 protein connected through a covalent bond to a compound of Formula (I), (II), or (III) through the cysteine residue C817 of JAK1, wherein the numbering of the amino acid positions corresponds to the amino acid positions in SEQ ID NO: 1, 2, 3, or 4.

In some embodiments, provided herein are adducts of a compound of Formula (I), (II), or (III), or a pharmaceutically acceptable salt or solvate thereof and JAK1. In some embodiments, adducts of the compounds described herein modulate binding in JAK1 protein. In some embodiments, provided herein are adducts of a compound of Formula (I), (II), or (III) and JAK1 that modulate pseudokinase domain (JH2) binding in a JAK1 protein. In some embodiments, provided herein are adducts of a compound of Formula (I), (II), or (III) and JAK1 that modulate JAK1-related cytokine signaling pathways through allosteric inhibition of JAK1 binding to homo- or heterodimerization partners. In some instances, also provided herein are JAK1 ligands that bind to a cysteine in the JH2 domain. In some embodiments, binding to a compound of Formula (I), (II), or (III) modulates the crystal structure of JAK1. In some embodiments, binding to a compound of Formula (I), (II), or (III) does not inhibit JAK1 binding ATP.

In another aspect, provided herein is a method of selectively inhibiting JAK1 over TYK2. In some embodiments, the compound disclosed herein is more than 2-fold selective over TYK2. In some embodiments, the compound disclosed herein is more than 5-fold selective over TYK2. In some embodiments, the compound disclosed herein is more than 8-fold selective over TYK2. In some embodiments, the compound disclosed herein is more than 10-fold selective over TYK2. In some embodiments, the compound disclosed herein is more than 20-fold selective over TYK2. In some embodiments, the compound disclosed herein is more than 50-fold selective over TYK2. In some embodiments, the compound disclosed is more than 100-fold selective over TYK2.

In another aspect, provided herein is a method of selectively inhibiting JAK1 over one or more of JAK2 and JAK3. In some embodiments, the compound disclosed herein is more than 2-fold selective over JAK2/3. In some embodiments, the compound disclosed herein is more than 5-fold selective over JAK2/3. In some embodiments, the compound disclosed herein is more than 10-fold selective over JAK2/3. In some embodiments, the compound disclosed herein is more than 20-fold selective over JAK2/3. In some embodiments, the compound disclosed herein is more than 50-fold selective over JAK2/3. In some embodiments, the compound disclosed herein is more than 100-fold selective over JAK2/3.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 displays the JH2 domain of JAK1, which contains cysteine 817. Prolines at positions 733 and 832 are shown. Prolines 733 and 832 of SEQ ID NO: 1 are mutated to leucine and serine, respectively, in a JAK1-impaired patient.

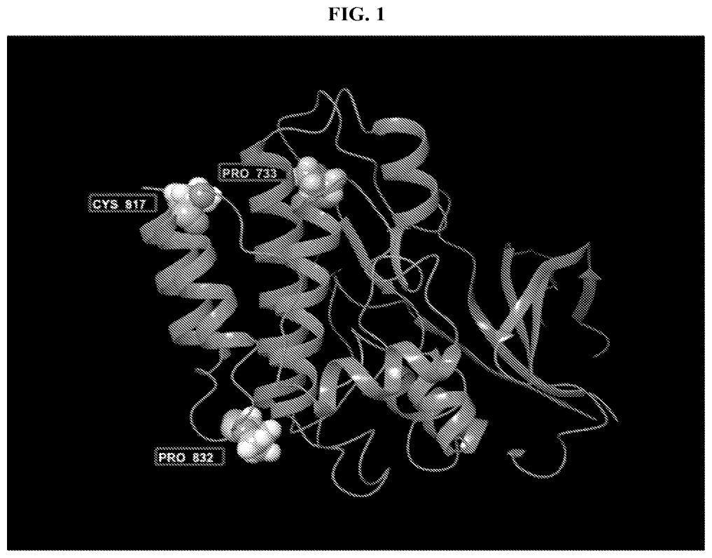

### DETAILED DESCRIPTION OF THE DISCLOSURE

Protein kinases are a broad and diverse class, of over 500 enzymes, that include oncogenes, growth factors receptors, signal transduction intermediates, apoptosis related kinases and cyclin dependent kinases. They are responsible for the transfer of a phosphate group to specific tyrosine, serine or threonine amino acid residues, and are broadly classified as tyrosine and scrine/threonine kinases as a result of their substrate specificity.

The Janus family kinases (JAK1, JAK2, JAK3 and TYK2) are cytoplasmic tyrosine kinases that associate with membrane bound cytokine receptors. Cytokine binding to their receptor initiates Janus kinase activation via trans and autophosphorylation processes. The activated JAK phosphorylates residues on the cytokine receptors creating phosphotyrosine binding sites for SH2 domain containing proteins such as Signal Transduction Activators of Transcript (STAT) factors and other signal regulators transduction such as suppressor of cytokine signaling (SOCS) proteins and SH2 domain-containing inositol 5′-phosphatases (SHIP). Activation of STAT factors via this process leads to their dimerization, nuclear translocation and new mRNA transcription resulting in expression of immunocyte proliferation and survival factors as well as additional cytokines, chemokines and molecules that facilitate cellular trafficking (see Journal of Immunology, 2007, 178, p. 2623). Jak kinases transduce signals for many different cytokine families and hence potentially play roles in diseases with widely different pathologies including but not limited to the following examples. Both JAK1 and JAK3 control signaling of the so-called common gamma chain cytokines (IL2, IL4, IL7, IL9, IL15 and IL21), hence simultaneous inhibition of either JAK1 or JAK3 could be predicted to impact Th1 mediated diseases such as rheumatoid arthritis via blockade of IL2, IL7 and IL15 signaling. On the other hand, IL2 signaling has recently been shown to be essential for development and homeostasis of T-regulatory cells (Malek T R et al., Immunity, 2002, 17(2), p. 167-78). Thus, based on genetic data, blockade of IL2 signaling alone is predicted to result in autoimmunity (Yamanouchi J et al., Nat Genet., 2007, 39(3), p. 329-37, and Willerford D M et al., Immunity, 1995, 3(4), p. 521-30). Th2 mediated diseases such as asthma or atopic dermatitis via IL4 and IL9 signaling blockade. JAK1 and TYK2 mediate signaling of IL13 (see Int. Immunity, 2000, 12, p. 1499). Hence, blockade of these may also be predicted to have a therapeutic effect in asthma. These two kinases are also thought to mediate Type I interferon signaling; their blockade could therefore be predicted to reduce the severity of systemic lupus erythematosus (SLE). TYK2 and JAK2 mediate signaling of IL12 and IL23. In fact, blockade of these cytokines using monoclonal antibodies has been effective in treating psoriasis.

Allosteric kinase inhibitors (type III and type IV, Pharmacol Res., 2016, 103, p. 26) offer many potential advantages over ATP competitive kinase inhibitors (Angew Chem Int Ed Engl, 2020, 59(33), p. 13764). Conservation of protein sequences surrounding the kinase active site can lead to selectivity challenges and poor cellular efficacy due to competition with endogenous ATP. In one embodiment, compounds of the present invention allosterically inhibit JAK1 through C817. In some embodiments the present invention provides methods of inhibiting JAK1 with one or more non-synonymous single-nucleotide polymorphisms [nsSNPs, ˜0.9% human JAK1, Pharmacol Res., 2016, 103, p. 26] comprising administering a compound of the present invention.

Several pathologically significant cytokines signal via JAK1 alone (Guschin D, et al., EMBO J. 1995 Apr. 3; 14(7):1421-9; Parganas E, et al., Cell. 1998 May 1; 93(3):385-95; Rodig S. J., et al., Cell. 1998 May 1; 93(3):373-83). Blockade of one of these, IL6, using an IL6R neutralizing antibody, has been shown to significantly improve disease scores in human rheumatoid arthritis patients (Nishimoto N. et al., Ann Rheum Dis., 2007, 66(9), p. 1162-7). Similarly, blockaded of GCSF signaling, which is also mediated by Jak1 alone, using neutralizing monoclonal antibodies or target gene deletion protects mice from experimental arthritis (Lawlor K. E. et al., Proc Natl Acad Sci U.S.A., 2004, 101(31), p. 11398-403). Accordingly, the identification of small-molecule compounds that inhibit, regulate and/or modulate the signal transduction of kinases, such as JAK1, is a desirable means to prevent or treat autoimmune diseases or other diseases related to aberrant JAK1 function.

JAK2 is also activated in a wide variety of human cancers such as prostate, colon, ovarian and breast cancers, melanoma, leukemia and other haematopoietic malignancies. In addition, somatic point mutation of the JAK2 gene has been identified to be highly associated with classic myeloproliferative disorders (MPD) and infrequently in other myeloid disorders. Constitutive activation of JAK2 activity is also caused by chromosomal translocation in hematopoeitic malignancies. It has also been shown that inhibition of the JAK/STAT pathway, and in particular inhibition of JAK2 activity, results in anti-proliferative and pro-apoptotic effects largely due to inhibition of phosphorylation of STAT. Furthermore, pharmacological modulation or inhibition of JAK2 activity could effectively block tumor growth and induce apoptosis by reducing the STAT phosphorylation in cell culture and human tumor xenografts in vivo. Accordingly, the identification of small-molecule compounds that inhibit, regulate and/or modulate the signal transduction of kinases, particularly JAK2, is desirable as a means to treat or prevent diseases and conditions associated with cancers.

Jak kinases also transmit signals regulating essential physiological processes whose inhibition could be undesirable. For example, JAK2 mediates the signaling of Erythropoetin (Epo) and Granulocyte/Monocyte-Colony Stimulating Factor (GM-CSF). Individuals with genetic, congenital or acquired defects in these signaling pathways can develop potentially life-threatening complications such as anemia and neutrophil dysfunction. Accordingly, one non-limiting aspect of this invention also relates to a method to identify compounds that may have a favorable safety profile as a result of them selectively avoiding inhibition of JAK2.

The protein kinase C family is a group of serine/threonine kinases that comprises twelve related isoenzymes. Its members are encoded by different genes and are sub-classified according to their requirements for activation. The classical enzymes (cPKC) require diacylglycerol (DAG), phosphatidylserine (PS) and calcium for activation. The novel PKC's (nPKC) require DAG and PS but are calcium independent. The atypical PKC's (aPKC) do not require calcium or DAG.

PKCtheta is a member of the nPKC sub-family (Baier, G., et al., J. Biol. Chem., 1993, 268, 4997). It has a restricted expression pattern, found predominantly in T cells and skeletal muscle (Mischak, H. et al., FEBS Lett., 1993, 326, p. 51), with some expression reported in mast cells (Liu, Y. et al., J. Leukoc. Biol., 2001, 69, p. 831) and endothelial cells (Mattila, P. et al., Life Sci., 1994, 55, p. 1253).

Upon T cell activation, a supramolecular activation complex (SMAC) forms at the site of contact between the T cell and the antigen presenting cell (APC). PKCtheta is the only PKC isoform found to localize at the SMAC (Monks, C. et al., Nature, 1997, 385, 83), placing it in proximity with other signaling enzymes that mediate T cell activation processes.

In another study (Baier-Bitterlich, G. et al., Mol. Cell. Biol., 1996, 16, 842) the role of PKCtheta in the activation of AP-1, a transcription factor important in the activation of the IL-2 gene, was confirmed. In unstimulated T cells, constitutively active PKCtheta stimulated AP-1 activity while in cells with dominant negative PKCtheta, AP-1 activity was not induced upon activation by PMA.

Other studies showed that PKCtheta, via activation of icB kinase beta, mediates activation of NF-icB induced by T cell receptor/CD28 co-stimulation (N. Coudronniere et al., Proc. Nat. Acad. Sci. U.S.A., 2000, 97, p. 3394; and Lin, X. et al., Mol. Cell. Biol., 2000, 20, p. 2933).

Proliferation of peripheral T cells from PKCtheta knockout mice, in response to T cell receptor (TCR)/CD28 stimulation was greatly diminished compared to T cells from wild type mice. In addition, the amount of IL-2 released from the T cells was also greatly reduced (Sun, Z. et al., Nature, 2000, 404, p. 402). It has also been shown that PKCtheta-deficient mice show impaired pulmonary inflammation and airway hyperresponsiveness (AHR) in a Th2-dependent murine asthma model, with no defects in viral clearance and Th1-dependent cytotoxic T cell function (Berg-Brown, N. N. et al., J. Exp. Med., 2004, 199, p. 743; Marsland, B. J. et al., J. Exp. Med., 2004, 200, p. 181). The impaired Th2 cell response results in reduced levels of IL-4 and immunoglobulin E (IgE), contributing to the AHR and inflammatory pathophysiology. Otherwise, the PKCtheta knockout mice seemed normal and fertile.

Evidence exists that PKCtheta participates in the IgE receptor (FceRI)-mediated response of mast cells (Liu, Y. et al., J. Leukoc. Biol., 2001, 69, p. 831). In human-cultured mast cells (HCMC), it has been demonstrated that PKC kinase activity rapidly localizes to the membrane following FceRI cross-linking (Kimata, M. et al., Biochem. Biophys. Res. Commun., 1999, 257(3), p. 895). A recent study examining in vitro activity of bone marrow mast cells (BMMC) derived from wild-type and PKCtheta-deficient mice shows that upon FceRI cross linking, BMMCs from PKCtheta-deficient mice reduced levels of IL-6, tumor necrosis factor-alpha (TNF alpha) and IL-13 in comparison with BMMCs from wild-type mice, suggesting a potential role for PKCtheta in mast cell cytokine production in addition to T cell activation (Ciarletta, A. B. et al., poster presentation at the 2005 American Thoracic Society International Conference).

Many of the kinases, whether a receptor or non-receptor tyrosine kinase or a S/T kinase have been found to be involved in cellular signaling pathways involved in numerous pathogenic conditions, including immunomodulation, inflammation, or proliferative disorders such as cancer.

Many autoimmune diseases and disease associated with chronic inflammation, as well as acute responses, have been linked to excessive or unregulated production or activity of one or more cytokines.

### Compounds

In one aspect disclosed herein is a compound of Formula (II), or a pharmaceutically acceptable salt or solvate thereof:

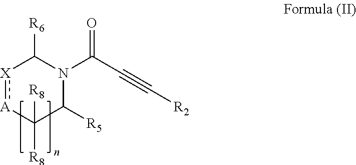

wherein:
   [symbol] is a single or double bond;
   X is C(J)(R⁷) when [symbol] is a single bond and C(J) when [symbol] is a double bond;
A is CR², NR¹, O or S or S(═O)₂ when [symbol] is a single bond; and CR when [symbol] is a double bond;
each R independently is H, halo, or C₁-C₆ alkyl;
R¹ is selected from the group C₁-C₆ alkyl, S(═O)₂—(C₁-C₆)alkyl, S(═O)₂—(C₆-C₁₀) aryl, C(═O)—(C₁-C₆) alkyl, or C(═O)—(C₆-C₁₀)aryl;
J is a C₆-C₁₀ aryl or a five- to six-membered heteroaryl, each of which is optionally substituted with 1-4 R³;
each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halo, C₁-C₆ haloalkyl, cyano and N(R⁴)₂; or two R³ optionally are taken together with the carbon atoms to which they are attached to form a five- or six-membered heterocycle or a C₃-C₆ cycloalkyl;
R² is selected from the group consisting of C₆-C₁₀ aryl, five- or six-membered heteroaryl, C₁-C₆ alkyl, C₃-C₈ cycloalkyl, C₁-C₆ alkyl-hydroxy, C₁-C₆ alkoxy, C₁-C₆ alkyl-amino, C₂-C₆ alkenyl, C₂-C₆ alkynyl, C₁-C₆ haloalkyl, or four- to six-membered heterocyclyl, each of which is optionally substituted with 1-3 R⁴;
each R⁴ is independently selected from the group consisting of hydroxyl, C₁-C₆ alkoxy, C₁-C₆ alkyl, C(═O)R^{4a} or S(═O)₂R^{4a} or wherein two R⁴ optionally are taken together with the carbon atoms to which they are attached to form a five- to six-membered heterocyclyl or a C₃-C₆ cycloalkyl, wherein each R^{4a} independently is hydrogen or C₁-C₆ alkyl;
R⁵ is selected from the group consisting of hydrogen, C₁-C₆ alkyl, hydroxy, C₁-C₆ alkoxy, halogen, and —N(R³)₂;
R⁶ is H or C₁-C₆ alkyl;
R⁷ is H, hydroxy, or C₁-C₆ alkyl;
each R independently is H, halo, C₁-C₆ alkoxy, or C₁-C₆ alkyl; and
n is 0, 1, or 2;
or a compound selected from the group consisting of

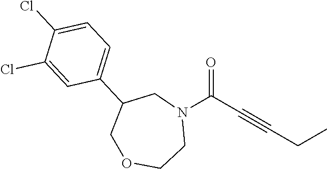 [SMILES: CCC#CC(=O)N1CCOCC(c2ccc(Cl)c(Cl)c2)C1]

or a pharmaceutically acceptable salt or solvate thereof.

In some embodiments, for the compound of Formula (II) or the pharmaceutically acceptable salt or solvate thereof,
in J:
the optionally substituted C₆-C₁₀ aryl is phenyl which is optionally substituted with 1-4 R³;
the optionally substituted five- to six-membered heteroaryl is selected from the group consisting of pyridyl, pyrazolyl, and pyrimidinyl, each of which is optionally substituted with 1-4 R³;
in R¹:
the C₁-C₆ alkyl is methyl;
the C₁-C₆ alkyl in S(═O)₂—(C₁-C₆)alkyl is methyl;
the C₆-C₁₀ aryl in S(═O)₂—(C₆-C₁₀) aryl is phenyl;
the C₁-C₆ alkyl in C(═O)—(C₁-C₆) alkyl is methyl; and
the C₆-C₁₀ aryl in C(═O)—(C₆-C₁₀) aryl is phenyl;
in R³:
the C₁-C₆ alkyl is selected from the group consisting of methyl, and ethyl;
the C₃-C₆ cycloalkyl is cyclopropyl;
the C₁-C₆ alkoxy is methoxy; and
the C₁-C₆ haloalkyl is trifluoromethyl;
in R²:
the four- to six-membered heterocyclyl is selected from the group consisting of azetidinyl, tetrahydrofuranyl, piperidinyl, and pyrrolidinyl, each of which is optionally substituted with 1-3 R⁴;
the C₆-C₁₀ aryl is phenyl which is optionally substituted with 1-3 R⁴;
the C₃-C₈ cycloalkyl is selected from the group consisting of cyclopropyl, cyclobutyl, and cyclopentyl, each of which is optionally substituted with 1-3 R⁴;
the five- to six-membered heteroaryl is pyrazolyl which is optionally substituted with 1-3 R⁴;
the C₁-C₆ alkyl is selected from the group consisting of methyl, ethyl, and isopropyl, which is
optionally substituted with 1-3 R⁴;
the C₁-C₆ alkyl-hydroxy is hydroxymethyl; and
the C₁-C₆ alkyl-amino is —CH₂—CH₂—NH—;
in R⁴:
the R^{4a} in S(═O)₂R^{4a} is selected from the group consisting of methyl and phenyl; and
the R^{4a} in C(═O)R^{4a} is methyl;
in R⁵:
the C₁-C₆ alkyl is methyl;
in R⁶:
the C₁-C₆ alkyl is methyl; and
in R⁸:
the C₁-C₆ alkyl is methyl; and
the C₁-C₆ alkoxy is methoxy.

In some embodiments, for the compound of Formula (TT) or the pharmaceutically acceptable salt or solvate thereof, [symbol] is a single bond.

In some embodiments, for the compound of Formula (II) or the pharmaceutically acceptable salt or solvate thereof, [symbol] is a double bond.

In some embodiments, for the compound of Formula (II) or the pharmaceutically acceptable salt or solvate thereof, [symbol] is a single bond, and A is CR².

In some embodiments, for the compound of Formula (II) or the pharmaceutically acceptable salt or solvate thereof, [symbol] is a single bond, A is CR², and n is 1.

In some embodiments, for the compound of Formula (II) or the pharmaceutically acceptable salt or solvate thereof, [symbol] is a single bond, A is CR², and n is 0.

In some embodiments, for the compound of Formula (TT) or the pharmaceutically acceptable salt or solvate thereof, [symbol] is a single bond, A is CR², and n is 2.

In some embodiments, for the compound of Formula (II) or the pharmaceutically acceptable salt or solvate thereof, [symbol] is a single bond, A is NR¹.

In some embodiments, for the compound of Formula (II) or the pharmaceutically acceptable salt or solvate thereof, [symbol] is a single bond, A is NR¹, and n is 1.

In some embodiments, for the compound of Formula (II) or the pharmaceutically acceptable salt or solvate thereof, [symbol] is a single bond, and A is O.

In some embodiments, for the compound of Formula (II) or the pharmaceutically acceptable salt or solvate thereof, [symbol] is a single bond, A is CR², and n is 1, the compound is selected from the group consisting of

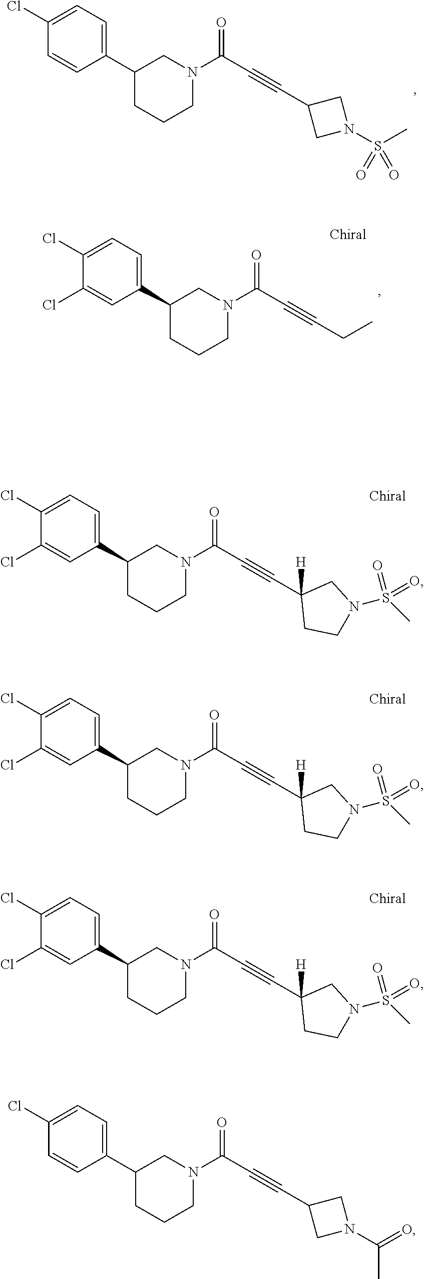 [SMILES: CS(=O)(=O)N1CC(C#CC(=O)N2CCCC(c3ccc(Cl)cc3)C2)C1 ; CCC#CC(=O)N1CCC[C@@H](c2ccc(Cl)c(Cl)c2)C1 ; CS(=O)(=O)N1CC[C@H](C#CC(=O)N2CCC[C@@H](c3ccc(Cl)c(Cl)c3)C2)C1 ; CS(=O)(=O)N1CC[C@H](C#CC(=O)N2CCC[C@@H](c3ccc(Cl)c(Cl)c3)C2)C1 ; CS(=O)(=O)N1CC[C@H](C#CC(=O)N2CCC[C@@H](c3ccc(Cl)c(Cl)c3)C2)C1 ; CC(=O)N1CC(C#CC(=O)N2CCCC(c3ccc(Cl)cc3)C2)C1]

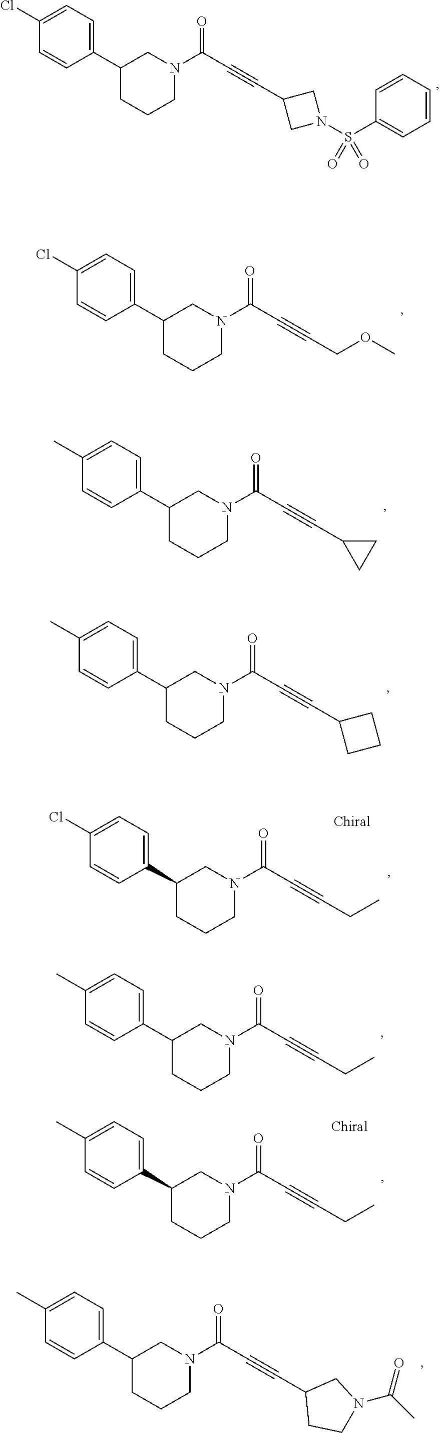

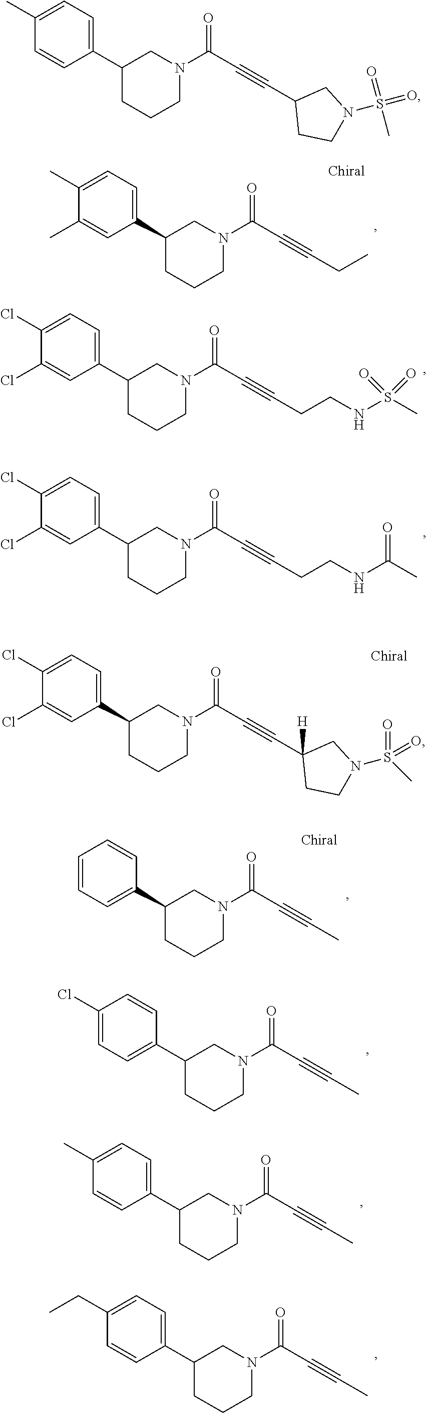

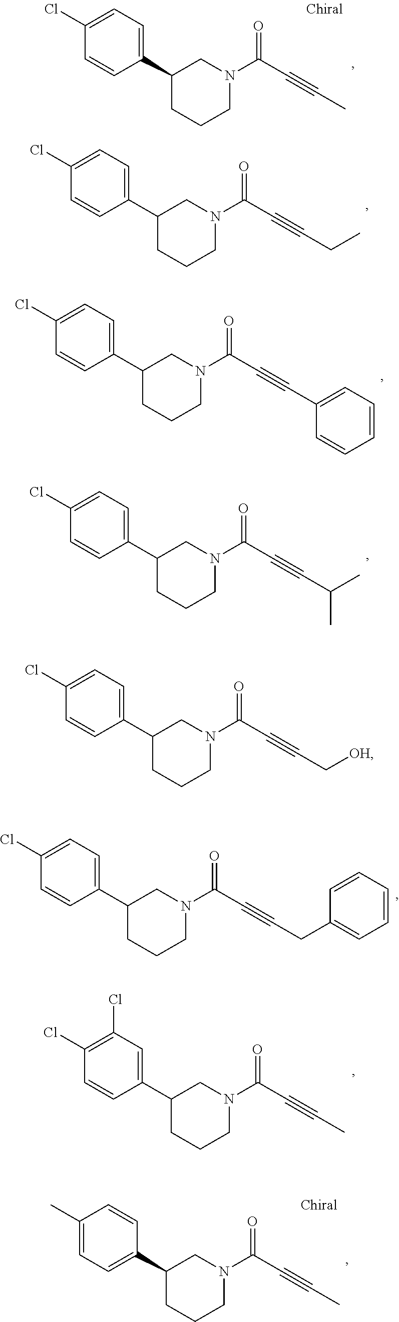

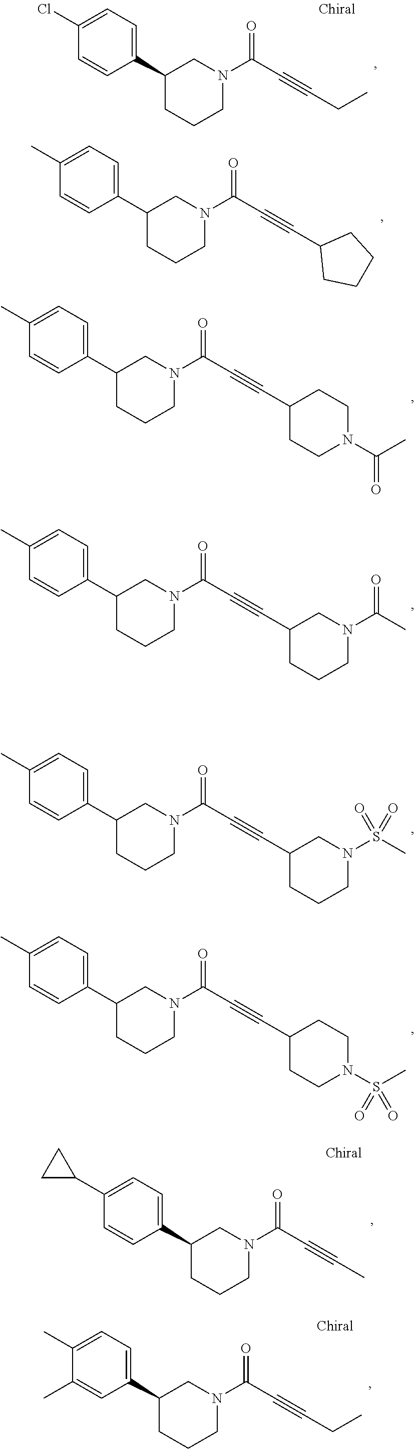

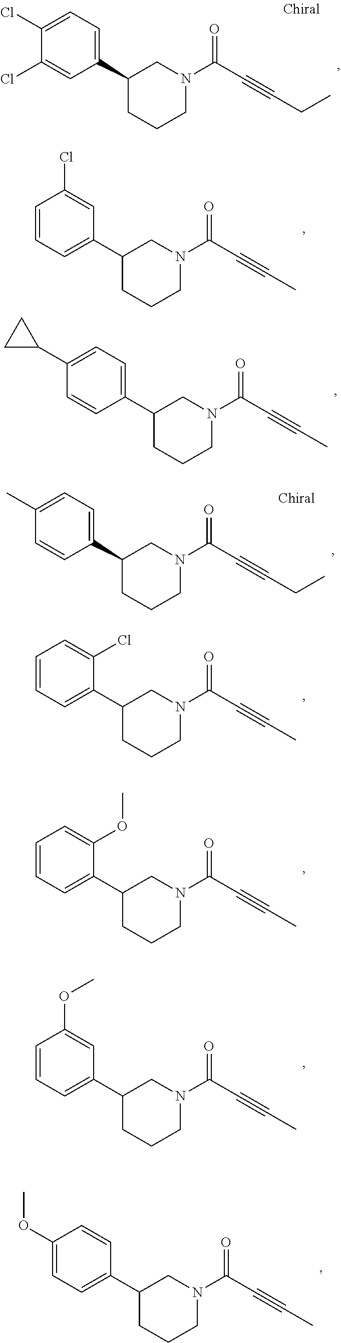

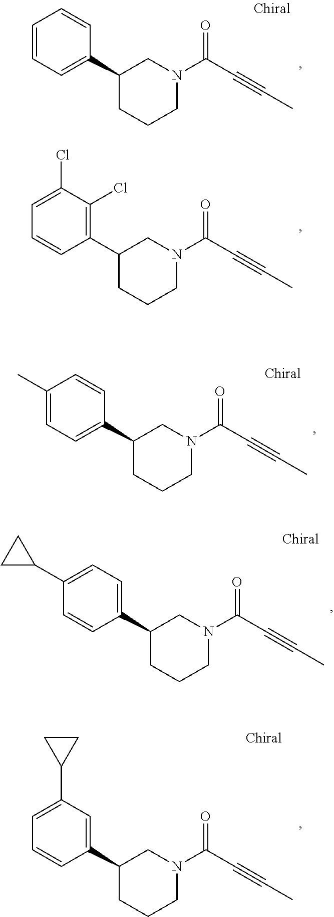

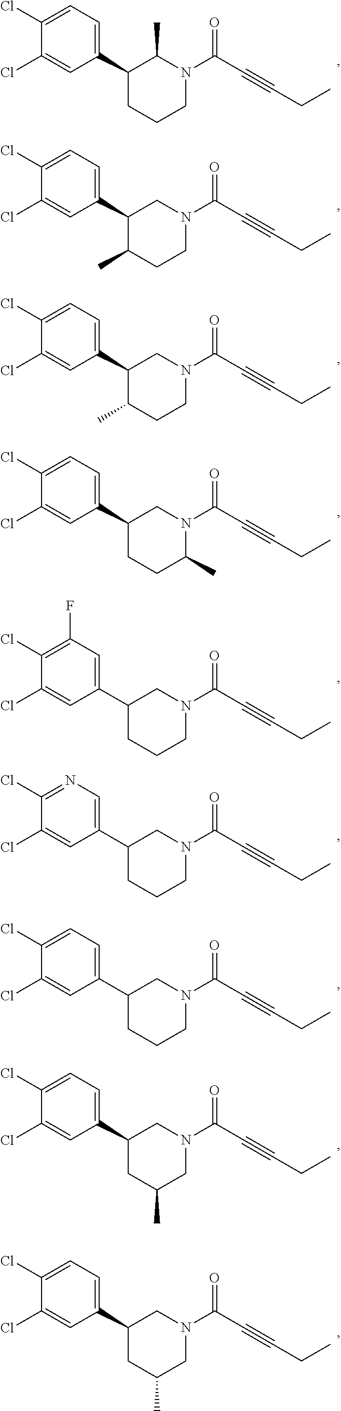

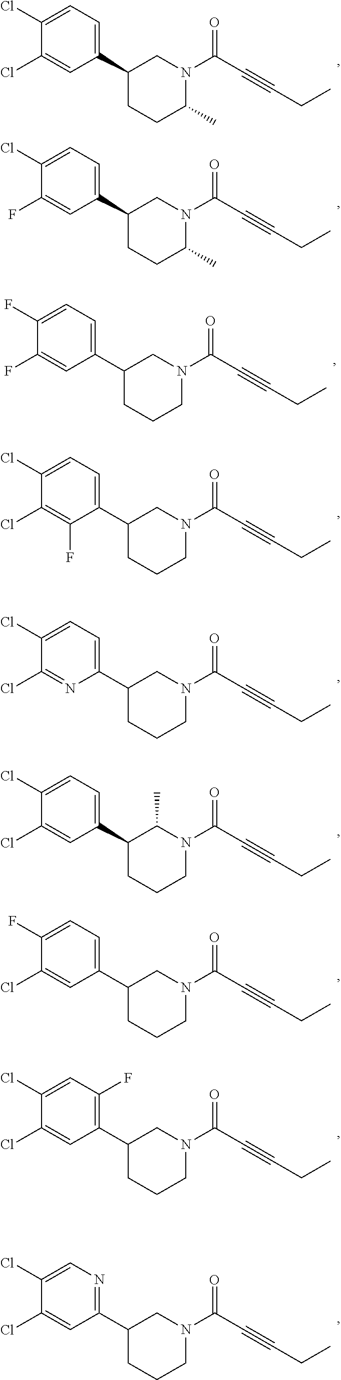

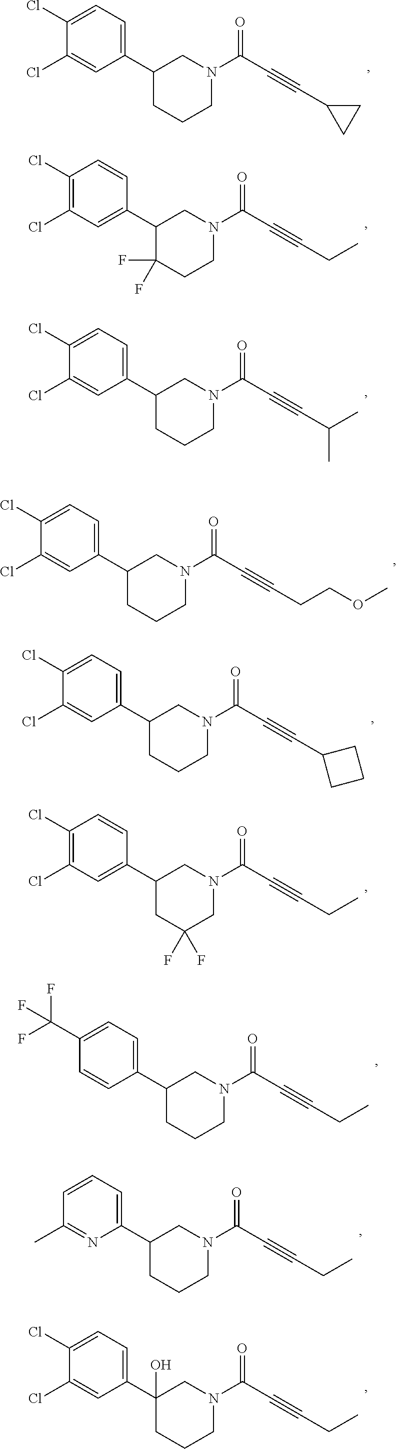

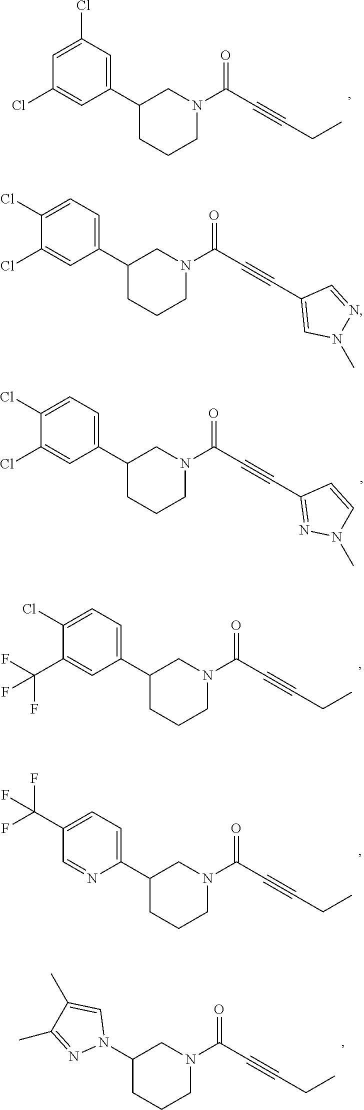

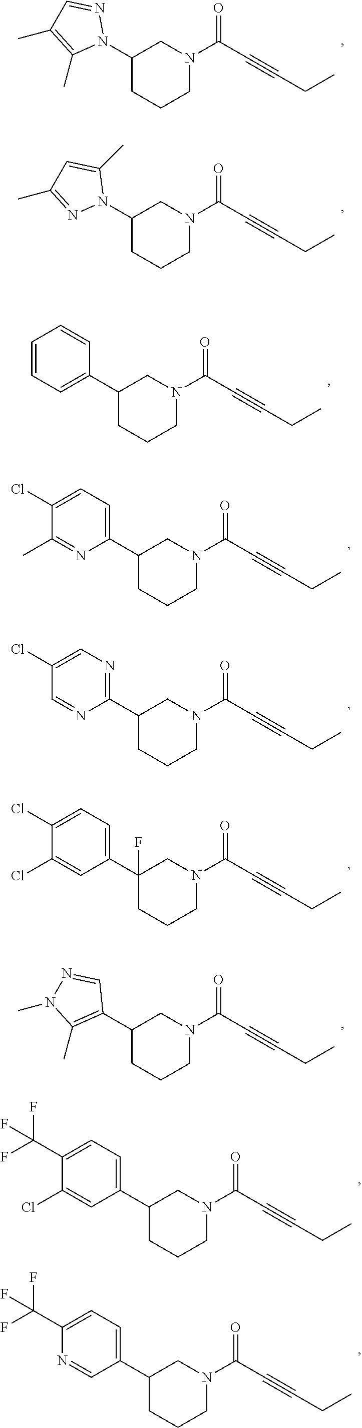

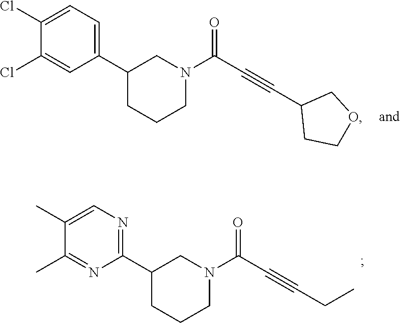 [SMILES: O=C(C#CC1CCOC1)N1CCCC(c2ccc(Cl)c(Cl)c2)C1 ; CCC#CC(=O)N1CCCC(c2ncc(C)c(C)n2)C1]

or a pharmaceutically acceptable salt or solvate thereof.

In some embodiments, for the compound of Formula (II) or the pharmaceutically acceptable salt or solvate thereof, [symbol] is a double bond, A is CR², and n is 0, the compound is selected from the group consisting of:

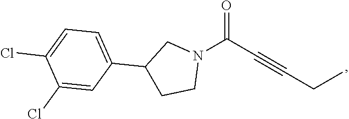 [SMILES: CCC#CC(=O)N1CCC(c2ccc(Cl)c(Cl)c2)C1]

or a pharmaceutically acceptable salt or solvate thereof.

In some embodiments, for the compound of Formula (II) or the pharmaceutically acceptable salt or solvate thereof, [symbol] is a double bond, A is CR², and n is 2, the compound is selected from the group consisting of:

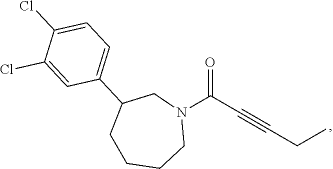 [SMILES: CCC#CC(=O)N1CCCCC(c2ccc(Cl)c(Cl)c2)C1]

or a pharmaceutically acceptable salt or solvate thereof.

In some embodiments, for the compound of Formula (II) or the pharmaceutically acceptable salt or solvate thereof, wherein [symbol] is a single bond, A is NR¹, and n is 1, the compound is selected from the group consisting of:

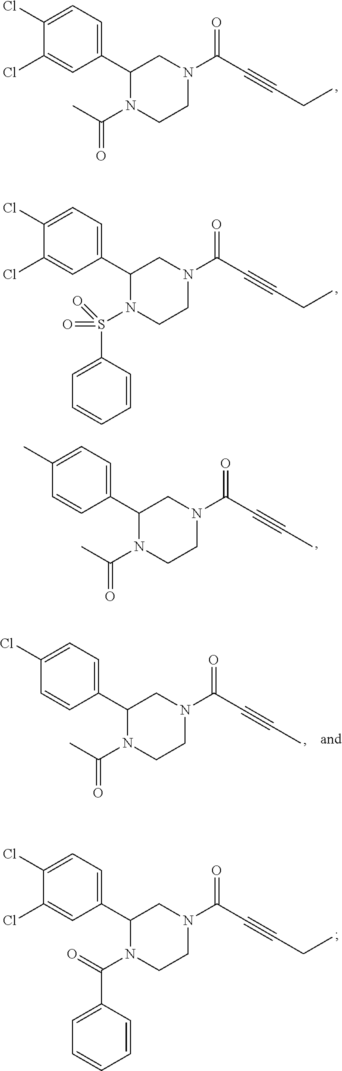

or a pharmaceutically acceptable salt or solvate thereof.

In some embodiments, for the compound of Formula (II) or the pharmaceutically acceptable salt or solvate thereof, wherein [symbol] is a single bond, and A is O, the compound is selected from the group consisting of:

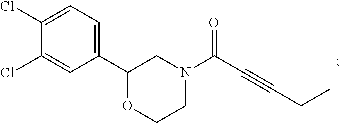 [SMILES: CCC#CC(=O)N1CCOC(c2ccc(Cl)c(Cl)c2)C1]

or a pharmaceutically acceptable salt or solvate thereof.

In some embodiments, for the compound of Formula (II) or the pharmaceutically acceptable salt or solvate thereof, wherein [symbol] is a double bond, A is CR, and n is 1, the compound is selected from the group consisting of:

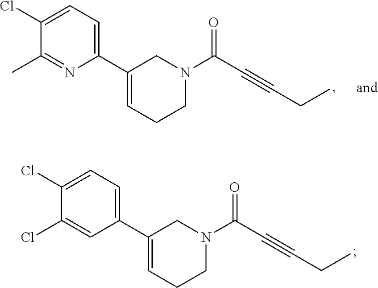 [SMILES: CCC#CC(=O)N1CCC=C(c2ccc(Cl)c(C)n2)C1 ; CCC#CC(=O)N1CCC=C(c2ccc(Cl)c(Cl)c2)C1]

or a pharmaceutically acceptable salt or solvate thereof.

In one aspect disclosed herein is a compound of Formula (II), or a pharmaceutically acceptable salt or solvate thereof:

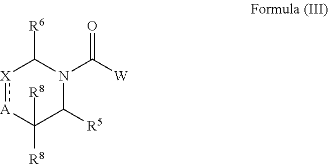

wherein:
   [symbol] is a single or double bond;
   X is C(J)(R⁷) when [symbol] is a single bond and C(J) when [symbol] is a double bond;
A is CR², NR¹, O or S or S(═O)₂ when [symbol] is a single bond; and CR when [symbol] is a double bond;
each R independently is H, halo, or C₁-C₆ alkyl;
R¹ is selected from the group C₁-C₆ alkyl, S(═O)₂—(C₁-C₆)alkyl, S(═O)₂—(C₆-C₁₀) aryl, C(═O)—(C₁-C₆) alkyl, or C(═O)—(C₆-C₁₀)aryl;
J is a C₆-C₁₀ aryl or a five- to six-membered heteroaryl, each of which is optionally substituted with 1-4 R³;
each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halo, C₁-C₆ haloalkyl, cyano and N(R⁴)₂; or two R³ optionally are taken together with the carbon atoms to which they are attached to form a five- or six-membered heterocycle or a C₃-C₆ cycloalkyl;
R is selected from the group consisting of hydrogen, C₁-C₆ alkyl, hydroxy, C₁-C₆ alkoxy, halogen, and —N(R³)₂;
R⁶ is H or C₁-C₆ alkyl;
R⁷ is H, hydroxy, or C₁-C₆ alkyl;
each R independently is H, halo, C₁-C₆ alkoxy, or C₁-C₆ alkyl;
n is 0, 1, or 2; and
W comprises an electrophile that reacts and forms a covalent bond with the sulfur atom at cysteine 817 as set forth in SEQ ID NO: 1, 2, 3, or 4.

In some embodiments, for the compound of Formula (III) or the pharmaceutically acceptable salt or solvate thereof, W comprises an alkynyl moiety.

In some embodiments, for the compound of Formula (III) or the pharmaceutically acceptable salt or solvate thereof, W comprises a moiety of chemical structure:

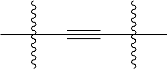 [SMILES: *C#C*]

wherein:
[symbol] represents points of attachment of the moiety to the remainder of W.

In some embodiments, for the compound of Formula (III):
in J:
   the optionally substituted C₆-C₁₀ aryl is phenyl which is optionally substituted with 1-4 R³;
   the optionally substituted five- to six-membered heteroaryl is selected from the group consisting of pyridyl, pyrazolyl, and pyrimidinyl, each of which is optionally substituted with 1-4 R³;
   in R¹:
   the C₁-C₆ alkyl is methyl;
   the C₁-C₆ alkyl in S(═O)₂—(C₁-C₆)alkyl is methyl;
   the C₆-C₁₀ aryl in S(═O)₂—(C₆-C₁₀) aryl is phenyl;
   the C₁-C₆ alkyl in C(═O)—(C₁-C₆) alkyl is methyl; and
   the C₆-C₁₀ aryl in C(═O)—(C₆-C₁₀) aryl is phenyl;
   in R³:
   the C₁-C₆ alkyl is selected from the group consisting of methyl, and ethyl;
   the C₃-C₆ cycloalkyl is cyclopropyl;
   the C₁-C₆ alkoxy is methoxy; and
   the C₁-C₆ haloalkyl is trifluoromethyl;
   in R²:
   the four- to six-membered heterocyclyl is selected from the group consisting of azetidinyl,
   tetrahydrofuranyl, piperidinyl, and pyrrolidinyl, each of which is optionally substituted with 1-3 R⁴;
   the C₆-C₁₀ aryl is phenyl which is optionally substituted with 1-3 R⁴;
   the C₃-C₈ cycloalkyl is selected from the group consisting of cyclopropyl, cyclobutyl, and
   cyclopentyl, each of which is optionally substituted with 1-3 R⁴;
   the five- to six-membered heteroaryl is pyrazolyl which is optionally substituted with 1-3 R⁴;
   the C₁-C₆ alkyl is selected from the group consisting of methyl, ethyl, and isopropyl, which is optionally substituted with 1-3 R⁴;
   the C₁-C₆ alkyl-hydroxy is hydroxymethyl; and
   the C₁-C₆ alkyl-amino is —CH₂—CH₂—NH—;
   in R⁴:
   the R^{4a} in S(═O)₂R^{4a} is selected from the group consisting of methyl and phenyl; and
   the R^{4a} in C(═O)R^{4a} is methyl;
   in R⁵:
   the C₁-C₆ alkyl is methyl;
   in R⁶:
   the C₁-C₆ alkyl is methyl; and
   in R⁸:
   the C₁-C₆ alkyl is methyl; and
   the C₁-C₆ alkoxy is methoxy.

[symbol]

In some embodiments, In some embodiments, in Formula (III), [symbol] is a single bond.

In some embodiments, in Formula (III), [symbol] is a double bond.

In some embodiments, in Formula (III), [symbol] is a single bond, and A is CR².

In some embodiments, in Formula (III), [symbol] is a single bond, A is CR², and n is 1.

In some embodiments, in Formula (III), [symbol] is a single bond, A is CR², and n is 0.

In some embodiments, in Formula (III), [symbol] is a single bond, A is CR², and n is 2.

In some embodiments, in Formula (III), [symbol] is a single bond, and A is NR¹.

In some embodiments, in Formula (III), [symbol] is a single bond, A is NR¹, and n is 1.

In some embodiments, in Formula (III), [symbol] is a single bond, and A is O.

In some embodiments, in Formula (III), [symbol] is a double bond, and n is 1.

In some embodiments, wherein in Formula (III), [symbol] is a single bond, A is CR², and n is 1,

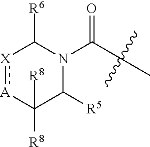

is selected from the group consisting of

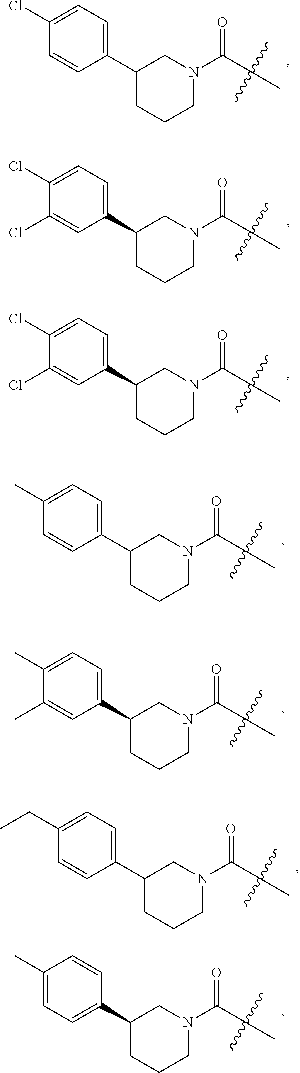

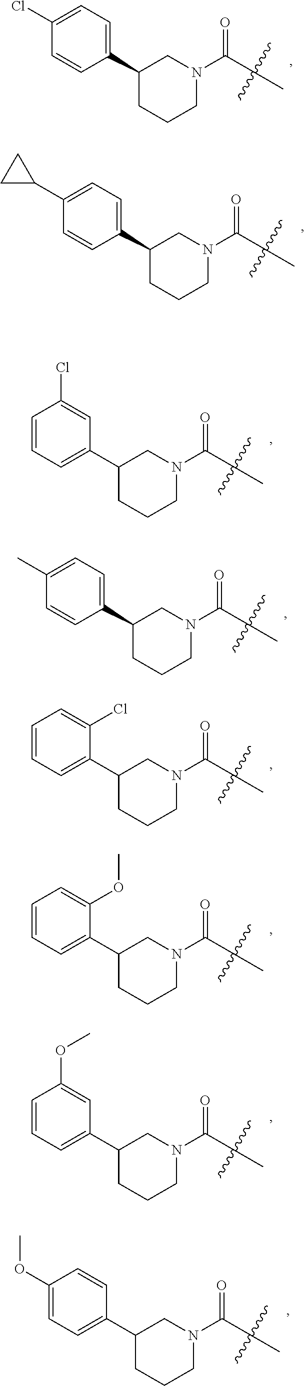

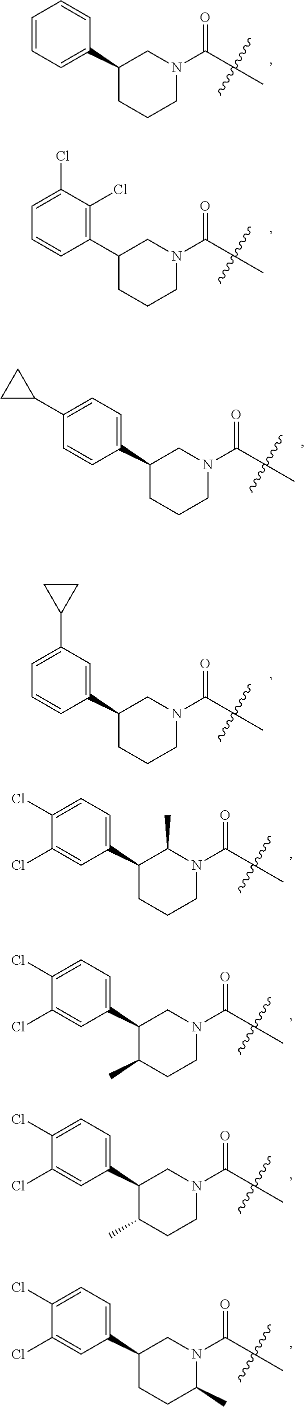

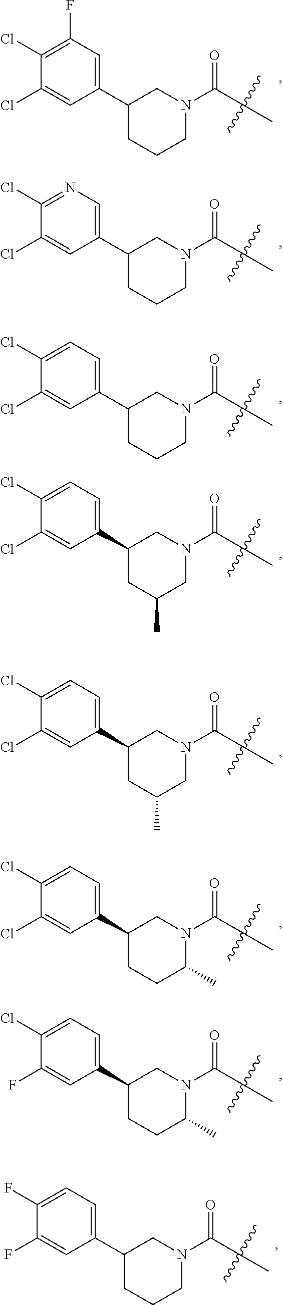

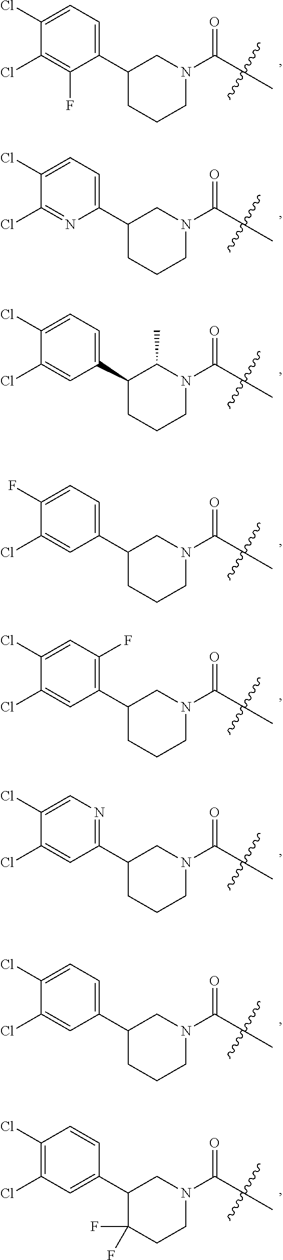

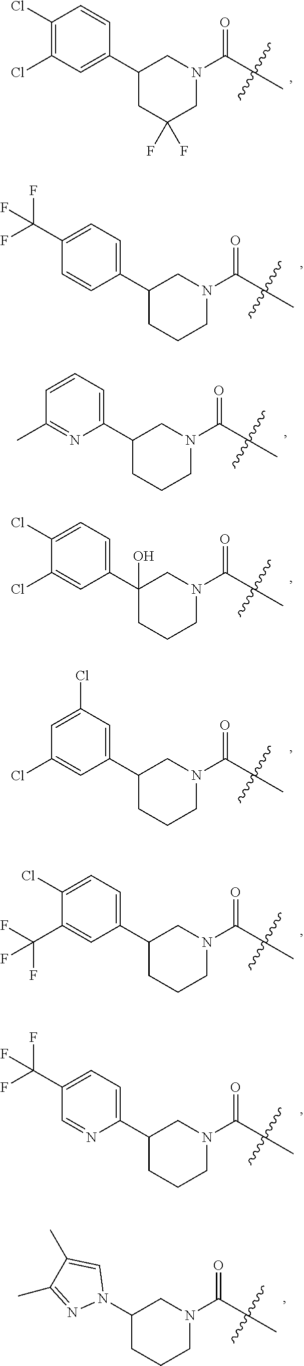

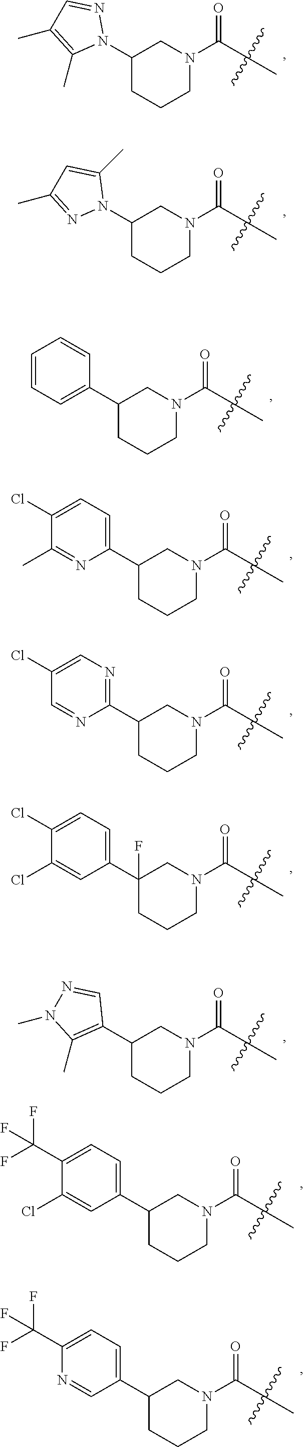

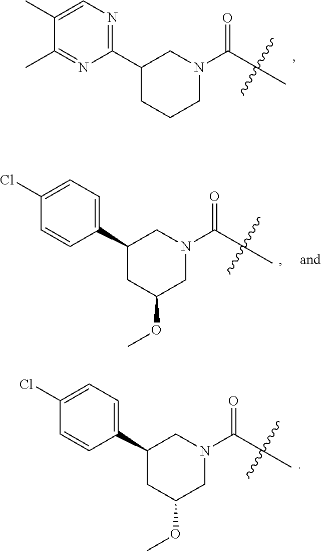 [SMILES: *C(=O)N1CCCC(c2ncc(C)c(C)n2)C1 ; *C(=O)N1C[C@@H](OC)C[C@@H](c2ccc(Cl)cc2)C1 ; *C(=O)N1C[C@H](OC)C[C@@H](c2ccc(Cl)cc2)C1]

In some embodiments, wherein in Formula (III), [symbol] is a single bond, A is CR², and n is 0,

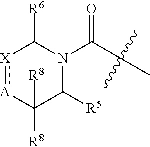

is selected from the group consisting of

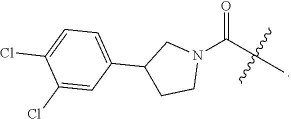 [SMILES: *C(=O)N1CCC(c2ccc(Cl)c(Cl)c2)C1]

In some embodiments, wherein in Formula (III), [symbol] is a single bond, A is CR², and n is 2,

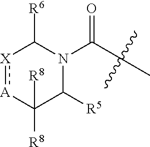

is selected from the group consisting of:

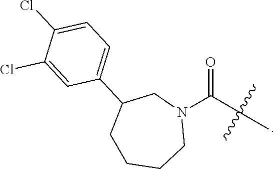 [SMILES: *C(=O)N1CCCCC(c2ccc(Cl)c(Cl)c2)C1]

In some embodiments, wherein in Formula (III), [symbol] is a single bond, A is NR¹, and n is 1,

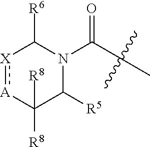

is selected from the group consisting of:

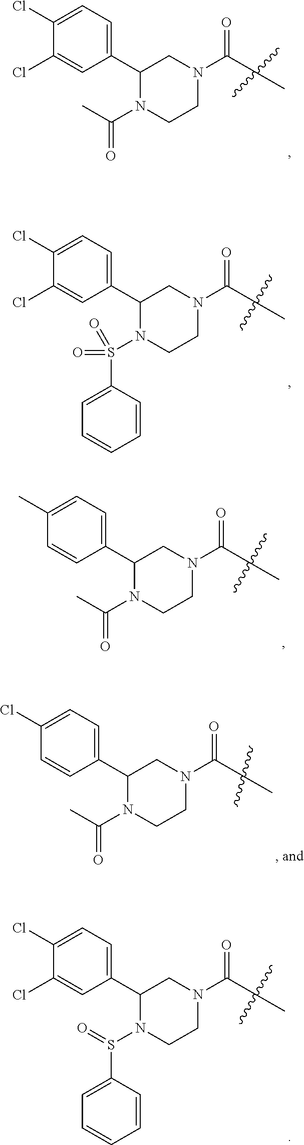 [SMILES: *C(=O)N1CCN(C(C)=O)C(c2ccc(Cl)c(Cl)c2)C1 ; *C(=O)N1CCN(S(=O)(=O)c2ccccc2)C(c2ccc(Cl)c(Cl)c2)C1 ; *C(=O)N1CCN(C(C)=O)C(c2ccc(C)cc2)C1 ; *C(=O)N1CCN(C(C)=O)C(c2ccc(Cl)cc2)C1 ; *C(=O)N1CCN(S(=O)c2ccccc2)C(c2ccc(Cl)c(Cl)c2)C1]

In some embodiments, in Formula (III), wherein [symbol] is a single bond, and A is O,

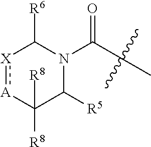

is selected from the group consisting of:

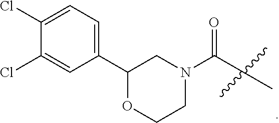 [SMILES: *C(=O)N1CCOC(c2ccc(Cl)c(Cl)c2)C1]

In some embodiments, wherein in Formula (III), [symbol] is a double bond, and n is 1,

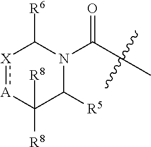

is selected from the group consisting of:

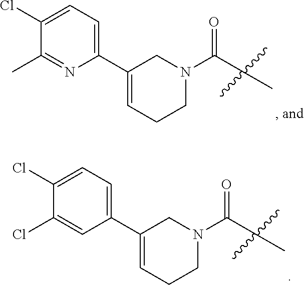 [SMILES: *C(=O)N1CCC=C(c2ccc(Cl)c(C)n2)C1 ; *C(=O)N1CCC=C(c2ccc(Cl)c(Cl)c2)C1]

In another aspect disclosed herein is a compound of Formula (I), or a pharmaceutically acceptable salt or solvate thereof:

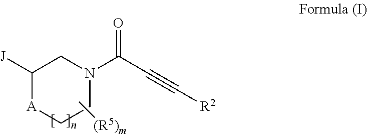

wherein:
   A is C(R⁵)₂ or NR¹;
   R¹ is selected from the group CH₃, SO₂CH₃, SO₂C₆-aryl, C(O)CH₃, or C(O)C₆-aryl;
   J is a C₆-C₁₂ aryl optionally substituted with 1, 2, 3, 4, 5, 6 or 7 R³;
   each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halogen, C₁-C₆ haloalkyl, cyano and N(R⁴)₂; or two R³ optionally join together to form a heterocycle having 3-12 ring atoms or a C₃-C₆ cycloalkyl;
   R² is selected from the group consisting of C₆-C₁₂ aryl, C₅-C₁₂ heteroaryl, C₁-C₆ alkyl, C₃-C₆ cycloalkyl, C₁-C₆ alkyl-hydroxy, C₁-C₆ alkoxy, C₁-C₆ alkyl-amino, C₂-C₆ alkenyl, C₂-C₆ alkynyl, C₁-C₆ haloalkyl, or 3-12 membered heterocycle, each of which is optionally substituted with one or more R⁴;
   each R⁴ is independently selected from the group consisting of hydrogen, hydroxyl, C₁-C₆ alkoxy and C₁-C₆ alkyl, C(O)R^{4a} or SO₂R^{4a}; or two R⁴ optionally join together to form a heterocycle having 3-12 ring atoms or C₃-C₆ cycloalkyl, wherein R^{4a} is hydrogen or C₁-C₆ alkyl;
   each R⁵ is independently selected from the group consisting of hydrogen, C₁-C₆ alkyl, hydroxy, C₁-C₆ alkoxy, halogen, and —N(R³)₂;
   n is an integer selected from 0, 1, or 2; and
   m is an integer selected from 0, 1, 2, 3, 4, or 5.

In another aspect disclosed herein is a compound of Formula (I), or a pharmaceutically acceptable salt or solvate thereof:

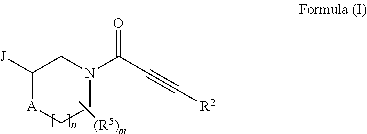

wherein:
   A is CH₂ or NR¹;
   R¹ is selected from the group CH₃, SO₂CH₃, SO₂C₆-aryl, C(O)CH₃, or C(O)C₆-aryl;
   J is a C₆-C₁₂ aryl optionally substituted with 1, 2, 3, 4, 5, 6 or 7 R³;
   each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halogen, C₁-C₆ haloalkyl, cyano and N(R⁴)₂; or two R³ optionally joined together to form a heterocycle having 3-12 ring atoms or a C₃-C₆ cycloalkyl;
   R² is selected from the group consisting of C₆-C₁₂ aryl, C₅-C₁₂ heteroaryl, C₁-C₆ alkyl, C₃-C₆ cycloalkyl, C₁-C₆ alkyl-hydroxy, C₁-C₆ alkoxy, C₁-C₆ alkyl-amino, C₂-C₆ alkenyl, C₂-C₆ alkynyl, C₁-C₆ haloalkyl, or 3-12 membered heterocycloalkyl, each of which is optionally substituted with one or more R⁴;
   each R⁴ is independently selected from the group consisting of hydrogen, hydroxyl, C₁-C₆ alkoxy and C₁-C₆ alkyl, C(O)R^{4a} or SO₂R^{4a} or two R⁴ optionally join to form a heterocycle having 3-12 ring atoms or C₃-C₆ cycloalkyl, wherein R^{4a} is hydrogen or C₁-C₆ alkyl;
   each R⁵ is independently selected from the group consisting of hydrogen, C₁-C₆ alkyl, hydroxy, C₁-C₆ alkoxy, halogen, and —N(R³)₂;
   n is 1; and
   m is 1, 2, 3, or 4.

In another aspect disclosed herein is a compound of Formula (I), or a pharmaceutically acceptable salt or solvate thereof.

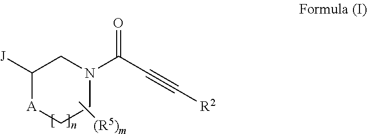

wherein:
   A is CH₂ or NR¹;
   R¹ is selected from the group CH₃, SO₂CH₃, SO₂C₆-aryl, C(O)CH₃, or C(O)C₆-aryl;
   J is a C₆ aryl optionally substituted with 1, 2, 3, or 4 R³;
   each R³ is independently selected from the group consisting of C₁-C₃ alkyl, C₃-C₄ cycloalkyl, C₁-C₃ alkyl-hydroxy, C₁-C₃ alkyl-amino, C₁-C₃ alkoxy, hydroxyl, C₂-C₄ alkenyl, C₂-C₄ alkynyl, halogen, cyano, and N(R⁴)₂ or two R³ optionally joined to form a heterocycle having 3-12 ring atoms or a C₃-C₄ cycloalkyl;
   R² is selected from the group consisting of C₆-C₁₂ aryl, C₅-C₁₂ heteroaryl, C₁-C₄ alkyl, C₃-C₆ cycloalkyl, C₁-C₃ alkyl-hydroxy, C₁-C₃ alkyl-amino, pyrrolidine-SO₂CH₃, or 3-7 membered heterocycloalkyl, wherein each is optionally substituted with one or more R⁴;
   each R⁴ is independently selected from the group consisting of hydrogen, hydroxyl, C₁-C₆ alkoxy and C₁-C₆ alkyl, or two R⁴ optionally join together to form heterocycle having 3-12 ring atoms or C₃-C₆ cycloalkyl;
   each R⁵ is independently selected from the group consisting of hydrogen, C₁-C₂ alkyl, hydroxy, C₁-C₃ alkoxy and —N(R³)₂;
   n is 1; and
   m is 0, 1, 2, or 3.

In another aspect disclosed herein is a compound of Formula (I), or a pharmaceutically acceptable salt or solvate:

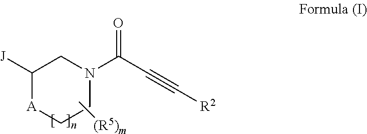

wherein:
   A is CH₂ or NR¹;
   R¹ is selected from the group CH₃, SO₂CH₃, SO₂C₆-aryl, C(O)CH₃, or C(O)C₆-aryl;
   J is a C₆ aryl optionally substituted with 1 or 2 R³;
   each R³ is independently selected from the group consisting of C₁-C₂ alkyl, C₃ cycloalkyl, C₁ alkoxy, or chloride;
   R² is selected from the group consisting of C₆ aryl, C₁-C₂ alkyl, C₃-C₈ cycloalkyl, C₁-C₃ alkyl-hydroxy, C₁-C₄ alkyl-amino, or 4-6 membered heterocycloalkyl ring, each or which is optionally substituted with one or more R⁴;
   each R⁴ is independently selected from the group consisting of hydrogen, C₁ alkoxy, C₁ alkyl, C(O)CH₃ and SO₂CH₃;
   each R⁵ is hydrogen; and
   n is 1.

In some embodiments, J is an optionally substituted C₆ aryl. In some embodiments, J is phenyl optionally substituted with 1, 2, 3, or 4 R³. In some embodiments, J is phenyl optionally substituted with 1, 2, or 3 R³. In some embodiments, J is phenyl optionally substituted with 1 or 2 R³. In some embodiments, J is phenyl optionally substituted with 1 R³. In some embodiments, J is unsubstituted phenyl.

In some embodiments, n is 1. In some embodiments, n is 2. In some embodiments, n is 0.

In some embodiments, the compound of Formula (I) has the structure of Formula (II), or a pharmaceutically acceptable salt or solvate thereof:

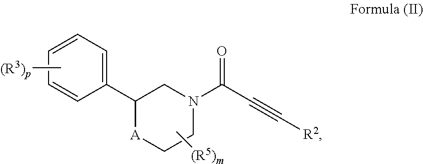

wherein p is an integer selected from 0, 1, 2, 3, 4, or 5; and the other variables are described herein.

In some embodiments, A is C(R)₂, In some embodiments, A is CH₂.

In some embodiments, A is NR¹.

In some embodiments, the compound of Formula (I) has the structure of Formula (IIa), or a pharmaceutically acceptable salt or solvate thereof:

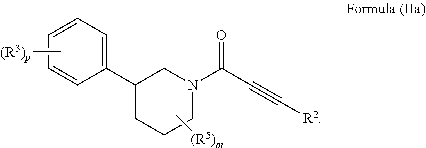

In some embodiments, the compound of Formula (I) has the structure of Formula (IIb), or a pharmaceutically acceptable salt or solvate thereof:

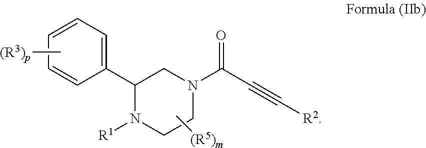

In some embodiments, p is 0, 1, 2, 3, or 4. In some embodiments, p is 1 or 2. In some embodiments, p is 1. In some embodiments, p is 2. In some embodiments, p is 0.

In some embodiments, R¹ is selected from the group C₁-C₃ alkyl, C(O)—C₁-C₃ alkyl, and C(O)C₆-cycloalkyl. In some embodiments, R¹ is selected from the group CH₃, SO₂CH₃, SO₂C₆-aryl, C(O)CH₃, or C(O)C₆-aryl. In some embodiments, R¹ is CH₃. In some embodiments, R¹ is C(O)CH₃. In some embodiments, R₁ is C(O)-cyclohexyl.

In some embodiments, R² is selected from the group consisting of C₆-C₁₂ aryl, C₅-C₁₂ heteroaryl, C₁-C₄ alkyl, C₃-C₆ cycloalkyl, C₁-C₃ alkyl-hydroxy, C₁-C₃ alkyl-amino, pyrrolidine-SO₂CH₃. In some embodiments, R² is selected from the group consisting of C₆-C₁₂ aryl, C₅-C₁₂ heteroaryl, C₁-C₆ alkyl, C₃-C₆ cycloalkyl, C₁-C₆ alkyl-hydroxy, C₁-C₆ alkoxy, C₁-C₆ alkyl-amino, C₂-C₆ alkenyl, C₂-C₆ alkynyl, C₁-C₆ haloalkyl, or 3-12 membered heterocycle. In some embodiments, R² is selected from the group consisting of C₆-C₁₂ aryl, C₅-C₁₂ heteroaryl, C₁-C₄ alkyl, C₃-C₆ cycloalkyl, C₁-C₃ alkyl-hydroxy, C₁-C₃ alkyl-amino, pyrrolidine-SO₂CH₃, or 3-7 membered heterocycloalkyl.

In some embodiments, each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halogen, C₁-C₆ haloalkyl, cyano and N(R⁴)₂. In some embodiments, each R³ is independently selected from the group consisting of C₁-C₂ alkyl, C₃ cycloalkyl, C₁ alkoxy, or chloride. In some embodiments, each R³ is independently C₁-C₂ alkyl. In some embodiments, each R³ is independently methyl or ethyl. In some embodiments, each R³ is independently cyclopropyl. In some embodiments, each R³ is independently —OH. In some embodiments, each R³ is independently halogen. In some embodiments, each R³ is independently fluorine, bromine, or chlorine. In some embodiments, each R³ is independently chlorine.

In some embodiments, two R³ optionally joined to form a heterocycloalkyl having 3-12 ring atoms or a C₃-C₆ cycloalkyl optionally substituted with one or more R⁴. In some embodiments, two R³ join together to form a heterocycloalkyl having 3-12 ring atoms. In some embodiments, two R³ join together to form a C₃-C₆ cycloalkyl.

In some embodiments, each R⁴ is independently selected from the group consisting of hydrogen, hydroxyl, C₁-C₆ alkoxy and C₁-C₆ alkyl, C(O)R^{4a} or SO₂R^{4a}, wherein R^{4a} is hydrogen or C₁-C₆ alkyl. In some embodiments, each R⁴ is independently selected from the group consisting of —OH, OCH₃, C(O)CH₃, or SO₂CH₃.

In some embodiments, or two R⁴ optionally join together to form a heterocycle having 3-12 ring atoms or C₃-C₆ cycloalkyl. In some embodiments, two R⁴ join together to form a heterocycloalkyl having 3-12 ring atoms. In some embodiments, two R⁴ join together to form a C₃-C₆ cycloalkyl.

In some embodiments, each R⁴ is independently selected from the group consisting of hydrogen, C₁-C₂ alkyl, hydroxy, C₁-C₃ alkoxy and —N(R³)₂. In some embodiments, R⁵ is hydrogen.

In some embodiments, n is 0, 1, 2, 3, 4, or 5. In some embodiments, in is 0, 1, 2 or 3. In some embodiments, m is 1. In some embodiments, m is 2. In some embodiments, m is 3. In some embodiments, m is 0.

In another aspect, provided herein is a pharmaceutical composition comprising a compound described herein (including corresponding to a compound according to Formula (I), (II), or (III)), or a pharmaceutically acceptable salt or solvate thereof, and a pharmaceutically acceptable excipient.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Modified JAK1 Protein-Small Molecule Conjugate

In one aspect, provided herein is a modified JAK1 protein comprising a non-naturally occurring small molecule fragment having a covalent bond to cysteine 817 of the JAK1 protein, wherein the modified JAK1 protein comprises SEQ ID NO: 1, 2, 3, or 4; and has the structure of Formula (IV):

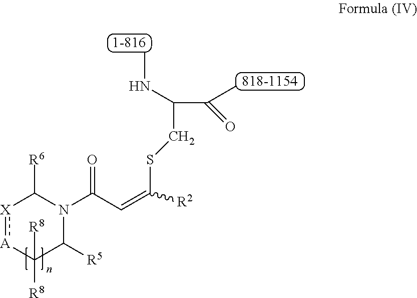

wherein:
[symbol] is a single or double bond;
X is C(J)(R⁷) when [symbol] is a single bond and C(J) when [symbol] is a double bond;
A is CR², NR¹, O or S or S(═O)₂ when [symbol] is a single bond; and CR when [symbol] is a double bond;
   each R independently is H, halo, or C₁-C₆ alkyl;
R¹ is selected from the group C₁-C₆ alkyl, S(═O)₂—(C₁-C₆)alkyl, S(═O)₂—(C₆-C₁₀) aryl, C(═O)—(C₁-C₆) alkyl, or C(═O)—(C₆-C₁₀)aryl;
J is a C₆-C₁₀ aryl or a five- to six-membered heteroaryl, each of which is optionally substituted with 1-4 R³
each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halo, C₁-C₆ haloalkyl, cyano and N(R⁴)₂; or two R³ optionally are taken together with the carbon atoms to which they are attached to form a five- or six-membered heterocycle or a C₃-C₆ cycloalkyl;
R² is selected from the group consisting of C₆-C₁₀ aryl, five- or six-membered heteroaryl, C₁-C₆ alkyl, C₃-C₈ cycloalkyl, C₁-C₆ alkyl-hydroxy, C₁-C₆ alkoxy, C₁-C₆ alkyl-amino, C₂-C₆ alkenyl, C₂-C₆ alkynyl, C₁-C₆ haloalkyl, or four- to six-membered heterocyclyl, each of which is optionally substituted with 1-3 R⁴;
each R⁴ is independently selected from the group consisting of hydroxyl, C₁-C₆ alkoxy, C₁-C₆ alkyl, C(═O)R^{4a} or S(═O)₂R^{4a} or wherein two R⁴ optionally are taken together with the carbon atoms to which they are attached to form a five- to six-membered heterocyclyl or a C₃-C₆ cycloalkyl, wherein each R^{4a} independently is hydrogen or C₁-C₆ alkyl;
R⁵ is selected from the group consisting of hydrogen, C₁-C₆ alkyl, hydroxy, C₁-C₆ alkoxy, halogen, and —N(R³)₂;
R⁶ is H or C₁-C₆ alkyl;
R⁷ is H, hydroxy, or C₁-C₆ alkyl;
each R⁸ independently is H, halo, C₁-C₆ alkoxy, or C₁-C₆ alkyl; and
n is 0, 1, or 2; and

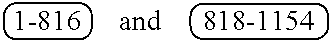

and represent amino acid sequences 1-816 and 818-1154 respectively in SEQ ID NO: 1, 2, 3, or 4.

In some embodiments, in Formula (IV):
in J:
the optionally substituted C₆-C₁₀ aryl is phenyl which is optionally substituted with 1-4 R³;
the optionally substituted five- to six-membered heteroaryl is selected from the group consisting of pyridyl, pyrazolyl, and pyrimidinyl, each of which is optionally substituted with 1-4 R³;
in R¹:
the C₁-C₆ alkyl is methyl;
the C₁-C₆ alkyl in S(═O)₂—(C₁-C₆)alkyl is methyl;
the C₆-C₁₀ aryl in S(═O)₂—(C₆-C₁₀) aryl is phenyl;
the C₁-C₆ alkyl in C(═O)—(C₁-C₆) alkyl is methyl; and
the C₆-C₁₀ aryl in C(═O)—(C₆-C₁₀) aryl is phenyl;
in R³:
the C₁-C₆ alkyl is selected from the group consisting of methyl, and ethyl;
the C₃-C₆ cycloalkyl is cyclopropyl;
the C₁-C₆ alkoxy is methoxy; and
the C₁-C₆ haloalkyl is trifluoromethyl;
in R²:
the four- to six-membered heterocyclyl is selected from the group consisting of azetidinyl, tetrahydrofuranyl, piperidinyl, and pyrrolidinyl, each of which is optionally substituted with 1-3 R⁴;
the C₆-C₁₀ aryl is phenyl which is optionally substituted with 1-3 R⁴;
the C₃-C₈ cycloalkyl is selected from the group consisting of cyclopropyl, cyclobutyl, and cyclopentyl, each of which is optionally substituted with 1-3 R⁴;
the five- to six-membered heteroaryl is pyrazolyl which is optionally substituted with 1-3 R⁴;
the C₁-C₆ alkyl is selected from the group consisting of methyl, ethyl, and isopropyl, which is optionally substituted with 1-3 R⁴;
the C₁-C₆ alkyl-hydroxy is hydroxymethyl; and
the C₁-C₆ alkyl-amino is —CH₂—CH₂—NH—;
in R⁴:
the R^{4a} in S(═O)₂R^{4a} is selected from the group consisting of methyl and phenyl; and
the R^{4a} in C(═O)R^{4a} is methyl;
in R⁵:
the C₁-C₆ alkyl is methyl;
in R⁶:
the C₁-C₆ alkyl is methyl; and
in R⁸:
the C₁-C₆ alkyl is methyl; and the C₁-C₆ alkoxy is methoxy.

[symbol]

In some embodiments, in Formula (IV), [symbol] is a single bond.

In some embodiments, in Formula (IV), [symbol] is a double bond.

In some embodiments, wherein in Formula (IV), [symbol] is a single bond, A is CR².

In some embodiments, wherein in Formula (IV), [symbol] is a single bond, A is CR², and n is 1.

In some embodiments, wherein in Formula (IV), [symbol] is a single bond, A is CR², and n is 0.

In some embodiments, wherein in Formula (IV), [symbol] is a single bond, A is CR², and n is 2.

In some embodiments, wherein in Formula (IV), [symbol] is a single bond, A is NR¹.

In some embodiments, wherein in Formula (IV), [symbol] is a single bond, A is NR¹, and n is 1.

In some embodiments, wherein in Formula (IV), [symbol] is a single bond, A is O.

In some embodiments, in Formula (IV), wherein [symbol] is a double bond, n is 1.

In some embodiments, wherein in Formula (IV), [symbol] is a single bond, A is CR², and n is 1,

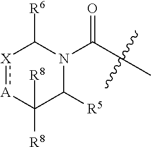

is selected from the group consisting of:

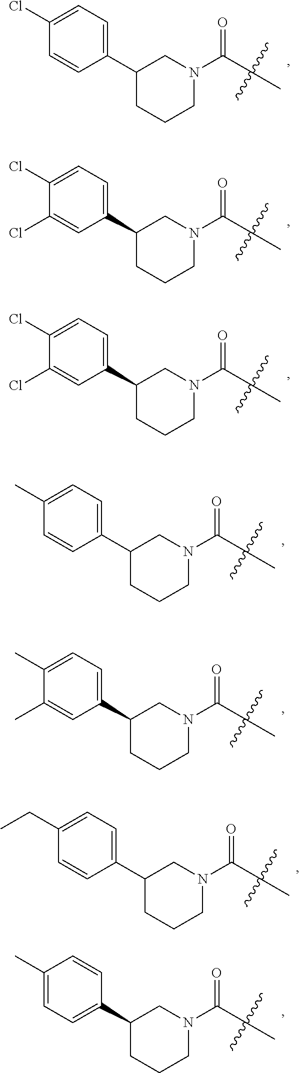

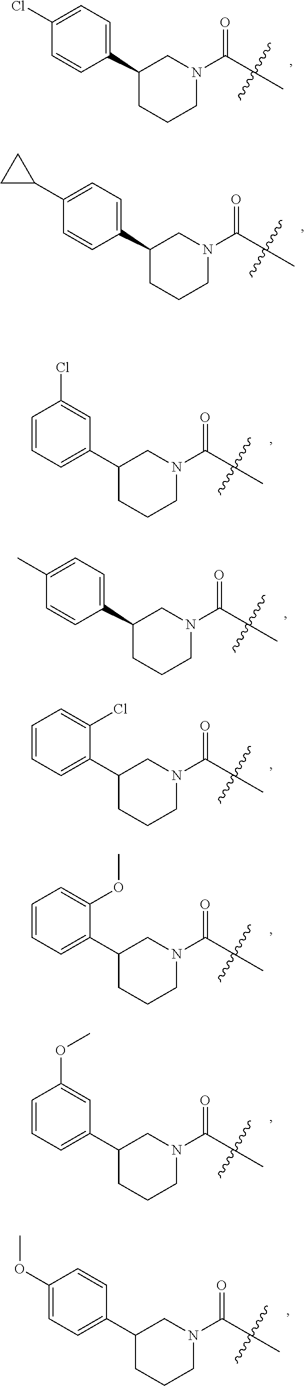

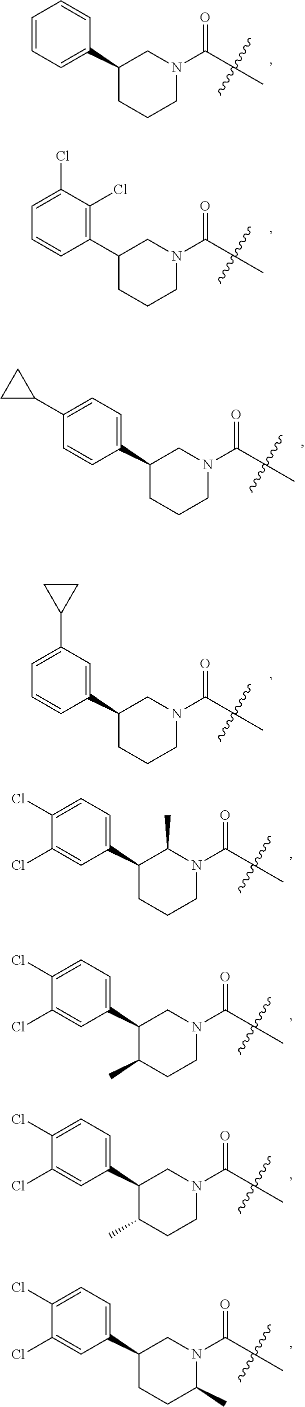

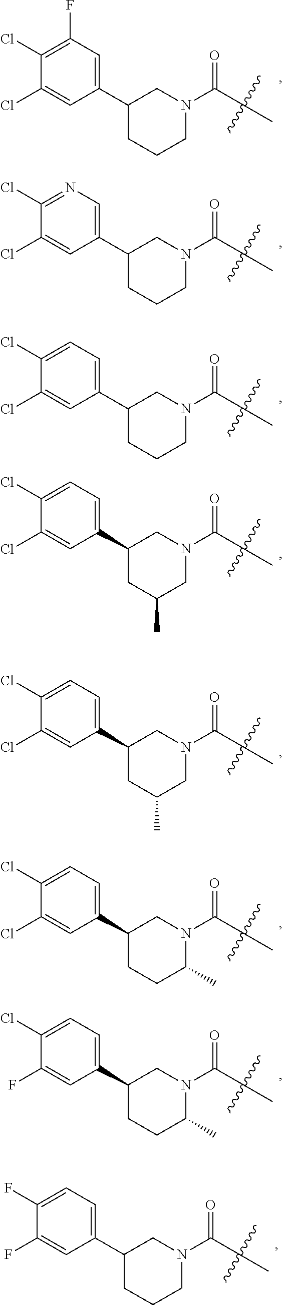

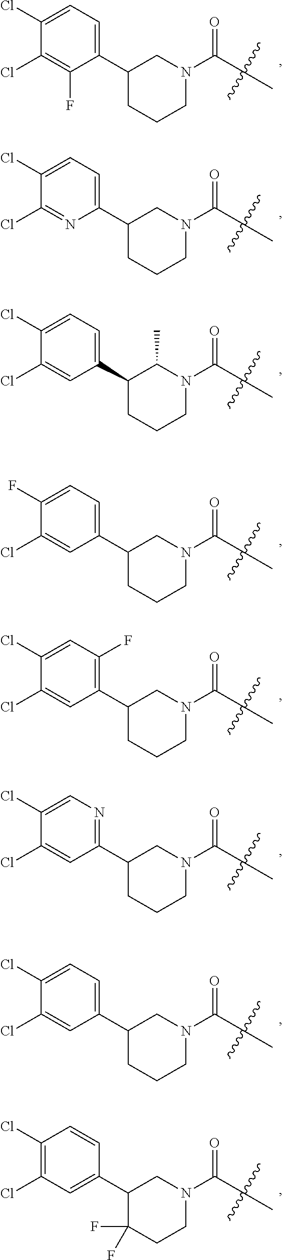

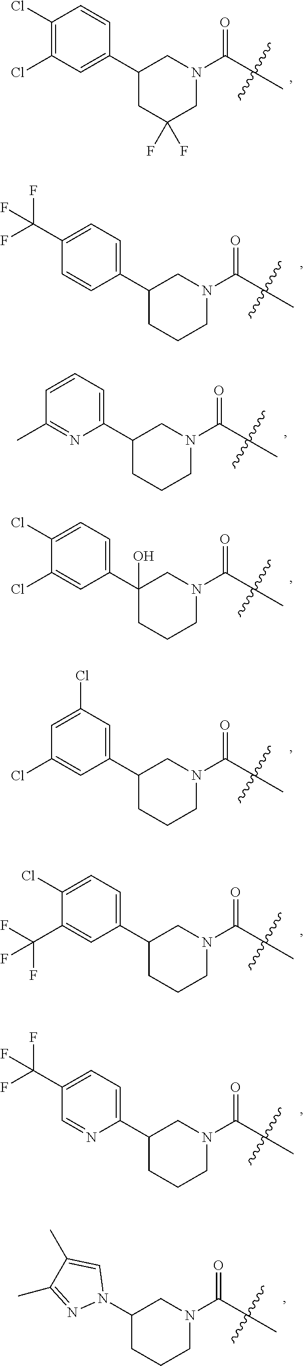

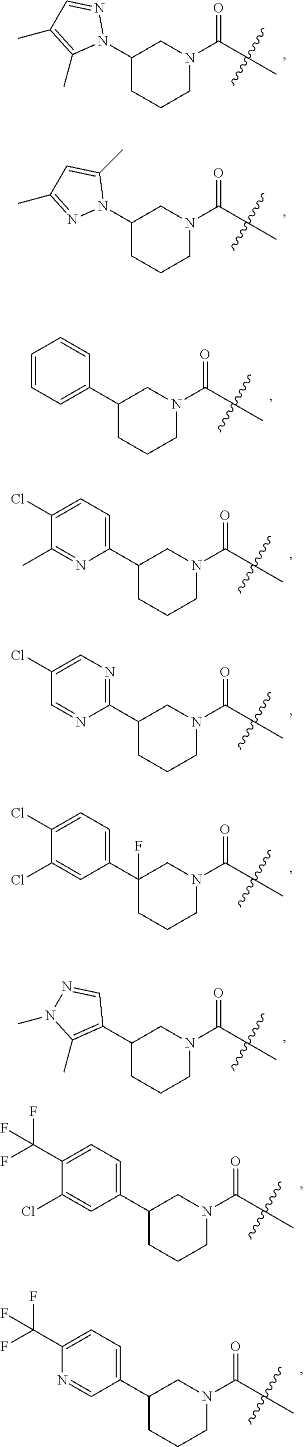

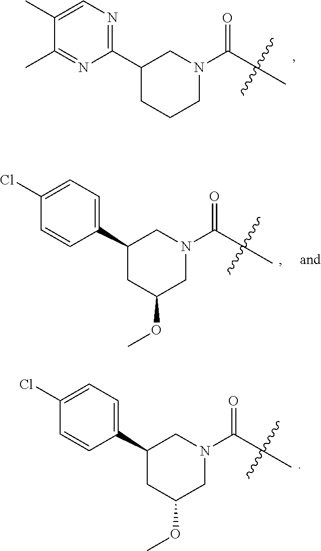 [SMILES: *C(=O)N1CCCC(c2ncc(C)c(C)n2)C1 ; *C(=O)N1C[C@@H](OC)C[C@@H](c2ccc(Cl)cc2)C1 ; *C(=O)N1C[C@H](OC)C[C@@H](c2ccc(Cl)cc2)C1]

In some embodiments, wherein in Formula (IV), [symbol] is a single bond, A is CR², and n is 0,

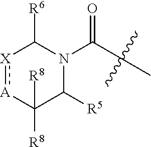

is selected from the group consisting of:

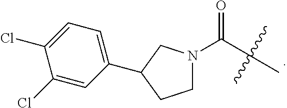 [SMILES: *C(=O)N1CCC(c2ccc(Cl)c(Cl)c2)C1]

In some embodiments, wherein in Formula (IV), [symbol] is a single bond, A is CR², and n is 2,

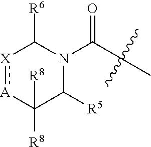

is selected from the group consisting of:

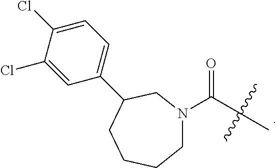 [SMILES: *C(=O)N1CCCCC(c2ccc(Cl)c(Cl)c2)C1]

In some embodiments, wherein in Formula (IV), [symbol] is a single bond, A is NR¹, and n is 1,

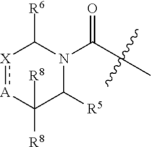

is selected from the group consisting of:

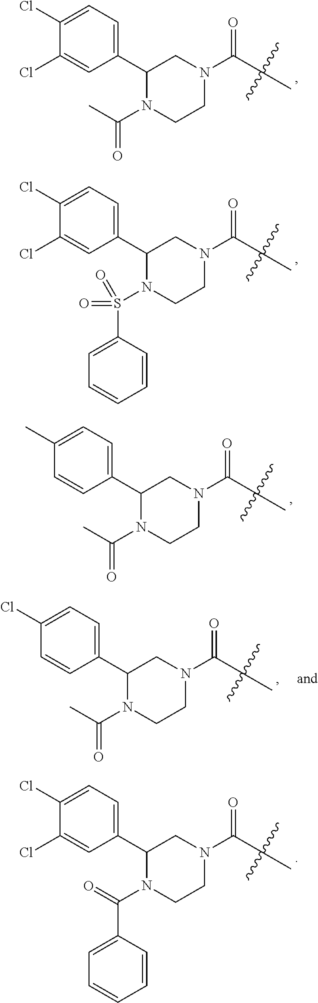

In some embodiments, wherein in Formula (IV) [symbol] is a single bond and A is O,

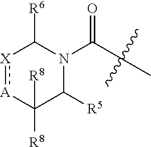

is selected from the group consisting of:

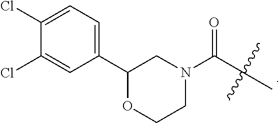 [SMILES: *C(=O)N1CCOC(c2ccc(Cl)c(Cl)c2)C1]

In some embodiments, in Formula (IV), wherein [symbol] is a double bond, n is 1,

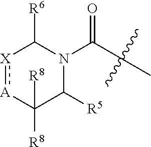

is selected from the group consisting of:

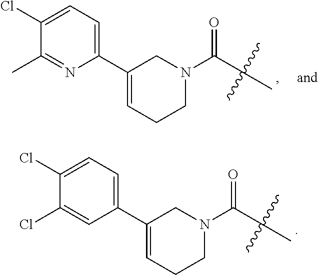 [SMILES: *C(=O)N1CCC=C(c2ccc(Cl)c(C)n2)C1 ; *C(=O)N1CCC=C(c2ccc(Cl)c(Cl)c2)C1]

### Methods of Use

In another aspect, provided herein is a method of inhibiting JAK1 comprising effecting a non-naturally occurring covalent modification at cysteine 817 as set forth in SEQ ID NO: 1, 2, 3, or 4, the modification resulting from a bond forming reaction between an electrophile and the cysteine 817 as set forth in SEQ ID NO: 1, 2, 3, or 4, wherein a sulfur atom at the cysteine residue undergoes a reaction with the electrophile.

In another aspect, provided herein is a JAK1 binding domain wherein said binding domain comprises C817 of JAK1. In some embodiments, the numbering of the amino acid positions corresponds to the amino acid position in SEQ ID NO: 1. In some embodiments the present invention provides a neoprotein comprising JAK1 protein connected through a covalent bond to a compound of Formula (I), (II), or (III) through the cysteine residue C817 of JAK1, wherein the numbering of the amino acid positions corresponds to the amino acid positions in SEQ ID NO: 1. In some aspects, the numbering of the amino acid positions corresponds to the amino acid position in SEQ ID NO: 2. In some embodiments the present invention provides a neoprotein comprising JAK1 protein connected through a covalent bond to a compound of Formula (I), (II), or (III) through the cysteine residue C817 of JAK1, wherein the numbering of the amino acid positions corresponds to the amino acid positions in SEQ ID NO: 2. In some aspects, the numbering of the amino acid positions corresponds to the amino acid position in SEQ ID NO: 3. In some embodiments the present invention provides a neoprotein comprising JAK1 protein connected through a covalent bond to a compound of Formula (I), (II), or (III) through the cysteine residue C817 of JAK1, wherein the numbering of the amino acid positions corresponds to the amino acid positions in SEQ ID NO: 3. In some aspects, the numbering of the amino acid positions corresponds to the amino acid position in SEQ ID NO: 4. In some embodiments the present invention provides a neoprotein comprising JAK1 protein connected through a covalent bond to a compound of Formula (I), (II), or (III) through the cysteine residue C817 of JAK1, wherein the numbering of the amino acid positions corresponds to the amino acid positions in SEQ ID NO: 4.

The JAK1 protein may comprise the amino acid sequence of any one of SEQ ID NOs: 1, 2, 3, or 4, or a fragment thereof. The JAK1 protein may comprise an amino acid sequence 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%, or a range defined by any two of the aforementioned percentages, identical to any one of SEQ ID NOs: 1, 2, 3, or 4, or a fragment thereof. The mutations described herein may be with regard to an amino acid sequence 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%, or a range defined by any two of the aforementioned percentages, identical to any one of SEQ ID NOs: 1, 2, 3, or 4. The JAK1 protein may comprise an amino acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identical to SEQ ID NO: 1. A mutation described herein may be in relation to an amino acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identical to SEQ ID NO: 1. The JAK1 protein may comprise an amino acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identical to SEQ ID NO: 2. A mutation described herein may be in relation to an amino acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identical to SEQ ID NO: 2. The JAK1 protein may comprise an amino acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identical to SEQ ID NO: 3. A mutation described herein may be in relation to an amino acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identical to SEQ ID NO: 3. The JAK1 protein may comprise an amino acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identical to SEQ ID NO: 4. A mutation described herein may be in relation to an amino acid sequence at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9% identical to SEQ ID NO: 4.

In some embodiments, provided herein are adducts of a compound of Formula (I), (II), or (III), or a pharmaceutically acceptable salt thereof and JAK1 that modulate binding in JAK1 protein. In some embodiments, provided herein are adducts of a compound of Formula (I), (II), or (III) and JAK1 that modulate pseudokinase domain (JH2) binding in JAK1 protein. In some embodiments, provided herein are adducts of a compound of Formula (I), (II), or (III) and JAK1 that modulate JAK1-related cytokine signaling pathways through allosteric inhibition of JAK1 binding to homo- or heterodimerization partners. In some instances, also provided herein are JAK1 ligands that bind to a cysteine in the JH2 domain. In some instances, binding to a compound of Formula (I), (II), or (III) modulates the crystal structure of JAK1. In some instances, binding to a compound of Formula (I), (II), or (III) does not inhibit JAK1 binding ATP.

In another embodiment, provided herein is a method of selectively inhibiting JAK1 over TYK2. In some embodiments, a compound of Formula (I), (II), or (III), or a pharmaceutically acceptable salt or solvate thereof is 2-fold selective for JAK1 over TYK2. In some embodiments, the compound disclosed herein is more than 2-fold selective over TYK2. In some embodiments, the compound disclosed herein is more than 5-fold selective over TYK2. In some embodiments, the compound disclosed herein is more than 8-fold selective over TYK2. In some embodiments, the compound disclosed herein is more than 10-fold selective over TYK2. In some embodiments, the compound disclosed herein is more than 20-fold selective over TYK2. In some embodiments, the compound disclosed herein is more than 50-fold selective over TYK2. In some embodiments, the compound disclosed herein is more than 100-fold selective over TYK2.

In another embodiment, disclosed herein is a method of selectively inhibiting JAK1 over one or more of JAK2 and JAK3. In some embodiments, a compound of the present invention is more than 2-fold selective over JAK2/3. In some embodiments, a compound of the present invention is more than 5-fold selective over JAK2/3. In some embodiments, a compound of the present invention is more than 10-fold selective over JAK2/3. In some embodiments, a compound of the present invention is more than 20-fold selective over JAK2/3. In some embodiments, a compound of the present invention is more than 50-fold selective over JAK2/3. In some embodiments, a compound of the present invention is more than 100-fold selective over JAK2/3.

### Combinations

In an additional embodiment the invention provides a pharmaceutical composition comprising a compound of Formula (I), (II), or (III) as defined in any of the foregoing embodiments, a pharmaceutically acceptable carrier, and a second therapeutic agent. In some embodiments, the second therapeutic agent may be selected from the group consisting of cytokine suppressive anti-inflammatory drugs, antibodies to or antagonists of other human cytokines or growth factors, IL-1, IL-2, IL-3, IL-4, IL-5, IL-6, IL-7, IL-8, IL-12, IL-15, IL-16, IL-21, IL-23, interferons, EMAP-II, GM-CSF, FGF, PDGF, CTLA or their ligands including CD154, HUMIRA™, REMICADE™, SIMPONI™ (golimumab), CIMZIA™, ACTEMRA™, CDP 571, soluble p55 or p75 TNF receptors, ENBREL™, Lenercept, TNFα converting enzyme inhibitors, IL-1 inhibitors, Interleukin 11, IL-18 antagonists, IL-12 antagonists, IL-12 antibodies, soluble IL-12 receptors, IL-12 binding proteins, non-depleting anti-CD4 inhibitors FK506, rapamycin, mycophenolate mofetil, leflunomide, NSAIDs, ibuprofen, corticosteroids, phosphodiesterase inhibitors, adensosine agonists, antithrombotic agents, complement inhibitors, adrenergic agents, IL-1β converting enzyme inhibitors, T-cell signaling kinase inhibitors, metalloproteinase inhibitors, sulfasalazine, 6-mercaptopurines, derivatives p75TNFRIgG, sIL-1RI, sIL-1RII, sIL-6R, celecoxib, hydroxychloroquine sulfate, rofecoxib, infliximab, naproxen, valdecoxib, sulfasalazine, meloxicam, acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, anti-IL15, VX-740, Roflumilast, IC-485, CDC-801, S1P1 agonists, FTY720, PKC family inhibitors, Ruboxistaurin, AEB-071, Mesopram, methotrexate, leflunomide, corticosteroids, budenoside, dexamethasone, sulfasalazine, 5-aminosalicylic acid, olsalazine, IL-1β converting enzyme inhibitors, IL-1ra, T cell signaling inhibitors, tyrosine kinase inhibitors, 6-mercaptopurines, IL-11, mesalamine, prednisone, azathioprine, mercaptopurine, infliximab, methylprednisolone sodium succinate, diphenoxylate/atrop sulfate, loperamide hydrochloride, omeprazole, folate, ciprofloxacin/dextrose-water, hydrocodone, bitartrate/apap, tetracycline hydrochloride, fluocinonide, metronidazole, thimerosal/boric acid, cholestyramine/sucrose, ciprofloxacin hydrochloride, hyoscyamine sulfate, meperidine hydrochloride, midazolam hydrochloride, oxycodone HCl/acetaminophen, promethazine hydrochloride, sodium phosphate, sulfamethoxazole/trimethoprim, polycarbophil, propoxyphene napsylate, hydrocortisone, multivitamins, balsalazide disodium, codeine phosphate/apap, colesevelam HCl, cyanocobalamin, folic acid, levofloxacin, natalizumab, interferon-gamma, methylprednisolone, azathioprine, cyclophosphamide, cyclosporine, methotrexate, 4-aminopyridine, tizanidine, interferon-β1a, AVONEX®™, interferon-(31b, BETASERON®™, interferon .alpha.-n3, interferon-.alpha., interferon-β1A-1F, Peginterferon-α2b, Copolymer 1, COPAXONE®™, hyperbaric oxygen, intravenous immunoglobulin, cladribine, cyclosporine, FK506, mycophenolate mofetil, leflunomide, NSAIDs, corticosteroids, prednisolone, phosphodiesterase inhibitors, adensosine agonists, antithrombotic agents, complement inhibitors, adrenergic agents, antiinflammatory cytokines, interferon-β, TFNβ1a, TFNβ1b, copaxone, corticosteroids, caspase inhibitors, inhibitors of caspase-1, antibodies to CD40 ligand and CD80, alemtuzumab, dronabinol, daclizumab, mitoxantrone, xaliproden hydrochloride, fampridine, glatiramer acetate, natalizumab, sinnabidol, .alpha.-immunokine NNSO3, ABR-215062, AnergiX.MS, chemokine receptor antagonists, BBR-2778, calagualine, CPI-1189, liposome encapsulated mitoxantrone, THC, CBD, cannabinoid agonists, MBP-8298, mesopram, MINA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists, interferon gamina antagonists, IL-4 agonists, diclofenac, misoprostol, naproxen, meloxicam, indomethacin, diclofenac, methotrexate, azathioprine, minocyclin, prednisone, etanercept, rofecoxib, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, risedronate sodium, sulfadiazine, thioguanine, valdecoxib, alefacept, and efalizumab, diclofenac, naproxen, ibuprofen, piroxicam, indomethacin, COX2 inhibitors, rofecoxib, valdecoxib, hydroxychloroquine, steroids, prednisolone, budenoside, dexamethasone, cytotoxics, azathioprine, cyclophosphamide, mycophenolate mofetil, inhibitors of PDE4, purine synthesis inhibitor, sulfasalazine, 5-aminosalicylic acid, olsalazine, Imuran®™, CTLA-4-IgG, anti-B7 family antibodies, anti-PD-1 family antibodies, anti-cytokine antibodies, fonotolizumab, anti-IFNg antibody, anti-receptor receptor antibodies, anti-IL-6 receptor antibody, antibodies to B-cell surface molecules, UP 394, Rituximab, anti-CD20 antibody and lymphostat-B.

The compounds of the invention are also useful in the treatment of cardiovascular disorders, such as acute myocardial infarction, acute coronary syndrome, chronic heart failure, myocardial infarction, atherosclerosis, viral myocarditis, cardiac allograft rejection, and sepsis-associated cardiac dysfunction. Furthermore, the compounds of the present invention are also useful for the treatment of central nervous system disorders such as meningococcal meningitis, Alzheimer's disease and Parkinson's disease.

In one aspect, the present invention provides a method of selectively inhibiting JAK1 in a human subject, comprising administering to the human an effective amount of a compound of Formula (I), (II), or (III) in a pharmaceutically acceptable carrier, wherein JAK1 activity is preferentially inhibited over activity of JAK2, activity of JAK3, and activity of TYK2, and less than 50%, 40%, 30%, 20%, 10%, or 5% of JAK2 and/or JAK3 activity is inhibited in the subject.

In another aspect, the invention provides a method of treating in a subject (such as an animal, or mammal, e.g., a dog, cat, or human) an autoimmune disease or disorder, or an inflammatory disease or disorder, tbc method comprising administering to the subject an effective amount of a compound of Formula (I), (IT), or (ITT) and a pharmaceutically acceptable carrier, wherein the effective amount reduces reticulocyte, NK cell, NKT cell, iNKT cell, or CD8⁺ cell count by no more than 50%, 40%, 30%, 25%, 20%, 15%, 10%, 5% relative to a pre-treatment level. In certain embodiments, more than 50%, 60%, 70%, 80%, 90%, 95%, 99% of JAK1 activity is inhibited in the subject.

In certain embodiments, the subject is in need of treatment for a condition treatable by inhibition of JAK1 activity. For example, the condition may be an inflammatory disease/disorder or an autoimmune disease/disorder, such as Rheumatoid Arthritis (RA), Crohn's disease, ankylosing spondylitis (AS), psoriatic arthritis, psoriasis, ulcerative colitis, systemic lupus erythematosus (SLE), diabetic nephropathy, dry eye syndrome. Sjogren's Syndrome, alopecia areata, vitiligo, or atopic dermatitis.

The compounds of the invention are also useful in the treatment of an ocular condition, a cancer, a solid tumor, a sarcoma, fibrosarcoma, osteomas, melanoma, retinoblastoma, a rhabdomyosarcoma, glioblastoma, neuroblastoma, teratocarcinoma, hypersensitivity reactions, hyperkinetic movement disorders, hypersensitivity pneumonitis, hypertension, hypokinetic movement disorders, aordic and peripheral aneurisms, hypothalamic-pituitary-adrenal axis evaluation, aortic dissection, arterial hypertension, arteriosclerosis, arteriovenous fistula, ataxia, spinocerebellar degenerations, structural lesions of the cerebellum, Subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, transplants, trauma/hemorrhage, type III hypersensitivity reactions, type IV hypersensitivity, unstable angina, uremia, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, heart transplant rejection, hemachromatosis, hemodialysis, hemolytic uremic syndrome/thrombolytic thrombocytopenic purpura, hemorrhage, idiopathic pulmonary fibrosis, antibody mediated cytotoxicity, Asthenia, infantile spinal muscular atrophy, inflammation of the aorta, influenza A, ionizing radiation exposure, iridocyclitis/uveitis/optic neuritis, juvenile spinal muscular atrophy, lymphoma, myeloma, leukemia, malignant ascites, hematopoietic cancers, a diabetic condition such as insulin-dependent diabetes mellitus glaucoma, diabetic retinopathy or microangiopathy, sickle cell anemia, chronic inflammation, glomerulonephritis, graft rejection, Lyme disease, von Hippel Lindau disease, pemphigoid, Paget's disease, fibrosis, sarcoidosis, cirrhosis, thyroiditis, hyperviscosity syndrome, Osler-Weber-Rendu disease, chronic occlusive pulmonary disease, asthma or edema following burns, trauma, radiation, stroke, hypoxia, ischemia, ovarian hyperstimulation syndrome, post perfusion syndrome, post pump syndrome, post-MI cardiotomy syndrome, preeclampsia, menometrorrhagia, endometriosis, pulmonary hypertension, infantile hemangioma, or infection by Herpes simplex, Herpes Zoster, human immunodeficiency virus, parapoxvirus, protozoa or toxoplasmosis, progressive supranucleo palsy, primary pulmonary hypertension, radiation therapy, Raynaud's phenomenon, Raynaud's disease, Refsum's disease, regular narrow QRS tachycardia, renovascular hypertension, restrictive cardiomyopathy, sarcoma, senile chorea, senile dementia of Lewy body type, shock, skin allograft, skin changes syndrome, ocular or macular edema, ocular neovascular disease, scleritis, radial keratotomy, uveitis, vitritis, myopia, optic pits, chronic retinal detachment, post-laser treatment complications, conjunctivitis, Stargardt's disease, Eales disease, retinopathy, macular degeneration, restenosis, ischemia/reperfusion injury, ischemic stroke, vascular occlusion, carotid obstructive disease, ulcerative colitis, inflammatory bowel disease, diabetes, diabetes mellitus, insulin dependent diabetes mellitus, allergic diseases, dermatitis scleroderma, graft versus host disease, organ transplant rejection (including but not limited to bone marrow and solid organ rejection), acute or chronic immune disease associated with organ transplantation, sarcoidosis, disseminated intravascular coagulation, Kawasaki's disease, nephrotic syndrome, chronic fatigue syndrome, Wegener's granulomatosis, Henoch-Schoenlein purpurea, microscopic vasculitis of the kidneys, chronic active hepatitis, septic shock, toxic shock syndrome, sepsis syndrome, cachexia, infectious diseases, parasitic diseases, acquired immunodeficiency syndrome, acute transverse myelitis, Huntington's chorea, stroke, primary biliary cirrhosis, hemolytic anemia, malignancies, Addison's disease, idiopathic Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune hemolytic anemia, Coombs positive hemolytic anemia, acquired pernicious anemia, juvenile pernicious anemia, peripheral vascular disorders, peritonitis, pernicious anemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, His bundle arrythmias, HIV infection/HIV neuropathy, common varied immunodeficiency (common variable hypogammaglobulinemia), dilated cardiomyopathy, female infertility, ovarian failure, premature ovarian failure, fibrotic lung disease, chronic wound healing, cryptogenic fibrosing alveolitis, post-inflammatory interstitial lung disease, interstitial pneumonitis, Pneumocystis carinii pneumonia, pneumonia, connective tissue disease associated interstitial lung disease, mixed connective tissue disease, associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycaemia, type B insulin resistance with Acanthosis nigricans, hypoparathyroidism, acute immune disease associated with organ transplantation, chronic immune disease associated with organ transplantation, osteoarthritis, primary sclerosing cholangitis, psoriasis type 1, psoriasis type 2, idiopathic leucopaenia, autoimmune neutropenia, renal disease NOS, glomerulonephritides, microscopic vasculitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, acute and chronic pain (different forms of pain), Goodpasture's syndrome, pulmonary manifestation of polyarteritis nodosa, acute rheumatic fever, rheumatoid spondylitis, Still's disease, systemic sclerosis, Sjogren's syndrome, Takayasu's disease/arteritis, autoimmune thrombocytopenia, toxicity, transplants, and diseases involving inappropriate vascularization for example diabetic retinopathy, retinopathy of prematurity, choroidal neovascularization due to age-related macular degeneration, and infantile hemangiomas in human beings. In addition, such compounds may be useful in the treatment of disorders such as ascites, effusions, and exudates, including for example macular edema, cerebral edema, acute lung injury, adult respiratory distress syndrome (ARDS), proliferative disorders such as restenosis, fibrotic disorders such as hepatic cirrhosis and atherosclerosis, mesangial cell proliferative disorders such as diabetic nephropathy, malignant nephrosclerosis, thrombotic microangiopathy syndromes, and glomerulopathies, myocardial angiogenesis, coronary and cerebral collaterals, ischemic limb angiogenesis, ischemia/reperfusion injury, peptic ulcer Helicobacter related diseases, virally-induced angiogenic disorders, preeclampsia, menometrorrhagia, cat scratch fever, rubeosis, neovascular glaucoma and retinopathies such as those associated with diabetic retinopathy, retinopathy of prematurity, or age-related macular degeneration. In addition, these compounds can be used as active agents against hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

Compounds of Formula (I), (II), and (Ill) can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the compound of the present invention. The additional agent also can be an agent that imparts a beneficial attribute to the therapeutic composition e.g., an agent that affects the viscosity of the composition.

It should further be understood that the combinations which are to be included within this invention are those combinations useful for their intended purpose. The agents set forth below are illustrative for purposes and not intended to be limited. The combinations, which are part of this invention, can be the compounds of the present invention and at least one additional agent selected from the lists below. The combination can also include more than one additional agent, e.g., two or three additional agents if the combination is such that the formed composition can perform its intended function.

Preferred combinations are non-steroidal anti-inflammatory drug(s) also referred to as NSAIDS which include drugs like ibuprofen. Other preferred combinations are corticosteroids including prednisolone; the well-known side-effects of steroid use can be reduced or even eliminated by tapering the steroid dose required when treating patients in combination with the compounds of this invention. Non-limiting examples of therapeutic agents for rheumatoid arthritis with which a compound of Formula (I), (II), or (III) of the invention can be combined include the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-12, IL-15, IL-16, IL-21, IL-23, interferons, EMAP-II, GM-CSF, FGF, and PDGF. Compounds of the invention can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7 (U.S. Pat. No. 6,090,382, HUMIRA™), CA2 (REMICADE™), SIMPONI™ (golimumab), CIMZIA™, ACTEMRA™, CDP 571, and soluble p55 or p75 TNF receptors, derivatives, thereof, (p75TNFR1gG (ENBREL™) or p55TNFR1gG (Lenercept), and also TNF.alpha. converting enzyme (TACE) inhibitors; similarly, IL-1 inhibitors (Interleukin-1-converting enzyme inhibitors, IL-IRA etc.) may be effective for the same reason. Other preferred combinations include Interleukin 11. Yet other preferred combinations are the other key players of the autoimmune response which may act parallel to, dependent on or in concert with IL-18 function; especially preferred are IL-12 antagonists including IL-12 antibodies or soluble IL-12 receptors, or IL-12 binding proteins. It has been shown that IL-12 and IL-18 have overlapping but distinct functions and a combination of antagonists to both may be most effective. Yet another preferred combination is non-depleting anti-CD4 inhibitors. Yet other preferred combinations include antagonists of the co-stimulatory pathway CD80 (B7.1) or CD86 (B7.2) including antibodies, soluble receptors or antagonistic ligands.

A compound of Formula (I), (II), or (III), or a pharmaceutically acceptable salt or solvate thereof may also be combined with agents, such as methotrexate, 6-mercaptopurine, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, kctotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNF alpha, quadrature, or IL-1 (e.g., NIK, IKK, p38 or MAP kinase inhibitors), IL-1β converting enzyme inhibitors, T-cell signaling inhibitors such as kinase inhibitors, metalloproteinase inhibitors, sulfasalazine, 6-mercaptopurines, angiotensin converting enzyme inhibitors, soluble cytokine receptors and derivatives thereof (e.g. soluble p55 or p75 TNF receptors and the derivatives p75TNFRIgG (Enbrel™) and p55TNFRIgG (Lenercept), sIL-1RI, sIL-1RII, sIL-6R), anti-inflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-TG, IL-18 BP, anti-IL-12, Anti-IL15, BIRB-796, SCIO-469, VX-702, AMG-548, VX-740, Roflumilast, IC-485, CDC-801, SIP1 agonists (such as FTY720), PKC family inhibitors (such as Ruboxistaurin or AEB-071) and Mesopram. Preferred combinations include methotrexate or leflunomide and in moderate or severe rheumatoid arthritis cases, cyclosporine and anti-TNF antibodies as noted above.

Non-limiting examples of therapeutic agents for inflammatory bowel disease with which a compound of Formula (I), (II), or (III), or a pharmaceutically acceptable salt or solvate thereof, can be combined include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, IL-23, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signaling by proinflammatory cytokines such as TNFα or IL-1 (e.g. NIK, IKK, or MAP kinase inhibitors); IL-1β converting enzyme inhibitors; TNFα converting enzyme inhibitors; T-cell signaling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-11, IL-1D and TGFβ). Preferred examples of therapeutic agents for Crohn's disease with which a compound of Formula (I), (II), or (III) can be combined include the following: TNF antagonists, for example, anti-TNF antibodies, D2E7 (U.S. Pat. No. 6,090,382, HUMIRA™), CA2 (REMICADE™), CDP 571, TNFR-Ig constructs, (p75TNFRIgG (ENBREL™) and p55TNFRIgG (LENERCEPT™) inhibitors and PDE4 inhibitors. A compound of Formula (I), (II), or (III) can be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1B converting enzyme inhibitors and IL-1ra; T cell signaling inhibitors, for example, tyrosine kinase inhibitors; 6-mercaptopurine; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis with which a compound of Formula (I), (II), or (III) can be combined include the following: corticosteroids; prednisolone; methylprednisolone; azathioprine; cyclophosphamide; cyclosporine; methotrexate; 4-aminopyridine; tizanidine; interferon-301a (AVONEX®™; Biogen); interferon-β1b (BETASERON®™; Chiron/Berlex); interferon .alpha.-n3) (Interferon Sciences/Fujimoto), interferon-a (Alfa Wassermann/J&J), interferon β1A-1F (Serono/Inhale Therapeutics), Peginterferon alpha. 2b (Enzon/Schering-Plough), Copolymer 1 (Cop-1; COPAXONE®™; Teva Pharmaceutical Industries, Inc.); hyperbaric oxygen; intravenous immunoglobulin; cladribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of Formula (I), (II), or (III) can be combined with antibodies to cell surface molecules such as CD2, CD3, CD4, CD8, CD19, CD20, CD25, CD28, CD30, CD40, CD45, CD69, CD80, CD86, CD90 or their ligands. A compound of Formula (I), (II), or (III) may also be combined with agents such as methotrexate, cyclosporine, FK506, rapamycin, mycophenolate mofetil, leflunomide, an SIP1 agonist, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNF.alpha..quadrature. or IL-1 (e.g., NIK, IKK, p38 or MAP kinase inhibitors), IL-10 converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-1β and TGFβ).

Preferred examples of therapeutic agents for multiple sclerosis in which a compound of Formula (I) or (II) can be combined to include interferon-β, for example, IFNβ1a and IFNβ1b; copaxone, corticosteroids, caspase inhibitors, for example inhibitors of caspase-1, IL-1 inhibitors, TNF inhibitors, and antibodies to CD40 ligand and CD80.

A compound of Formula (I), (II), or (III), or a pharmaceutically acceptable salt or solvate thereof, may also be combined with agents, such as alemtuzumab, dronabinol, daclizumab, mitoxantrone, xaliproden hydrochloride, fampridine, glatiramer acetate, natalizumab, sinnabidol, α-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Non-limiting examples of therapeutic agents for ankylosing spondylitis with which a compound of Formula (I), (II), or (III), or a pharmaceutically acceptable salt or solvate thereof, can be combined include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, and anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™)

Non-limiting examples of therapeutic agents for asthma with which a compound of Formula (I), (II), or (III), or a pharmaceutically acceptable salt or solvate thereof, can be combined include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine HCl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone, anti-IL-1β antibody, and metaproterenol sulfate.

Non-limiting examples of therapeutic agents for COPD with which a compound of Formula (I), (II), or (III), or a pharmaceutically acceptable salt or solvate thereof, can be combined include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, cilomilast and roflumilast.

Non-limiting examples of therapeutic agents for HCV with which a compound of Formula (I), (II), or (III), or a pharmaceutically acceptable salt or solvate thereof, can be combined include the following: Interferon-alpha-2.alpha., Interferon-alpha-2β, Interferon-alpha con1, Interferon-alpha-nl, pegylated interferon-alpha-2.alpha., pegylated interferon-alpha-2β, ribavirin, peginterferon alfa-2b+ribavirin, ursodeoxycholic acid, glycyrrhizic acid, thymalfasin, Maxamine, VX-497 and any compounds that are used to treat HCV through intervention with the following targets: HCV polymerase, HCV protease, HCV helicase, and HCV IRES (internal ribosome entry site).

Non-limiting examples of therapeutic agents for Idiopathic Pulmonary Fibrosis with which a compound of Formula (I), (II), or (III), or a pharmaceutically acceptable salt or solvate thereof, can be combined include the following: prednisone, azathioprine, albuterol, colchicine, albuterol sulfate, digoxin, gamma interferon, methylprednisolone sodium succinate, lorazepam, furosemide, lisinopril, nitroglycerin, spironolactone, cyclophosphamide, ipratropium bromide, actinomycin d, alteplase, fluticasone propionate, levofloxacin, metaproterenol sulfate, morphine sulfate, oxycodone HCl, potassium chloride, triamcinolone acetonide, tacrolimus anhydrous, calcium, interferon-alpha, methotrexate, mycophenolate mofetil and interferon-gamma-1.

Non-limiting examples of therapeutic agents for myocardial infarction with which a compound of Formula (I), (II), or (III), or a pharmaceutically acceptable salt or solvate thereof, can be combined include the following: aspirin, nitroglycerin, metoprolol tartrate, enoxaparin sodium, heparin sodium, clopidogrel bisulfate, carvedilol, atenolol, morphine sulfate, metoprolol succinate, warfarin sodium, lisinopril, isosorbide mononitrate, digoxin, furosemide, simvastatin, ramipril, tenecteplase, enalapril maleate, torsemide, retavase, losartan potassium, quinapril hydrochloride/magnesium carbonate, bumetanide, alteplase, enalaprilat, amiodarone hydrochloride, tirofiban HCl m-hydrate, diltiazem hydrochloride, captopril, irbesartan, valsartan, propranolol hydrochloride, fosinopril sodium, lidocaine hydrochloride, eptifibatide, cefazolin sodium, atropine sulfate, aminocaproic acid, spironolactone, interferon, sotalol hydrochloride, potassium chloride, docusate sodium, dobutamine HCl, alprazolam, pravastatin sodium, atorvastatin calcium, midazolam hydrochloride, meperidine hydrochloride, isosorbide dinitrate, epinephrine, dopamine hydrochloride, bivalirudin, rosuvastatin, ezetimibe/simvastatin, avasimibe, and cariporide.

Non-limiting examples of therapeutic agents for psoriasis with which a compound of Formula (I), (II), or (III), or a pharmaceutically acceptable salt or solvate thereof, can be combined include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, hc/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine, ABT-874 and ustekinamab.

Non-limiting examples of therapeutic agents for psoriatic arthritis with which a compound of Formula (I), (II), or (III), or a pharmaceutically acceptable salt or solvate thereof, can be combined include the following: methotrexate, etanercept, rofecoxib, colecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, D2E7 (U.S. Pat. No. 6,090,382, HUMIRA™), and efalizumab.

Non-limiting examples of therapeutic agents for restenosis with which a compound of Formula (I), (II), or (III), or a pharmaceutically acceptable salt or solvate thereof, can be combined include the following: sirolimus, paclitaxel, everolimus, tacrolimus, ABT-578, and acetaminophen.

Non-limiting examples of therapeutic agents for sciatica with which a compound of Formula (I), (II), or (III), or a pharmaceutically acceptable salt or solvate thereof, can be combined include the following: hydrocodone bitartrate/apap, rofecoxib, cyclobenzaprine HCl, methylprednisolone, naproxen, ibuprofen, oxycodone HCl/acetaminophen, colecoxib, valdecoxib, methylprednisolone acetate, prednisone, codeine phosphate/apap, tramadol hel/acetaminophen, metaxalone, meloxicam, methocarbamol, lidocaine hydrochloride, diclofenac sodium, gabapentin, dexamethasone, carisoprodol, ketorolac tromethamine, indomethacin, acetaminophen, diazepam, nabumetone, oxycodone HCl, tizanidine HCl, diclofenac sodium/misoprostol, propoxyphene n-pap, asa/oxycod/oxycodone ter, ibuprofen/hydrocodone bit, tramadol HCl, etodolac, propoxyphene HCl, amitriptyline HCl, cansoprodol/codeine phos/asa, morphine sulfate, multivitamins, naproxen sodium, orphenadrine citrate, and temazepam.

Preferred examples of therapeutic agents for SLE (Lupus) with which a compound of Formula (I), (II), or (III), or a pharmaceutically acceptable salt or solvate thereof, can be combined include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®™ A compound of Formula (I), (II), or (III), or a pharmaceutically acceptable salt or solvate thereof, may also be combined with agents such as sulfasalazine, 5-aminosalicylic acid, olsalazine, Imuran®™ and agents which interfere with synthesis, production or action of proinflammatory cytokines such as IL-1, for example, caspase inhibitors like IL-1β converting enzyme inhibitors and IL-1ra. A compound of Formula (I), (II), or (III), or a pharmaceutically acceptable salt or solvate thereof, may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of Formula (I), (II), or (III), or a pharmaceutically acceptable salt or solvate thereof, can be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of Formula (I), (II), or (III), or a pharmaceutically acceptable salt or solvate thereof, may also be used with LJP 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (U.S. Pat. No. 6,090,382; HUMIRA™), CA2 (REMICADE™), CDP 571, TNFR-Ig constructs, (p75TNFRIgG (ENBREL™) and p55TNFRIgG (LENERCEPT™).

### Further Forms of Compounds

In one aspect, the compound of Formula (I), (II), or (III), or a pharmaceutically acceptable salt or solvate thereof, possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In one aspect, stereoisomers are obtained by stereoselective synthesis.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

Compounds described herein may be formed as, and/or used as, acceptable salts. The type of acceptable salts, include, but are not limited to: (1) acid addition salts, formed by reacting the free base form of the compound with an acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Ligand

In some embodiments, a ligand competes with a probe compound described herein for binding with a reactive lysine residue. In some embodiments, a ligand competes with a probe compound described herein for binding with a cysteine residue. In some instances, a ligand comprises a small molecule compound, a polynucleotide, a polypeptide or its fragments thereof, or a peptidomimetic. In some embodiments, the ligand comprises a small molecule compound. In some instances, a small molecule compound comprises a fragment moiety that facilitate interaction of the compound with a reactive lysine residue. In some cases, a small molecule compound comprises a small molecule fragment that facilitates hydrophobic interaction, hydrogen bonding, or a combination thereof.

In some embodiments, a ligand comprises a polynucleotide. In some instances, the polynucleotide comprises an endogenous substrate that interacts with a lysine-containing protein. In some instances, the polynucleotide comprises modified and/or synthetic substrate. In some cases, the polynucleotide comprises natural nucleotides. In other cases, the polynucleotide comprises artificial nucleotides.

In some instances, a polynucleotide comprises from about 8 to about 50 bases in length. In some cases, a polynucleotide comprises from about 12 to about 45, from about 15 to about 40, from about 20 to about 40, or from about 25 to about 30 bases in length. In some cases, a polynucleotide comprises 8, 9, 10, 11, 12, 13, 14, 15, 16, 17, 18, 19, 20, 21, 22, 23, 24, 25, 30, 35, 40, 45, or 50 bases in length.

In some embodiments, a ligand comprises a polypeptide or its fragments thereof. In some instances, the polypeptide comprises a wild-type functional protein, protein variants, or mutants that are substrates for a lysine-containing protein of interest. In some instances, fragments of the polypeptide comprise truncated functional proteins that interact with the lysine-containing protein of interest.

In some instances, a functional fragment of a polypeptide comprises from about 10 to about 80 amino acid residues in length. In some instances, the functional fragment comprises from about 15 to about 70, from about 20 to about 60, from about 30 to about 50, or from about 40 to about 80 amino acid residues in length. In some cases, the functional fragment comprises about 10, 15, 20, 25, 30, 35, 40, 45, 50, 55, 60, 70, 80, or more amino acid residues in length.

In some cases, a polypeptide or its fragments thereof comprise natural amino acids, unnatural amino acids, or a combination thereof. In some cases, the polypeptide or its fragments thereof comprise L-amino acids, D-amino acids, or a combination thereof.

In some instances, a ligand comprises a peptidomimetic. Peptidomimetic is a small protein-like chain that mimics a peptide. Exemplary peptidomimetics include, but are not limited to, peptoids, β-peptides, or foldamers. Peptoids, also known as poly-N-substituted glycines, are a class of peptidomimetics in which the side chains are appended to the nitrogen atom of the peptide backbone instead of the α-carbon. β-peptides are β-amino acids in which the amino groups are bonded to the β-carbon rather than the α-carbon. A foldamer is a discrete chain molecule or oligomer that folds into an ordered conformation such as helices and β-sheets.

As referred to above, exemplary unnatural amino acid residues comprise, for example, amino acid analogs such as D-amino acid analogs; racemic analogs; or analogs of amino acid residue alanine, valine, glycine, leucine, arginine, lysine, aspartic acid, glutamic acid, cysteine, methionine, tyrosine, phenylalanine, tryptophan, serine, threonine, or proline. Exemplary β-amino acid analogs include, but are not limited to, cyclic β-amino acid analogs, β-alanine, (R)-β-phenylalanine, (R)-1,2,3,4-tetrahydro-isoquinoline-3-acetic acid, (R)-3-amino-4-(1-naphthyl)-butyric acid, (R)-3-amino-4-(2,4-dichlorophenyl)butyric acid, (R)-3-amino-4-(2-chlorophenyl)-butyric acid, (R)-3-amino-4-(2-cyanophenyl)-butyric acid, (R)-3-amino-4-(2-fluorophenyl)-butyric acid, (R)-3-amino-4-(2-furyl)-butyric acid, (R)-3-amino-4-(2-methylphenyl)-butyric acid, (R)-3-amino-4-(2-naphthyl)-butyric acid, (R)-3-amino-4-(2-thienyl)-butyric acid, (R)-3-amino-4-(2-trifluoromethylphenyl)-butyric acid, (R)-3-amino-4-(3,4-dichlorophenyl)butyric acid, (R)-3-amino-4-(3,4-difluorophenyl)butyric acid, (R)-3-amino-4-(3-benzothienyl)-butyric acid, (R)-3-amino-4-(3-chlorophenyl)-butyric acid, (R)-3-amino-4-(3-cyanophenyl)-butyric acid, (R)-3-amino-4-(3-fluorophenyl)-butyric acid, (R)-3-amino-4-(3-methylphenyl)-butyric acid, (R)-3-amino-4-(3-pyridyl)-butyric acid, (R)-3-amino-4-(3-thienyl)-butyric acid, (R)-3-amino-4-(3-trifluoromethylphenyl)-butyric acid, (R)-3-amino-4-(4-bromophenyl)-butyric acid, (R)-3-amino-4-(4-chlorophenyl)-butyric acid, (R)-3-amino-4-(4-cyanophenyl)-butyric acid, (R)-3-amino-4-(4-fluorophenyl)-butyric acid, (R)-3-amino-4-(4-iodophenyl)-butyric acid, (R)-3-amino-4-(4-methylphenyl)-butyric acid, (R)-3-amino-4-(4-nitrophenyl)-butyric acid, (R)-3-amino-4-(4-pyridyl)-butyric acid, (R)-3-amino-4-(4-trifluoromethylphenyl)-butyric acid, (R)-3-amino-4-pentafluoro-phenylbutyric acid, (R)-3-amino-5-hexenoic acid, (R)-3-amino-5-hexynoic acid, (R)-3-amino-5-phenylpentanoic acid, (R)-3-amino-6-phenyl-5-hexenoic acid, (S)-1,2,3,4-tetrahydro-isoquinoline-3-acetic acid, (S)-3-amino-4-(1-naphthyl)-butyric acid, (S)-3-amino-4-(2,4-dichlorophenyl)butyric acid, (S)-3-amino-4-(2-chlorophenyl)-butyric acid, (S)-3-amino-4-(2-cyanophenyl)-butyric acid, (S)-3-amino-4-(2-fluorophenyl)-butyric acid, (S)-3-amino-4-(2-furyl)-butyric acid, (S)-3-amino-4-(2-methylphenyl)-butyric acid, (S)-3-amino-4-(2-naphthyl)-butyric acid, (S)-3-amino-4-(2-thienyl)-butyric acid, (S)-3-amino-4-(2-trifluoromethylphenyl)-butyric acid, (S)-3-amino-4-(3,4-dichlorophenyl)butyric acid, (S)-3-amino-4-(3,4-difluorophenyl)butyric acid, (S)-3-amino-4-(3-benzothienyl)-butyric acid, (S)-3-amino-4-(3-chlorophenyl)-butyric acid, (S)-3-amino-4-(3-cyanophenyl)-butyric acid, (S)-3-amino-4-(3-fluorophenyl)-butyric acid, (S)-3-amino-4-(3-methylphenyl)-butyric acid, (S)-3-amino-4-(3-pyridyl)-butyric acid, (S)-3-amino-4-(3-thienyl)-butyric acid, (S)-3-amino-4-(3-trifluoromethylphenyl)-butyric acid, (S)-3-amino-4-(4-bromophenyl)-butyric acid, (S)-3-amino-4-(4-chlorophenyl) butyric acid, (S)-3-amino-4-(4-cyanophenyl)-butyric acid, (S)-3-amino-4-(4-fluorophenyl) butyric acid, (S)-3-amino-4-(4-iodophenyl)-butyric acid, (S)-3-amino-4-(4-methylphenyl)-butyric acid, (S)-3-amino-4-(4-nitrophenyl)-butyric acid, (S)-3-amino-4-(4-pyridyl)-butyric acid, (S)-3-amino-4-(4-trifluoromethylphenyl)-butyric acid, (S)-3-amino-4-pentafluoro-phenylbutyric acid, (S)-3-amino-5-hexenoic acid, (S)-3-amino-5-hexynoic acid, (S)-3-amino-5-phenylpentanoic acid, (S)-3-amino-6-phenyl-5-hexenoic acid, 1,2,5,6-tetrahydropyridine-3-carboxylic acid, 1,2,5,6-tetrahydropyridine-4-carboxylic acid, 3-amino-3-(2-chlorophenyl)-propionic acid, 3-amino-3-(2-thienyl)-propionic acid, 3-amino-3-(3-bromophenyl)-propionic acid, 3-amino-3-(4-chlorophenyl)-propionic acid, 3-amino-3-(4-methoxyphenyl)-propionic acid, 3-amino-4,4,4-trifluoro-butyric acid, 3-aminoadipic acid, D-β-phenylalanine, β-leucine, L-β-homoalanine, L-β-homoaspartic acid γ-benzyl ester, L-β-homoglutamic acid δ-benzyl ester, L-β-homoisoleucine, L-β-homoleucine, L-β-homomethionine, L-β-homophenylalanine, L-β-homoproline, L-β-homotryptophan, L-β-homovaline, L-Nω-benzyloxycarbonyl-β-homolysine, Nω-L-β-homoarginine, O-benzyl-L-β-homohydroxyproline, O-benzyl-L-β-homoserine, O-benzyl-L-β-homothreonine, O-benzyl-L-β-homotyrosine, γ-trityl-L-β-homoasparagine, (R)-β-phenylalanine, L-β-homoaspartic acid γ-t-butyl ester, L-β-homoglutamic acid δ-t-butyl ester, L-Nω-β-homolysine, Nδ-trityl-L-β-homoglutamine, Nω-2,2,4,6,7-pentamethyl-dihydrobenzofuran-5-sulfonyl-L-β-homoarginine, O-t-butyl-L-R-homohydroxy-proline, O-t-butyl-L-β-homoserine, O-t-butyl-L-β-homothreonine, O-t-butyl-L-β-homotyrosine, 2-aminocyclopentane carboxylic acid, and 2-aminocyclohexane carboxylic acid.

In some instances, unnatural amino acid residues comprise a racemic mixture of amino acid analogs. For example, in some instances, the D isomer of the amino acid analog is used. In some cases, the L isomer of the amino acid analog is used. In some instances, the amino acid analog comprises chiral centers that are in the R or S configuration. Sometimes, the amino group(s) of a β-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a β-amino acid analog is protected, e.g., as its ester derivative. In some cases, the salt of the amino acid analog is used.

In some cases, unnatural amino acid residues comprise analogs of amino acid residue alanine, valine, glycine, leucine, arginine, lysine, aspartic acid, glutamic acid, cysteine, methionine, tyrosine, phenylalanine, tryptophan, serine, threonine, or proline. Exemplary amino acid analogs of alanine, valine, glycine, and leucine include, but are not limited to, α-methoxyglycine, α-allyl-L-alanine, α-aminoisobutyric acid, α-methyl-leucine, β-(1-naphthyl)-D-alanine, β-(1-naphthyl)-L-alanine, β-(2-naphthyl)-D-alanine, β-(2-naphthyl)-L-alanine, β-(2-pyridyl)-D-alanine, β-(2-pyridyl)-L-alanine, β-(2-thienyl)-D-alanine, β-(2-thienyl)-L-alanine, β-(3-benzothienyl)-D-alanine, β-(3-benzothienyl)-L-alanine, β-(3-pyridyl)-D-alanine, β-(3-pyridyl)-L-alanine, β-(4-pyridyl)-D-alanine, β-(4-pyridyl)-L-alanine, β-chloro-L-alanine, β-cyano-L-alanine, β-cyclohexyl-D-alanine, β-cyclohexyl-L-alanine, β-cyclopenten-1-yl-alanine, β-cyclopentyl-alanine, β-cyclopropyl-L-Ala-OH dicyclohexylammonium salt, β-t-butyl-D-alanine, β-t-butyl-L-alanine, γ-aminobutyric acid, L-α,β-diaminopropionic acid, 2,4-dinitro-phenylglycine, 2,5-dihydro-D-phenylglycine, 2-amino-4,4,4-trifluorobutyric acid, 2-fluoro-phenylglycine, 3-amino-4,4,4-trifluoro-butyric acid, 3-fluoro-valine, 4,4,4-trifluoro-valine, 4,5-dehydro-L-leu-OH.dicyclohexylammonium salt, 4-fluoro-D-phenylglycine, 4-fluoro-L-phenylglycine, 4-hydroxy-D-phenylglycine, 5,5,5-trifluoro-leucine, 6-aminohexanoic acid, cyclopentyl-D-Gly-OH dicyclohexylammonium salt, cyclopentyl-Gly-OH dicyclohexylammonium salt, D-α,β-diaminopropionic acid, D-α-aminobutyric acid, D-α-t-butylglycine, D-(2-thienyl)glycine, D-(3-thienyl)glycine, D-2-aminocaproic acid, D-2-indanylglycine, D-allylglycine-dicyclohexylammonium salt, D-cyclohexylglycine, D-norvaline, D-phenylglycine, β-aminobutyric acid, β-aminoisobutyric acid, (2-bromophenyl)glycine, (2-methoxyphenyl)glycine, (2-methylphenyl)glycine, (2-thiazoyl)glycine, (2-thienyl)glycine, 2-amino-3-(dimethylamino)-propionic acid, L-α,β-diaminopropionic acid, L-α-aminobutyric acid, L-α-t-butylglycine, L-(3-thienyl)glycine, L-2-amino-3-(dimethylamino)-propionic acid, L-2-aminocaproic acid dicyclohexylammonium salt, L-2-indanylglycine, L-allylglycine.dicyclohexyl ammonium salt, L-cyclohexylglycine, L-phenylglycine, L-propargylglycine, L-norvaline, N-α-aminomethyl-L-alanine, D-α,γ-diaminobutyric acid, L-α,γ-diaminobutyric acid, β-cyclopropyl-L-alanine, (N-β-(2,4-dinitrophenyl))-L-α,β-diaminopropionic acid, (N-β-1-(4,4-dimethyl-2,6-dioxocyclohex-1-ylidene)ethyl)-D-α,β-diaminopropionic acid, (N-β-1-(4,4-dimethyl-2,6-dioxocyclohex-1-ylidene)ethyl)-L-α,β-diaminopropionic acid, (N-β-4-methyltrityl)-L-α,β-diaminopropionic acid, (N-β-allyloxycarbonyl)-L-α,β-diaminopropionic acid, (N-γ-1-(4,4-dimethyl-2,6-dioxocyclohex-1-ylidene)ethyl)-D-α,γ-diaminobutyric acid, (N-γ-1-(4,4-dimethyl-2,6-dioxocyclohex-1-ylidene)ethyl)-L-α,γ-diaminobutyric acid, (N-γ-4-methyltrityl)-D-α,γ-diaminobutyric acid, (N-γ-4-methyltrityl)-L-α,γ-diaminobutyric acid, (N-γ-allyloxycarbonyl)-L-α,γ-diaminobutyric acid, D-α,γ-diaminobutyric acid, 4,5-dehydro-L-leucine, cyclopentyl-D-Gly-OH, cyclopentyl-Gly-OH, D-allylglycine, D-homocyclohexylalanine, L-1-pyrenylalanine, L-2-aminocaproic acid, L-allylglycine, L-homocyclohexylalanine, and N-(2-hydroxy-4-methoxy-Bzl)-Gly-OH.

Exemplary amino acid analogs of arginine and lysine include, but are not limited to, citrulline, L-2-amino-3-guanidinopropionic acid, L-2-amino-3-ureidopropionic acid, L-citrulline, Lys(Me)₂-OH, Lys(N₃)—OH, Nδ-benzyloxycarbonyl-L-ornithine, Nω-nitro-D-arginine, Nω-nitro-L-arginine, α-methyl-ornithine, 2,6-diaminoheptanedioic acid, L-ornithine, (Nδ-1-(4,4-dimethyl-2,6-dioxo-cyclohex-1-ylidene)ethyl)-D-ornithine, (Nδ-1-(4,4-dimethyl-2,6-dioxo-cyclohex-1-ylidene)ethyl)-L-ornithine, (Nδ-4-methyltrityl)-D-ornithine, (Nδ-4-methyltrityl)-L-ornithine, D-ornithine, L-ornithine, Arg(Me)(Pbf)-OH, Arg(Me)₂-OH (asymmetrical), Arg(Me)₂-OH (symmetrical), Lys(ivDde)-OH, Lys(Me)₂-OH·HCl, Lys(Me₃)-OH chloride, Nω-nitro-D-arginine, and Nω-nitro-L-arginine.

Exemplary amino acid analogs of aspartic and glutamic acids include, but are not limited to, α-methyl-D-aspartic acid, α-methyl-glutamic acid, α-methyl-L-aspartic acid, γ-methylene-glutamic acid, (N-γ-ethyl)-L-glutamine, [N-α-(4-aminobenzoyl)]-L-glutamic acid, 2,6-diaminopimelic acid, L-α-aminosuberic acid, D-2-aminoadipic acid, D-α-aminosuberic acid, α-aminopimelic acid, iminodiacetic acid, L-2-aminoadipic acid, threo-β-methyl-aspartic acid, γ-carboxy-D-glutamic acid γ,γ-di-t-butyl ester, γ-carboxy-L-glutamic acid γ,γ-di-t-butyl ester, Glu(OAll)-OH, L-Asu(OtBu) OH, and pyroglutamic acid.

Exemplary amino acid analogs of cysteine and methionine include, but are not limited to, Cys(famesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillainine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Exemplary amino acid analogs of phenylalanine and tyrosine include, but are not limited to, (3-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2,4,5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Exemplary amino acid analogs of proline include 3,4-dehydro-proline, 4-fluoro-proline, cis-4-hydroxy-proline, thiazolidine-2-carboxylic acid, and trans-4-fluoro-proline.

Exemplary amino acid analogs of serine and threonine include 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Exemplary amino acid analogs of tryptophan include, but are not limited to, α-methyl-tryptophan, β-(3-benzothienyl)-D-alanine, β-(3-benzothienyl)-L-alanine, 1-methyl-tryptophan, 4-methyl-tryptophan, 5-benzyloxy-tryptophan, 5-bromo-tryptophan, 5-chloro-tryptophan, 5-fluoro-tryptophan, 5-hydroxy-tryptophan, 5-hydroxy-L-tryptophan, 5-methoxy-tryptophan, 5-methoxy-L-tryptophan, 5-methyl-tryptophan, 6-bromo-tryptophan, 6-chloro-D-tryptophan, 6-chloro-tryptophan, 6-fluoro-tryptophan, 6-methyl-tryptophan, 7-benzyloxy-tryptophan, 7-bromo-tryptophan, 7-methyl-tryptophan, D-1,2,3,4-tetrahydro-norharman-3-carboxylic acid, 6-methoxy-1,2,3,4-tetrahydronorharman-1-carboxylic acid, 7-azatryptophan, L-1,2,3,4-tetrahydro-norharman-3-carboxylic acid, 5-methoxy-2-methyl-tryptophan, and 6-chloro-L-tryptophan.

In some instances, an artificial nucleotide comprises, for example, modifications at one or more of ribose moiety, phosphate moiety, nucleoside moiety, or a combination thereof. In some instances, an artificial nucleotide comprises a nucleic acid with a modification at a 2′ hydroxyl group of the ribose moiety. In some cases, the modification is a 2′-O-methyl modification or a 2′-O-methoxyethyl (2′-O-MOE) modification. The 2′-O-methyl modification is added a methyl group to the 2′ hydroxyl group of the ribose moiety whereas the 2′O-methoxyethyl modification is added a methoxyethyl group to the 2′ hydroxyl group of the ribose moiety. In some cases, the 2′ hydroxyl group includes a 2′-O-aminopropyl sugar conformation which can involve an extended amine group comprising a propyl linker that binds the amine group to the 2′ oxygen. In some cases, the 2′ hydroxyl group includes a locked or bridged ribose conformation (e.g., locked nucleic acid or LNA) where the 4′ ribose position can also be involved. In this modification, the oxygen molecule bound at the 2′ carbon is linked to the 4′ carbon by a methylene group, thus forming a 2′-C,4′-C-oxy-methylene-linked bicyclic ribonucleotide monomer. In some cases, the 2′ hydroxyl group comprises ethylene nucleic acids (ENA) such as for example 2′-4′-ethylene-bridged nucleic acid, which locks the sugar conformation into a C3′-endo sugar puckering conformation. Iii additional cases, the 2′ hydroxyl group includes 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), or 2′-O—N-methylacetamido (2′-O-NMA).

In some embodiments, a nucleotide analogue further comprises a morpholino, a peptide nucleic acid (PNA), a methylphosphonate nucleotide, a thiolphosphonate nucleotide, 2′-fluoro N3-P5′-phosphoramidite, 1′,5′-anhydrohexitol nucleic acid (HNA), or a combination thereof.

In some embodiments, a ligand described herein comprises a small molecule ligand-electrophile compound.

### Kits/Article of Manufacture

Disclosed herein, in certain embodiments, are kits and articles of manufacture for use to generate JAK1 selective compounds of the present invention. In some embodiments, described herein is a kit for detecting protein ligand interaction. In some embodiments, such kit includes small molecule ligands described herein, small molecule fragments or libraries, compound probes described herein, and/or controls, and reagents suitable for carrying out one or more of the methods described herein. In some instances, the kit further comprises samples, such as a cell sample, and suitable solutions such as buffers or media. In some embodiments, the kit further comprises recombinant JAK1 protein for use in one or more of the methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastics.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include probes, test compounds, and one or more reagents for use in a method disclosed herein. Such kits optionally include an identifying description or label or instructions relating to its use in the methods described herein.

A kit typically includes labels listing contents and/or instructions for use, and package inserts with instructions for use. A set of instructions will also typically be included.

In some embodiments, a label is on or associated with the container. In some embodiment, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. The label also indicates directions for use of the contents, such as in the methods described herein.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, Fisher Scientific (Fisher Chemicals), and Acros Organics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Green and Wuts, Protective Groups in Organic Synthesis 3^{rd} Ed., (Wiley 1999) (all of which are incorporated by reference for such disclosure). General methods for the preparation of compounds as disclosed herein may be derived from reactions and the reactions may be modified by the use of appropriate reagents and conditions, for the introduction of the various moieties found in the formulae as provided herein. As a guide the following synthetic methods may be utilized.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure).

### Cells, Analytical Techniques, and Instrumentation

In certain embodiments, also described herein are methods for profiling a protein described above to determine a reactive or ligandable cysteine residue. In some instances, the methods comprising profiling a cell sample or a cell lysate sample. In some embodiments, the cell sample or cell lysate sample is obtained from cells of an animal. In some instances, the animal cell includes a cell from a marine invertebrate, fish, insects, amphibian, reptile, or mammal. In some instances, the mammalian cell is a primate, ape, equine, bovine, porcine, canine, feline, or rodent. In some instances, the mammal is a primate, ape, dog, cat, rabbit, ferret, or the like. In some cases, the rodent is a mouse, rat, hamster, gerbil, hamster, chinchilla, or guinea pig. In some embodiments, the bird cell is from a canary, parakeet or parrots. In some embodiments, the reptile cell is from a turtles, lizard or snake. In some cases, the fish cell is from a tropical fish. In some cases, the fish cell is from a zebrafish (e.g. Danino rerio). In some cases, the worm cell is from a nematode (e.g. C. elegans). In some cases, the amphibian cell is from a frog. In some embodiments, the arthropod cell is from a tarantula or hermit crab.

In some embodiments, the cell sample or cell lysate sample is obtained from a mammalian cell. In some instances, the mammalian cell is an epithelial cell, connective tissue cell, hormone secreting cell, a nerve cell, a skeletal muscle cell, a blood cell, or an immune system cell.

Exemplary mammalian cells include, but are not limited to, 293A cell line, 293FT cell line, 293F cells, 293 H cells, HEK 293 cells, CHO DG44 cells, CHO—S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO—S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, and PC12 cell line.

In some instances, the cell sample or cell lysate sample is obtained from cells of a tumor cell line. In some instances, the cell sample or cell lysate sample is obtained from cells of a solid tumor cell line. In some instances, the solid tumor cell line is a sarcoma cell line. In some instances, the solid tumor cell line is a carcinoma cell line. In some embodiments, the sarcoma cell line is obtained from a cell line of alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, telangiectatic osteosarcoma.

In some embodiments, the carcinoma cell line is obtained from a cell line of adenocarcinoma, squamous cell carcinoma, adenosquamous carcinoma, anaplastic carcinoma, large cell carcinoma, small cell carcinoma, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

In some instances, the cell sample or cell lysate sample is obtained from cells of a hematologic malignant cell line. In some instances, the hematologic malignant cell line is a T-cell cell line. In some instances, B-cell cell line. In some instances, the hematologic malignant cell line is obtained from a T-cell cell line of: peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some instances, the hematologic malignant cell line is obtained from a B-cell cell line of acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, the cell sample or cell lysate sample is obtained from a tumor cell line. Exemplary tumor cell line includes, but is not limited to, 600 MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

In some embodiments, the cell sample or cell lysate sample is from any tissue or fluid from an individual. Samples include, but are not limited to, tissue (e.g. connective tissue, muscle tissue, nervous tissue, or epithelial tissue), whole blood, dissociated bone marrow, bone marrow aspirate, pleural fluid, peritoneal fluid, central spinal fluid, abdominal fluid, pancreatic fluid, cerebrospinal fluid, brain fluid, ascites, pericardial fluid, urine, saliva, bronchial lavage, sweat, tears, ear flow, sputum, hydrocele fluid, semen, vaginal flow, milk, amniotic fluid, and secretions of respiratory, intestinal or genitourinary tract. In some embodiments, the cell sample or cell lysate sample is a tissue sample, such as a sample obtained from a biopsy or a tumor tissue sample. In some embodiments, the cell sample or cell lysate sample is a blood serum sample. In some embodiments, the cell sample or cell lysate sample is a blood cell sample containing one or more peripheral blood mononuclear cells (PBMCs). In some embodiments, the cell sample or cell lysate sample contains one or more circulating tumor cells (CTCs). In some embodiments, the cell sample or cell lysate sample contains one or more disseminated tumor cells (DTC, e.g., in a bone marrow aspirate sample).

In some embodiments, the cell sample or cell lysate sample is obtained from the individual by any suitable means of obtaining the sample using well-known and routine clinical methods. Procedures for obtaining tissue samples from an individual are well known. For example, procedures for drawing and processing tissue sample such as from a needle aspiration biopsy is well-known and is employed to obtain a sample for use in the methods provided. Typically, for collection of such a tissue sample, a thin hollow needle is inserted into a mass such as a tumor mass for sampling of cells that, after being stained, will be examined under a microscope.

### Sample Preparation and Analysis

In some embodiments, a sample solution comprises a cell sample, a cell lysate sample, or a sample comprising isolated proteins. In some instances, the sample solution comprises a solution such as a buffer (e.g. phosphate buffered saline) or a media. In some embodiments, the media is an isotopically labeled media. In some instances, the sample solution is a cell solution.

In some embodiments, the solution sample (e.g., cell sample, cell lysate sample, or comprising isolated proteins) is incubated with a compound of Formula (I), (II), or (III) for analysis of protein-probe interactions. In some instances, the solution sample (e.g., cell sample, cell lysate sample, or comprising isolated proteins) is further incubated in the presence of an additional compound probe prior to addition of the compound of Formula (I), (II), or (III). In other instances, the solution sample (e.g., cell sample, cell lysate sample, or comprising isolated proteins) is further incubated with a ligand, in which the ligand does not contain a photoreactive moiety and/or an alkyne group. In such instances, the solution sample is incubated with a probe and a ligand for competitive protein profiling analysis.

In some cases, the cell sample or the cell lysate sample is compared with a control. In some cases, a difference is observed between a set of probe protein interactions between the sample and the control. In some instances, the difference correlates to the interaction between the small molecule fragment and the proteins.

In some embodiments, one or more methods are utilized for labeling a solution sample (e.g. cell sample, cell lysate sample, or comprising isolated proteins) for analysis of probe protein interactions. In some instances, a method comprises labeling the sample (e.g. cell sample, cell lysate sample, or comprising isolated proteins) with an enriched media. In some cases, the sample (e.g. cell sample, cell lysate sample, or comprising isolated proteins) is labeled with isotope-labeled amino acids, such as ¹³C or ¹⁵N-labeled amino acids. In some cases, the labeled sample is further compared with a non-labeled sample to detect differences in probe protein interactions between the two samples. In some instances, this difference is a difference of a target protein and its interaction with a small molecule ligand in the labeled sample versus the non-labeled sample. In some instances, the difference is an increase, decrease or a lack of protein-probe interaction in the two samples. In some instances, the isotope-labeled method is termed SILAC, stable isotope labeling using amino acids in cell culture.

In some embodiments, a method comprises incubating a solution sample (e.g. cell sample, cell lysate sample, or comprising isolated proteins) with a labeling group (e.g., an isotopically labeled labeling group) to tag one or more proteins of interest for further analysis. In such cases, the labeling group comprises a biotin, a streptavidin, bead, resin, a solid support, or a combination thereof, and further comprises a linker that is optionally isotopically labeled. As described above, the linker can be about 5, 6, 7, 8, 9, 10, 11, 12, 13, 14, 15 or more residues in length and might further comprise a cleavage site, such as a protease cleavage site (e.g., TEV cleavage site). In some cases, the labeling group is a biotin-linker moiety, which is optionally isotopically labeled with ¹³C and ¹⁵N atoms at one or more amino acid residue positions within the linker. In some cases, the biotin-linker moiety is a isotopically-labeled TEV-tag as described in Weerapana, et al., “Quantitative reactivity profiling predicts functional cysteines in proteomes,” (Nature 468 (7325): 790-795).

In some embodiments, an isotopic reductive dimethylation (ReDi) method is utilized for processing a sample. In some cases, the ReDi labeling method involves reacting peptides with formaldehyde to form a Schiff base, which is then reduced by cyanoborohydride. This reaction dimethylates free amino groups on N-termini and lysine side chains and monomethylates N-terminal prolines. In some cases, the ReDi labeling method comprises methylating peptides from a first processed sample with a “light” label using reagents with hydrogen atoms in their natural isotopic distribution and peptides from a second processed sample with a “heavy” label using deuterated formaldehyde and cyanoborohydride. Subsequent proteomic analysis (e.g., mass spectrometry analysis) based on a relative peptide abundance between the heavy and light peptide version might be used for analysis of probe-protein interactions.

In some embodiments, isobaric tags for relative and absolute quantitation (iTRAQ) method is utilized for processing a sample. In some cases, the iTRAQ method is based on the covalent labeling of the N-terminus and side chain amines of peptides from a processed sample. In some cases, reagent such as 4-plex or 8-plex is used for labeling the peptides.

In some embodiments, the probe-protein complex is further conjugated to a chromophore, such as a fluorophore. In some instances, the probe-protein complex is separated and visualized utilizing an electrophoresis system, such as through a gel electrophoresis, or a capillary electrophoresis. Exemplary gel electrophoresis includes agarose-based gels, polyacrylamide based gels, or starch based gels. In some instances, the probe-protein is subjected to a native electrophoresis condition. In some instances, the probe-protein is subjected to a denaturing electrophoresis condition.

In some instances, the probe-protein after harvesting is further fragmentized to generate protein fragments. In some instances, fragmentation is generated through mechanical stress, pressure, or chemical means. In some instances, the protein from the probe-protein complexes is fragmented by a chemical means. In some embodiments, the chemical means is a protease. Exemplary proteases include, but are not limited to, serine proteases such as chymotrypsin A, penicillin G acylase precursor, dipeptidase E, DmpA aminopeptidase, subtilisin, prolyl oligopeptidase, D-Ala-D-Ala peptidase C, signal peptidase I, cytomegalovirus assemblin, Lon-A peptidase, peptidase Clp, Escherichia coli phage K1F endosialidase CIMCD self-cleaving protein, nucleoporin 145, lactoferrin, murein tetrapeptidase LD-carboxypeptidase, or rhomboid-1; threonine proteases such as ornithine acetyltransferase; cysteine proteases such as TEV protease, amidophosphoribosyltransferase precursor, gamma-glutamyl hydrolase (Rattus norvegicus), hedgehog protein, DmpA aminopeptidase, papain, bromelain, cathepsin K, calpain, caspase-1, separase, adenain, pyroglutamyl-peptidase I, sortase A, hepatitis C virus peptidase 2, sindbis virus-type nsP2 peptidase, dipeptidyl-peptidase VI, or DeSI-1 peptidase; aspartate proteases such as beta-secretase 1 (BACE1), beta-secretase 2 (BACE2), cathepsin D, cathepsin E, chymosin, napsin-A, nepenthesin, pepsin, plasmepsin, presenilin, or renin; glutamic acid proteases such as AfuGprA; and metalloproteases such as peptidase M48.

In some instances, the fragmentation is a random fragmentation. In some instances, the fragmentation generates specific lengths of protein fragments, or the shearing occurs at particular sequence of amino acid regions.

In some instances, the protein fragments are further analyzed by a proteomic method such as by liquid chromatography (LC) (e.g. high performance liquid chromatography), liquid chromatography-mass spectrometry (LC-MS), matrix-assisted laser desorption/ionization (MALDI-TOF), gas chromatography-mass spectrometry (GC-MS), capillary electrophoresis-mass spectrometry (CE-MS), or nuclear magnetic resonance imaging (NMR).

In some embodiments, the LC method is any suitable LC methods well known in the art, for separation of a sample into its individual parts. This separation occurs based on the interaction of the sample with the mobile and stationary phases. Since there are many stationary/mobile phase combinations that are employed when separating a mixture, there are several different types of chromatography that are classified based on the physical states of those phases. In some embodiments, the LC is further classified as normal-phase chromatography, reverse-phase chromatography, size-exclusion chromatography, ion-exchange chromatography, affinity chromatography, displacement chromatography, partition chromatography, flash chromatography, chiral chromatography, and aqueous normal-phase chromatography.

In some embodiments, the LC method is a high performance liquid chromatography (HPLC) method. In some embodiments, the HPLC method is further categorized as normal-phase chromatography, reverse-phase chromatography, size-exclusion chromatography, ion-exchange chromatography, affinity chromatography, displacement chromatography, partition chromatography, chiral chromatography, and aqueous normal-phase chromatography.

In some embodiments, the HPLC method of the present disclosure is performed by any standard techniques well known in the art. Exemplary HPLC methods include hydrophilic interaction liquid chromatography (HILIC), electrostatic repulsion-hydrophilic interaction liquid chromatography (ERLIC) and reverse phase liquid chromatography (RPLC).

In some embodiments, the LC is coupled to a mass spectroscopy as a LC-MS method. In some embodiments, the LC-MS method includes ultra-performance liquid chromatography-electrospray ionization quadrupole time-of-flight mass spectrometry (UPLC-ESI-QTOF-MS), ultra-performance liquid chromatography-electrospray ionization tandem mass spectrometry (UPLC-ESI-MS/MS), reverse phase liquid chromatography-mass spectrometry (RPLC-MS), hydrophilic interaction liquid chromatography-mass spectrometry (HILIC-MS), hydrophilic interaction liquid chromatography-triple quadrupole tandem mass spectrometry (HILIC-QQQ), electrostatic repulsion-hydrophilic interaction liquid chromatography-mass spectrometry (ERLIC-MS), liquid chromatography time-of-flight mass spectrometry (LC-QTOF-MS), liquid chromatography-tandem mass spectrometry (LC-MS/MS), multidimensional liquid chromatography coupled with tandem mass spectrometry (LC/LC-MS/MS). In some instances, the LC-MS method is LC/LC-MS/MS. In some embodiments, the LC-MS methods of the present disclosure are performed by standard techniques well known in the art.

In some embodiments, the GC is coupled to a mass spectroscopy as a GC-MS method. In some embodiments, the GC-MS method includes two-dimensional gas chromatography time-of-flight mass spectrometry (GC*GC-TOFMS), gas chromatography time-of-flight mass spectrometry (GC-QTOF-MS) and gas chromatography-tandem mass spectrometry (GC-MS/MS).

In some embodiments, CE is coupled to a mass spectroscopy as a CE-MS method. In some embodiments, the CE-MS method includes capillary electrophoresis-negative electrospray ionization-mass spectrometry (CE-ESI-MS), capillary electrophoresis-negative electrospray ionization-quadrupole time of flight-mass spectrometry (CE-ESI-QTOF-MS) and capillary electrophoresis-quadrupole time of flight-mass spectrometry (CE-QTOF-MS).

In some embodiments, the nuclear magnetic resonance (NMR) method is any suitable method well known in the art for the detection of one or more cysteine binding proteins or protein fragments disclosed herein. In some embodiments, the NMR method includes one dimensional (1D) NMR methods, two dimensional (2D) NMR methods, solid state NMR methods and NMR chromatography. Exemplary 1D NMR methods include ¹Hydrogen, ¹³Carbon, ¹⁵Nitrogen, ¹⁷Oxygen, ¹⁹Fluorine, ³¹Phosphorus, ³⁹Potassium, ²³Sodium, ³³Sulfur, ⁸⁷Strontium, ²⁷Aluminium, ⁴³Calcium, ³⁵Chlorine, ³⁷Chlorine, ⁶³Copper, ⁶⁵Copper, ⁵⁷Iron, ²⁵Magnesium, ¹⁹⁹Mercury or ⁶⁷Zinc NMR method, distortionless enhancement by polarization transfer (DEPT) method, attached proton test (APT) method and 1D-incredible natural abundance double quantum transition experiment (INADEQUATE) method. Exemplary 2D NMR methods include correlation spectroscopy (COSY), total correlation spectroscopy (TOCSY), 2D-INADEQUATE, 2D-adequate double quantum transfer experiment (ADEQUATE), nuclear overhauser effect spectroscopy (NOSEY), rotating-frame NOE spectroscopy (ROESY), heteronuclear multiple-quantum correlation spectroscopy (HMQC), heteronuclear single quantum coherence spectroscopy (HSQC), short range coupling and long range coupling methods. Exemplary solid state NMR method include solid state ¹³Carbon NMR, high resolution magic angle spinning (HR-MAS) and cross polarization magic angle spinning (CP-MAS) NMR methods. Exemplary NMR techniques include diffusion ordered spectroscopy (DOSY), DOSY-TOCSY and DOSY-HSQC.

In some embodiments, the protein fragments are analyzed by method as described in Weerapana et al., “Quantitative reactivity profiling predicts functional cysteines in proteomes,” Nature, 468:790-795 (2010).

In some embodiments, the results from the mass spectroscopy method are analyzed by an algorithm for protein identification. In some embodiments, the algorithm combines the results from the mass spectroscopy method with a protein sequence database for protein identification. In some embodiments, the algorithm comprises ProLuCID algorithm, Probity, Scaffold, SEQUEST, or Mascot.

In some embodiments, a value is assigned to each of the protein from the probe-protein complex. In some embodiments, the value assigned to each of the protein from the probe-protein complex is obtained from the mass spectroscopy analysis. In some instances, the value is the area-under-the curve from a plot of signal intensity as a function of mass-to-charge ratio. In some instances, the value correlates with the reactivity of a Lys residue within a protein.

In some instances, a ratio between a first value obtained from a first protein sample and a second value obtained from a second protein sample is calculated. In some instances, the ratio is greater than 2.5, 3, 3.5, 4, 4.5, 5, 6, 7, 8, 9, 10, 11, 12, 13, 14, 15, 16, 17, 18, 19, or 20. In some cases, the ratio is at most 20.

In some instances, the ratio is calculated based on averaged values. In some instances, the averaged value is an average of at least two, three, or four values of the protein from each cell solution, or that the protein is observed at least two, three, or four times in each cell solution and a value is assigned to each observed time. In some instances, the ratio further has a standard deviation of less than 12, 10, or 8.

In some instances, a value is not an averaged value. In some instances, the ratio is calculated based on value of a protein observed only once in a cell population. In some instances, the ratio is assigned with a value of 20.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₈ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

“Heteroalkylene” refers to an alkyl radical as described above where one or more carbon atoms of the alkyl is replaced with a O, N or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NI-2. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized π-electron system containing 4n+2 π electrons, where n is an integer. Aromatics can be optionally substituted. The term “aromatic” includes both aryl groups (e.g., phenyl, naphthalenyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

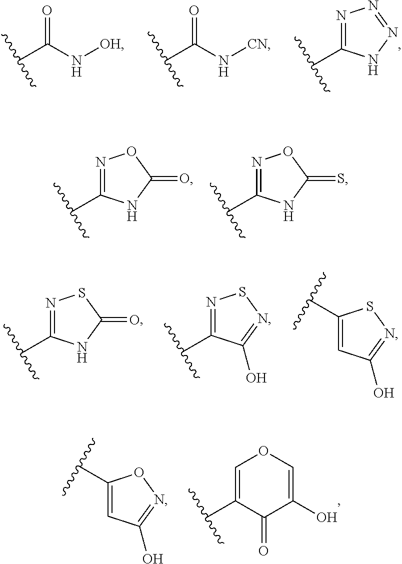

and the like.

“Cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cyclcoalkyl radicals include, for example, cyclopropyl, cyclobutyl, cyclopentyl, cyclohexyl, cycloheptyl, and cyclooctyl. In some embodiments, the monocyclic cyclcoalkyl is cyclopropyl, cyclobutyl, cyclopentyl or cyclohexyl. In some embodiments, the monocyclic cyclcoalkyl is cyclopentyl. Polycyclic radicals include, for example, adamantyl, norbornyl, decalinyl, and 3,4-dihydronaphthalen-1(2H)-one. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, a bicyclic heteroaryl is a C₆-C₉heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halogen, acyl, acyloxy, —CO₂H, —CO₂alkyl, nitro, and amino, including mono- and di-substituted amino groups (e.g. —NH₂, —NHR, —N(R)₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic, saturated or unsaturated carbon atoms, excluding aromatic carbon atoms) includes oxo (═O).

“Electrophile” is a chemical species that forms bonds with nucleophiles by accepting an electron pair. Such electrophiles are often involved in Michael addition reactions which involve the nucleophilic addition of a nucleophile to an α,β-unsaturated carbonyl compound containing an electron withdrawing group. It belongs to the larger class of conjugate additions and is widely used for the mild formation of C—C bonds. The term “Michael acceptor moiety” refers to a functional group that can participate in a Michael reaction, wherein a new covalent bond is formed between a portion of the Michael acceptor moiety and the donor moiety. The Michael acceptor moiety is an electrophile and the “donor moiety” is a nucleophile.

Non-limiting examples of compounds disclosed herein that are selective for JAK1 and with measurements of the micromolar quantities required for fifty percent target engagement of JAK1 and TYK2 are provided in Table 1.

TABLE 1
Exemplary compounds of the disclosure

| Compound | | JAK1 TE₅₀ in | TYK2 in vitro |
|---|---|---|---|
| No | Structure | vitro | TE₅₀ |
| 1 | 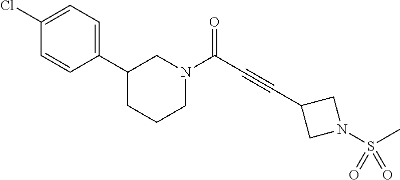 [SMILES: CS(=O)(=O)N1CC(C#CC(=O)N2CCCC(c3ccc(Cl)cc3)C2)C1] | 0.065 | 1.69 |
| 2 | 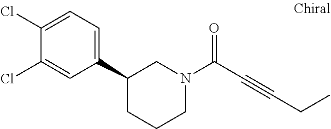 [SMILES: CCC#CC(=O)N1CCC[C@@H](c2ccc(Cl)c(Cl)c2)C1] | 0.07154 | 2.714 |
| 3 | 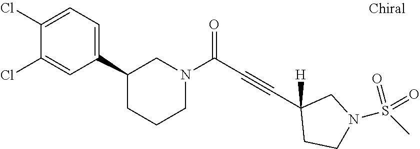 [SMILES: CS(=O)(=O)N1CC[C@H](C#CC(=O)N2CCC[C@@H](c3ccc(Cl)c(Cl)c3)C2)C1] | 0.007861 | 0.2049 |
| 4 | 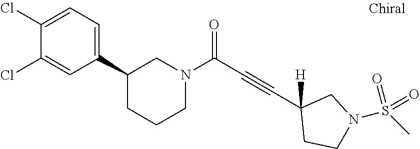 [SMILES: CS(=O)(=O)N1CC[C@H](C#CC(=O)N2CCC[C@@H](c3ccc(Cl)c(Cl)c3)C2)C1] | 0.01587 | 0.19 |
| 5 | 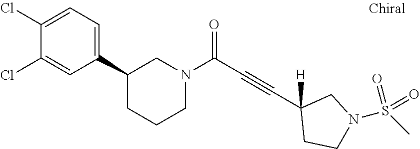 [SMILES: CS(=O)(=O)N1CC[C@H](C#CC(=O)N2CCC[C@@H](c3ccc(Cl)c(Cl)c3)C2)C1] | 0.07379 | 1.75 |
| 6 | 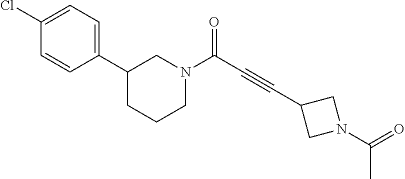 [SMILES: CC(=O)N1CC(C#CC(=O)N2CCCC(c3ccc(Cl)cc3)C2)C1] | 0.33 | 1.81 |
| 7 | 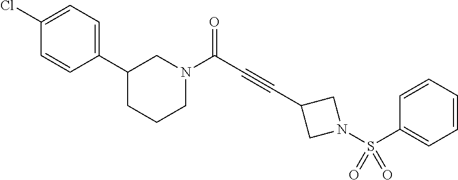 [SMILES: O=C(C#CC1CN(S(=O)(=O)c2ccccc2)C1)N1CCCC(c2ccc(Cl)cc2)C1] | 0.39 | 2.45 |
| 8 | 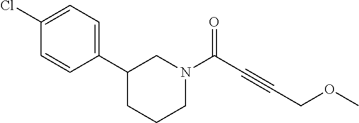 [SMILES: COCC#CC(=O)N1CCCC(c2ccc(Cl)cc2)C1] | 0.73 | 70.98 |
| 9 | 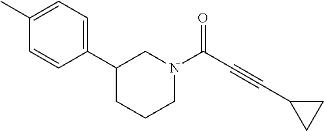 [SMILES: Cc1ccc(C2CCCN(C(=O)C#CC3CC3)C2)cc1] | 0.89 | |
| 10 | 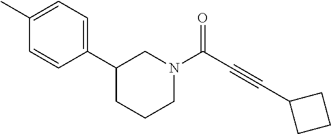 [SMILES: Cc1ccc(C2CCCN(C(=O)C#CC3CCC3)C2)cc1] | 0.62 | |
| 11 | 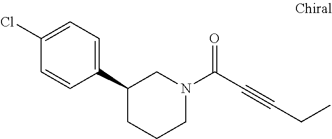 [SMILES: CCC#CC(=O)N1CCC[C@@H](c2ccc(Cl)cc2)C1] | 0.4876 | |
| 12 | 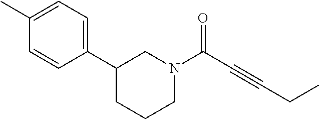 [SMILES: CCC#CC(=O)N1CCCC(c2ccc(C)cc2)C1] | 0.92 | |
| 13 | 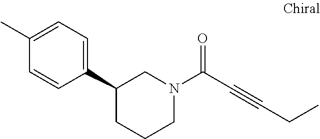 [SMILES: CCC#CC(=O)N1CCC[C@@H](c2ccc(C)cc2)C1] | 0.42 | |
| 14 | 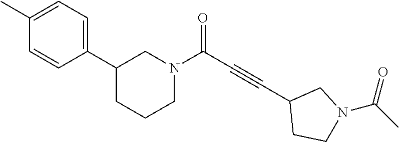 [SMILES: CC(=O)N1CCC(C#CC(=O)N2CCCC(c3ccc(C)cc3)C2)C1] | 0.47 | |
| 15 | 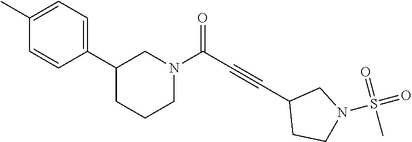 [SMILES: Cc1ccc(C2CCCN(C(=O)C#CC3CCN(S(C)(=O)=O)C3)C2)cc1] | 0.1051 | 1.15 |
| 16 | 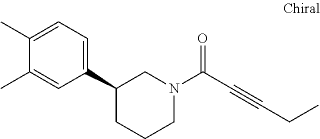 [SMILES: CCC#CC(=O)N1CCC[C@@H](c2ccc(C)c(C)c2)C1] | 0.6668 | 4.2 |
| 17 | 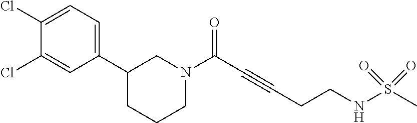 [SMILES: CS(=O)(=O)NCCC#CC(=O)N1CCCC(c2ccc(Cl)c(Cl)c2)C1] | 0.129 | 0.65 |
| 18 | 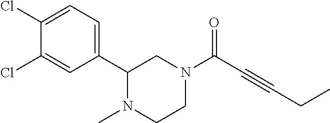 [SMILES: CCC#CC(=O)N1CCN(C)C(c2ccc(Cl)c(Cl)c2)C1] | 0.76 | |
| 19 | 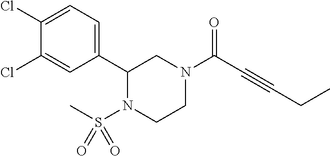 [SMILES: CCC#CC(=O)N1CCN(S(C)(=O)=O)C(c2ccc(Cl)c(Cl)c2)C1] | 0.7534 | 5.3 |
| 20 | 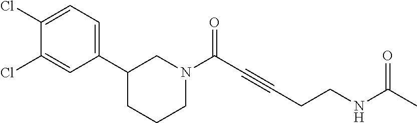 [SMILES: CC(=O)NCCC#CC(=O)N1CCCC(c2ccc(Cl)c(Cl)c2)C1] | 0.1543 | 4.2 |
| 21 | 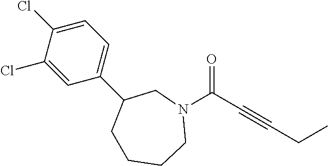 [SMILES: CCC#CC(=O)N1CCCCC(c2ccc(Cl)c(Cl)c2)C1] | 0.3243 | 11.24 |
| 22 | 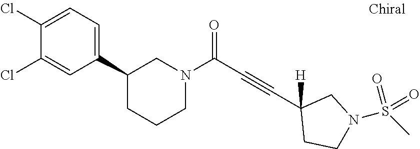 [SMILES: CS(=O)(=O)N1CC[C@H](C#CC(=O)N2CCC[C@@H](c3ccc(Cl)c(Cl)c3)C2)C1] | 0.08695 | 2.18 |
| 23 | 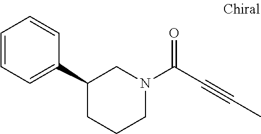 [SMILES: CC#CC(=O)N1CCC[C@@H](c2ccccc2)C1] | 3.74 | 86.08 |
| 24 | 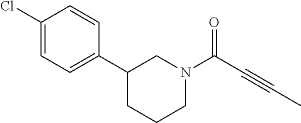 [SMILES: CC#CC(=O)N1CCCC(c2ccc(Cl)cc2)C1] | 4.584 | 85.61 |
| 25 | 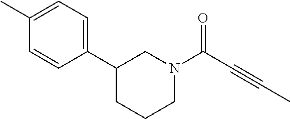 [SMILES: CC#CC(=O)N1CCCC(c2ccc(C)cc2)C1] | 6.96 | 179.4 |
| 26 | 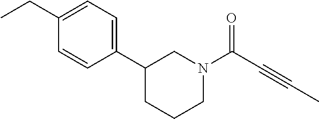 [SMILES: CC#CC(=O)N1CCCC(c2ccc(CC)cc2)C1] | 7.03 | 600 |
| 27 | 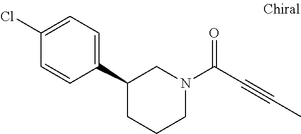 [SMILES: CC#CC(=O)N1CCC[C@@H](c2ccc(Cl)cc2)C1] | 2.926 | 43.68 |
| 28 | 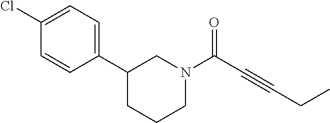 [SMILES: CCC#CC(=O)N1CCCC(c2ccc(Cl)cc2)C1] | 1.58 | 500 |
| 29 | 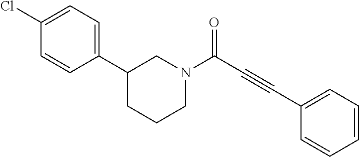 [SMILES: O=C(C#Cc1ccccc1)N1CCCC(c2ccc(Cl)cc2)C1] | 1.7 | 500 |
| 30 | 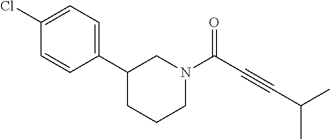 [SMILES: CC(C)C#CC(=O)N1CCCC(c2ccc(Cl)cc2)C1] | 1.16 | 14.2 |
| 31 | 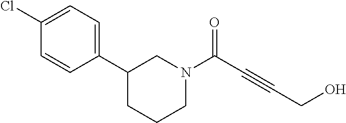 [SMILES: O=C(C#CCO)N1CCCC(c2ccc(Cl)cc2)C1] | 1.67 | 12.98 |
| 32 | 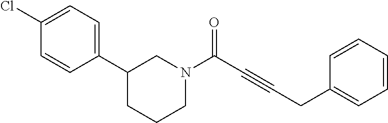 [SMILES: O=C(C#CCc1ccccc1)N1CCCC(c2ccc(Cl)cc2)C1] | 3.14 | 19.22 |
| 33 | 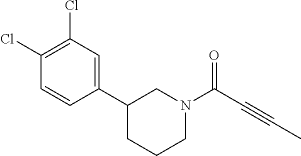 [SMILES: CC#CC(=O)N1CCCC(c2ccc(Cl)c(Cl)c2)C1] | 1.14 | 19.22 |
| 34 | 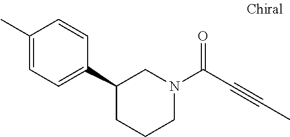 [SMILES: CC#CC(=O)N1CCC[C@@H](c2ccc(C)cc2)C1] | 2.9 | |
| 35 | 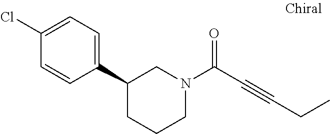 [SMILES: CCC#CC(=O)N1CCC[C@@H](c2ccc(Cl)cc2)C1] | 4.46 | |
| 36 | 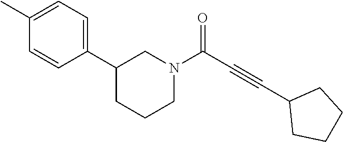 [SMILES: Cc1ccc(C2CCCN(C(=O)C#CC3CCCC3)C2)cc1] | 1.61 | |
| 37 | 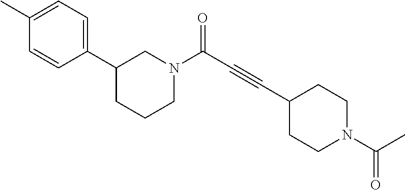 [SMILES: CC(=O)N1CCC(C#CC(=O)N2CCCC(c3ccc(C)cc3)C2)CC1] | 1.06 | |
| 38 | 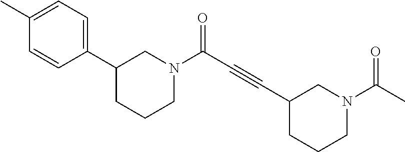 [SMILES: CC(=O)N1CCCC(C#CC(=O)N2CCCC(c3ccc(C)cc3)C2)C1] | 1.39 | |
| 39 | 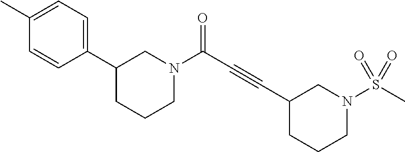 | 1.95 | |
| 40 | 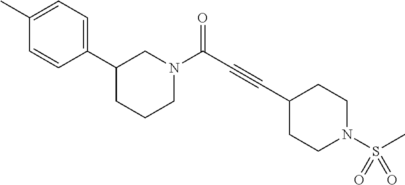 | 1.87 | |
| 41 | 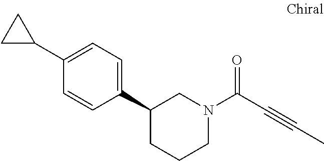 [SMILES: CC#CC(=O)N1CCC[C@@H](c2ccc(C3CC3)cc2)C1] | 7.78 | |
| 42 | 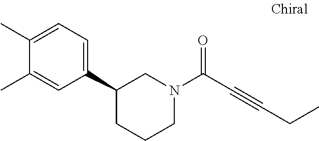 [SMILES: CCC#CC(=O)N1CCC[C@@H](c2ccc(C)c(C)c2)C1] | 4.82 | |
| 43 | 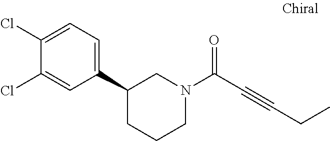 [SMILES: CCC#CC(=O)N1CCC[C@@H](c2ccc(Cl)c(Cl)c2)C1] | 3.49 | |
| 44 | 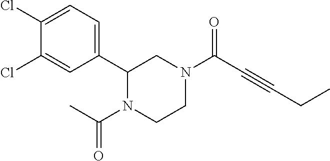 [SMILES: CCC#CC(=O)N1CCN(C(C)=O)C(c2ccc(Cl)c(Cl)c2)C1] | 4.56 | |
| 45 | 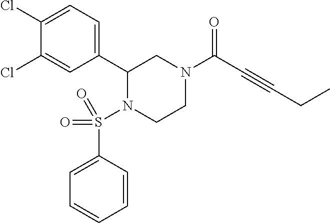 [SMILES: CCC#CC(=O)N1CCN(S(=O)(=O)c2ccccc2)C(c2ccc(Cl)c(Cl)c2)C1] | 4.92 | |
| 46 | 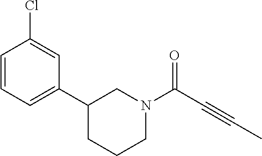 [SMILES: CC#CC(=O)N1CCCC(c2cccc(Cl)c2)C1] | 11.23 | 27.18 |
| 47 | 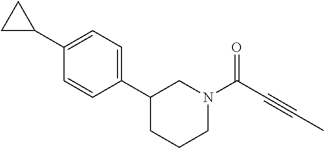 [SMILES: CC#CC(=O)N1CCCC(c2ccc(C3CC3)cc2)C1] | 19.94 | |
| 48 | 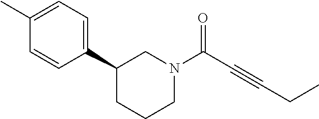 [SMILES: CCC#CC(=O)N1CCC[C@@H](c2ccc(C)cc2)C1] | 11.74 | |
| 49 | 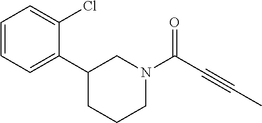 [SMILES: CC#CC(=O)N1CCCC(c2ccccc2Cl)C1] | 26.69 | 111.1 |
| 50 | 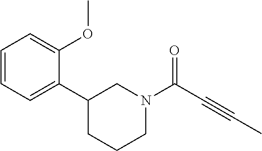 [SMILES: CC#CC(=O)N1CCCC(c2ccccc2OC)C1] | 120 | 500 |
| 51 | 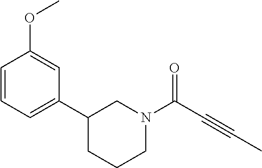 [SMILES: CC#CC(=O)N1CCCC(c2cccc(OC)c2)C1] | 150 | 330 |
| 52 | 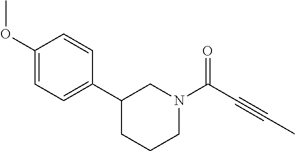 [SMILES: CC#CC(=O)N1CCCC(c2ccc(OC)cc2)C1] | 180 | 500 |
| 53 | 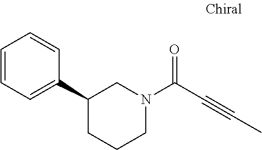 [SMILES: CC#CC(=O)N1CCC[C@@H](c2ccccc2)C1] | 141.4 | 500 |
| 54 | 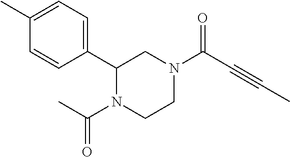 [SMILES: CC#CC(=O)N1CCN(C(C)=O)C(c2ccc(C)cc2)C1] | 242 | |
| 55 | 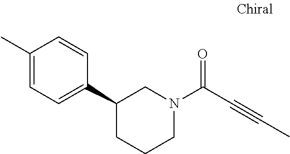 [SMILES: CC#CC(=O)N1CCC[C@@H](c2ccc(C)cc2)C1] | 125 | |
| 56 | 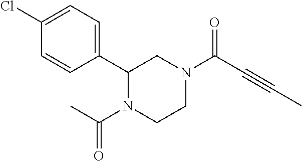 [SMILES: CC#CC(=O)N1CCN(C(C)=O)C(c2ccc(Cl)cc2)C1] | 200 | |
| 57 | 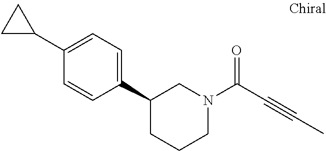 [SMILES: CC#CC(=O)N1CCC[C@@H](c2ccc(C3CC3)cc2)C1] | 50 | |
| 58 | 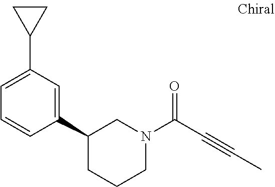 [SMILES: CC#CC(=O)N1CCC[C@@H](c2cccc(C3CC3)c2)C1] | 30 | |
| 59 | 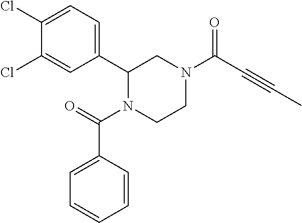 [SMILES: CC#CC(=O)N1CCN(C(=O)c2ccccc2)C(c2ccc(Cl)c(Cl)c2)C1] | 57.19 | |
| 60 | 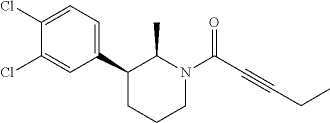 [SMILES: CCC#CC(=O)N1CCC[C@@H](c2ccc(Cl)c(Cl)c2)[C@H]1C] | 0.7 | 19.78 |
| 61 | 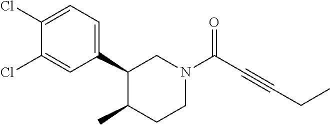 [SMILES: CCC#CC(=O)N1CC[C@@H](C)[C@@H](c2ccc(Cl)c(Cl)c2)C1] | 2.34 | 57.4 |
| 62 | 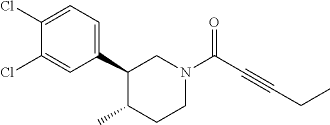 [SMILES: CCC#CC(=O)N1CC[C@H](C)[C@@H](c2ccc(Cl)c(Cl)c2)C1] | 6.74 | 21.04 |
| 63 | 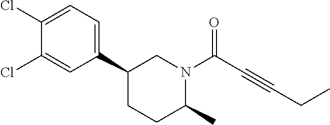 [SMILES: CCC#CC(=O)N1C[C@H](c2ccc(Cl)c(Cl)c2)CC[C@@H]1C] | 0.1984 | 5.727 |
| 64 | 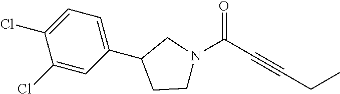 [SMILES: CCC#CC(=O)N1CCC(c2ccc(Cl)c(Cl)c2)C1] | 5.88 | 93.85 |
| 65 | 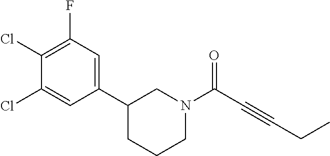 [SMILES: CCC#CC(=O)N1CCCC(c2cc(F)c(Cl)c(Cl)c2)C1] | 7.19 | 18.67 |
| 66 | 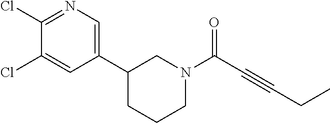 [SMILES: CCC#CC(=O)N1CCCC(c2cnc(Cl)c(Cl)c2)C1] | 3.95 | 17.87 |
| 67 | 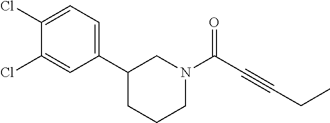 [SMILES: CCC#CC(=O)N1CCCC(c2ccc(Cl)c(Cl)c2)C1] | 0.1549 | 14.14 |
| 68 | 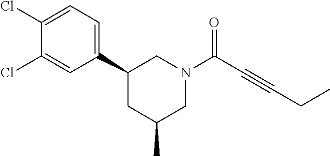 [SMILES: CCC#CC(=O)N1C[C@@H](C)C[C@@H](c2ccc(Cl)c(Cl)c2)C1] | 0.2592 | 7.19 |
| 69 | 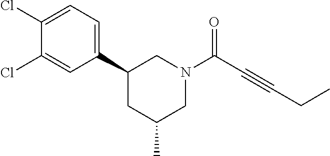 [SMILES: CCC#CC(=O)N1C[C@H](C)C[C@@H](c2ccc(Cl)c(Cl)c2)C1] | 0.3842 | 13.2 |
| 70 | 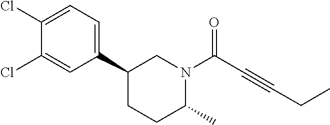 [SMILES: CCC#CC(=O)N1C[C@H](c2ccc(Cl)c(Cl)c2)CC[C@H]1C] | 10.88 | 500 |
| 71 | 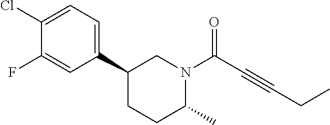 [SMILES: CCC#CC(=O)N1C[C@H](c2ccc(Cl)c(F)c2)CC[C@H]1C] | 0.6545 | 6.99 |
| 72 | 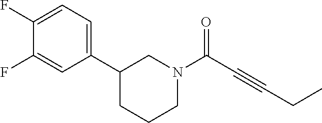 [SMILES: CCC#CC(=O)N1CCCC(c2ccc(F)c(F)c2)C1] | 2.13 | 29.68 |
| 73 | 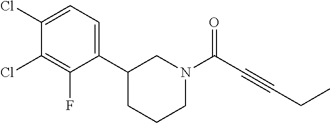 [SMILES: CCC#CC(=O)N1CCCC(c2ccc(Cl)c(Cl)c2F)C1] | 0.4345 | 16.32 |
| 74 | 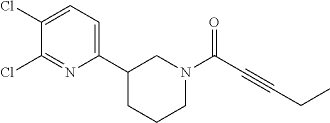 [SMILES: CCC#CC(=O)N1CCCC(c2ccc(Cl)c(Cl)n2)C1] | 0.1222 | 10 |
| 75 | 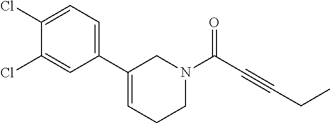 [SMILES: CCC#CC(=O)N1CCC=C(c2ccc(Cl)c(Cl)c2)C1] | 0.52 | 500 |
| 76 | 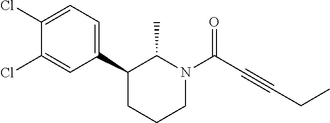 [SMILES: CCC#CC(=O)N1CCC[C@@H](c2ccc(Cl)c(Cl)c2)[C@@H]1C] | 12.28 | 20.96 |
| 77 | 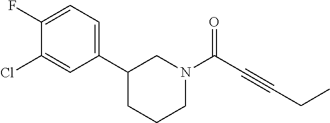 [SMILES: CCC#CC(=O)N1CCCC(c2ccc(F)c(Cl)c2)C1] | 0.55 | 4.16 |
| 78 | 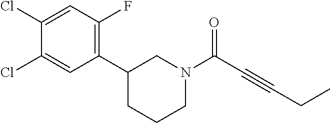 [SMILES: CCC#CC(=O)N1CCCC(c2cc(Cl)c(Cl)cc2F)C1] | 0.2828 | 4.43 |
| 79 | 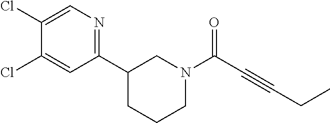 [SMILES: CCC#CC(=O)N1CCCC(c2cc(Cl)c(Cl)cn2)C1] | 0.92 | 10.53 |
| 80 | 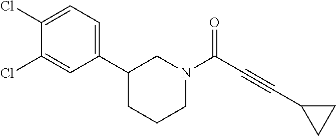 [SMILES: O=C(C#CC1CC1)N1CCCC(c2ccc(Cl)c(Cl)c2)C1] | 0.2511 | 3.05 |
| 81 | 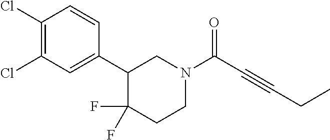 [SMILES: CCC#CC(=O)N1CCC(F)(F)C(c2ccc(Cl)c(Cl)c2)C1] | 0.03363 | 0.8175 |
| 82 | 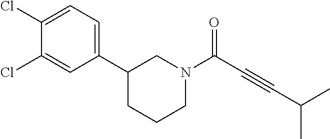 [SMILES: CC(C)C#CC(=O)N1CCCC(c2ccc(Cl)c(Cl)c2)C1] | 0.1123 | 4.83 |
| 83 | 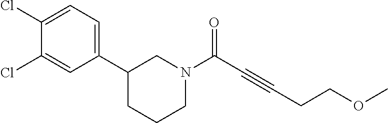 [SMILES: COCCC#CC(=O)N1CCCC(c2ccc(Cl)c(Cl)c2)C1] | 0.2993 | 4.07 |
| 84 | 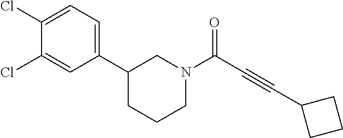 [SMILES: O=C(C#CC1CCC1)N1CCCC(c2ccc(Cl)c(Cl)c2)C1] | 0.12 | |
| 85 | 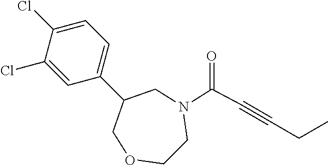 [SMILES: CCC#CC(=O)N1CCOCC(c2ccc(Cl)c(Cl)c2)C1] | 0.1949 | 6.91 |
| 86 | 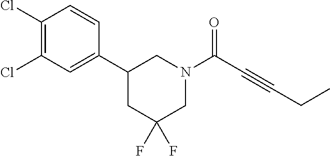 [SMILES: CCC#CC(=O)N1CC(c2ccc(Cl)c(Cl)c2)CC(F)(F)C1] | 0.1051 | 2.84 |
| 87 | 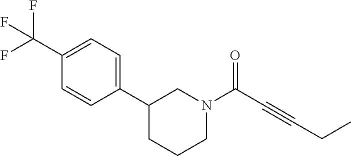 [SMILES: CCC#CC(=O)N1CCCC(c2ccc(C(F)(F)F)cc2)C1] | 0.55 | 7.71 |
| 88 | 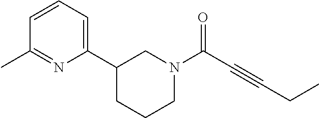 [SMILES: CCC#CC(=O)N1CCCC(c2cccc(C)n2)C1] | | 500 |
| 89 | 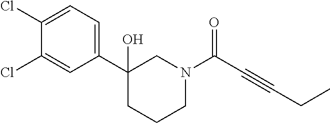 [SMILES: CCC#CC(=O)N1CCCC(O)(c2ccc(Cl)c(Cl)c2)C1] | 0.51 | |
| 90 | 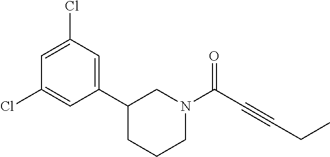 [SMILES: CCC#CC(=O)N1CCCC(c2cc(Cl)cc(Cl)c2)C1] | 1.61 | 88.84 |
| 91 | 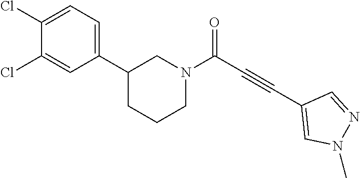 [SMILES: Cn1cc(C#CC(=O)N2CCCC(c3ccc(Cl)c(Cl)c3)C2)cn1] | 0.037 | |
| 92 | 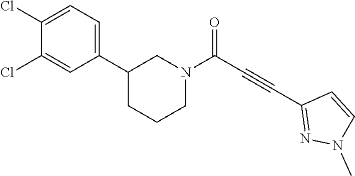 [SMILES: Cn1ccc(C#CC(=O)N2CCCC(c3ccc(Cl)c(Cl)c3)C2)n1] | 0.12 | |
| 93 | 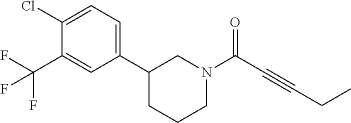 [SMILES: CCC#CC(=O)N1CCCC(c2ccc(Cl)c(C(F)(F)F)c2)C1] | 0.75 | |
| 94 | 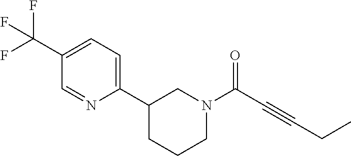 [SMILES: CCC#CC(=O)N1CCCC(c2ccc(C(F)(F)F)cn2)C1] | 0.64 | |
| 95 | 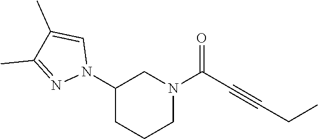 [SMILES: CCC#CC(=O)N1CCCC(n2cc(C)c(C)n2)C1] | 15 | 100 |
| 96 | 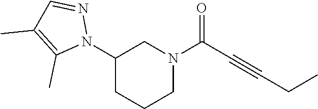 [SMILES: CCC#CC(=O)N1CCCC(n2ncc(C)c2C)C1] | 9.29 | |
| 97 | 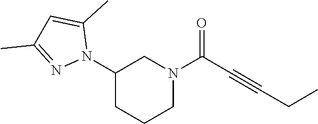 [SMILES: CCC#CC(=O)N1CCCC(n2nc(C)cc2C)C1] | | |
| 98 | 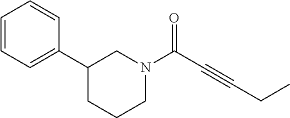 [SMILES: CCC#CC(=O)N1CCCC(c2ccccc2)C1] | | |
| 99 | 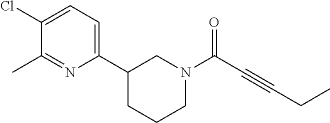 [SMILES: CCC#CC(=O)N1CCCC(c2ccc(Cl)c(C)n2)C1] | | |
| 100 | 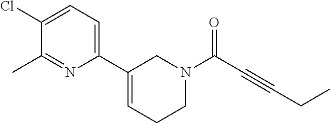 [SMILES: CCC#CC(=O)N1CCC=C(c2ccc(Cl)c(C)n2)C1] | | |
| 101 | 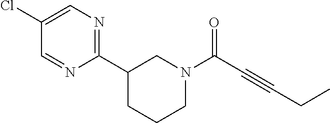 [SMILES: CCC#CC(=O)N1CCCC(c2ncc(Cl)cn2)C1] | 9.59 | |
| 102 | 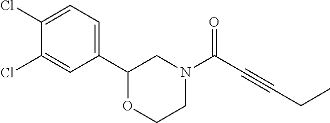 [SMILES: CCC#CC(=O)N1CCOC(c2ccc(Cl)c(Cl)c2)C1] | 0.075 | |
| 103 | 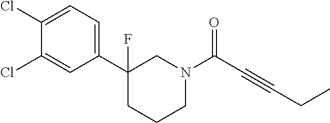 [SMILES: CCC#CC(=O)N1CCCC(F)(c2ccc(Cl)c(Cl)c2)C1] | 0.56 | |
| 104 | 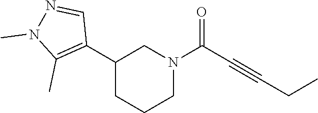 [SMILES: CCC#CC(=O)N1CCCC(c2cnn(C)c2C)C1] | | 100 |
| 105 | 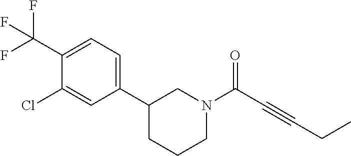 [SMILES: CCC#CC(=O)N1CCCC(c2ccc(C(F)(F)F)c(Cl)c2)C1] | | |
| 106 | 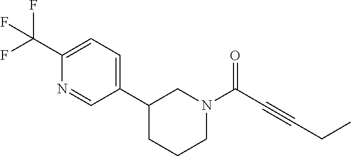 [SMILES: CCC#CC(=O)N1CCCC(c2ccc(C(F)(F)F)nc2)C1] | | |
| 107 | 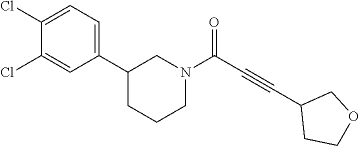 [SMILES: O=C(C#CC1CCOC1)N1CCCC(c2ccc(Cl)c(Cl)c2)C1] | | |
| 108 | 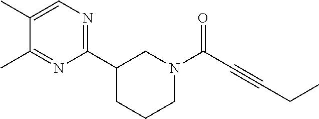 [SMILES: CCC#CC(=O)N1CCCC(c2ncc(C)c(C)n2)C1] | | |
| 109 | 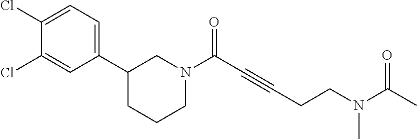 [SMILES: CC(=O)N(C)CCC#CC(=O)N1CCCC(c2ccc(Cl)c(Cl)c2)C1] | | |
| 110 | 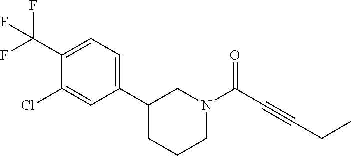 [SMILES: CCC#CC(=O)N1CCCC(c2ccc(C(F)(F)F)c(Cl)c2)C1] | | |
| 111 | 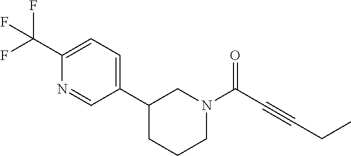 [SMILES: CCC#CC(=O)N1CCCC(c2ccc(C(F)(F)F)nc2)C1] | | |
| 112 | 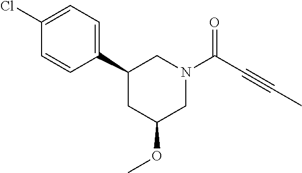 [SMILES: CC#CC(=O)N1C[C@@H](OC)C[C@@H](c2ccc(Cl)cc2)C1] | 6.7 | |
| 113 | 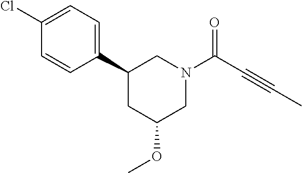 [SMILES: CC#CC(=O)N1C[C@H](OC)C[C@@H](c2ccc(Cl)cc2)C1] | 409 | |

Compounds in Table 2 are outside of Formula (I), although they have some structural similarities to compounds of the present invention, and the measurements of the micromolar quantities required for fifty percent target engagement of JAK1 and TYK2.

TABLE 2
Additional compounds of the disclosure.

| Compound | |
|---|---|
| No. | Structure |
| 114 | 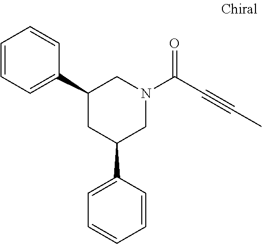 [SMILES: CC#CC(=O)N1C[C@H](c2ccccc2)C[C@H](c2ccccc2)C1] |
| 115 | 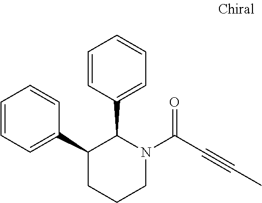 [SMILES: CC#CC(=O)N1CCC[C@@H](c2ccccc2)[C@H]1c1ccccc1] |
| 116 | 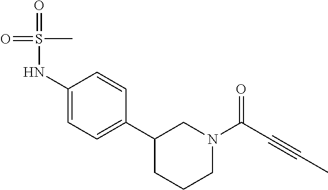 [SMILES: CC#CC(=O)N1CCCC(c2ccc(NS(C)(=O)=O)cc2)C1] |
| 117 | 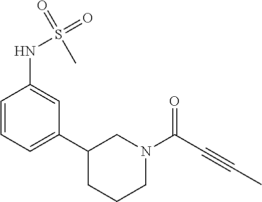 [SMILES: CC#CC(=O)N1CCCC(c2cccc(NS(C)(=O)=O)c2)C1] |
| 118 | 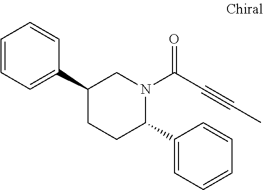 [SMILES: CC#CC(=O)N1C[C@H](c2ccccc2)CC[C@H]1c1ccccc1] |
| 119 | 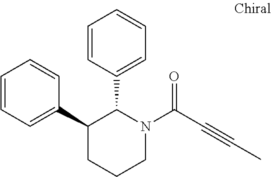 [SMILES: CC#CC(=O)N1CCC[C@@H](c2ccccc2)[C@@H]1c1ccccc1] |
| 120 | 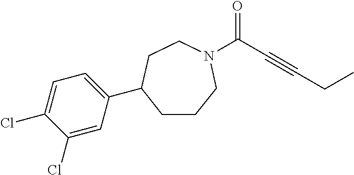 [SMILES: CCC#CC(=O)N1CCCC(c2ccc(Cl)c(Cl)c2)CC1] |

TABLE 3
Displays the amino acid sequences
disclosed herein.

| SEQ | | |
|---|---|---|
| ID | PRO- | |
| NO: | TEIN | PROTEIN AMINO ACID SEQUENCE |
| 1 | JAK1 | MQYLNIKEDC NAMAFCAKMR SSKKTEVNLE |
| | | APEPGVEVIF YLSDREPLRL GSGEYTAEEL |
| | | CIRAAQACRI SPLCHNLFAL YDENTKLWYA |
| | | PNRTITVDDK MSLRLHYRMR FYFTNWHGTN |
| | | DNEQSVWRHS PKKQKNGYEK KKIPDATPLL |
| | | DASSLEYLFA QGQYDLVKCL APIRDPKTEQ |
| | | DGHDIENECL GMAVLAISHY AMMKKMQLPE |
| | | LPKDISYKRY IPETLNKSIR QRNLLTRMRI |
| | | NNVFKDFLKE FNNKTICDSS VSTHDLKVKY |
| | | LATLETLTKH YGAEIFETSM LLISSENEMN |
| | | WFHSNDGGNV LYYEVMVTGN LGIQWRHKPN |
| | | VVSVEKEKNK LKRKKLENKH KKDEEKNKIR |
| | | EEWNNFSYFP EITHIVIKES VVSINKQDNK |
| | | KMELKLSSHE EALSFVSLVD GYFRLTADAH |
| | | HYLCTDVAPP LIVHNIQNGC HGPICTEYAI |
| | | NKLRQEGSEE GMYVLRWSCT DFDNILMTVT |
| | | CFEKSEQVQG AQKQFKNFQI EVQKGRYSLH |
| | | GSDRSFPSLG DLMSHLKKQI LRTDNISFML |
| | | KRCCQPKPRE ISNLLVATKK AQEWQPVYPM |
| | | SQLSFDRILK KDLVQGEHLG RGTRTHIYSG |
| | | TLMDYKDDEG TSEEKKIKVI LKVLDPSHRD |
| | | ISLAFFEAAS MMRQVSHKHI VYLYGVCVRD |
| | | VENIMVEEFV EGGPLDLFMH RKSDVLTTPW |
| | | KFKVAKQLAS ALSYLEDKDL VHGNVCTKNL |
| | | LLAREGIDSE CGPFIKLSDP GIPITVLSRQ |
| | | ECIERIPWIA PECVEDSKNL SVAADKWSFG |
| | | TTLWEICYNG EIPLKDKTLI EKERFYESRC |
| | | RPVTPSCKEL ADLMTRCMNY DPNQRPFFRA |
| | | IMRDINKLEE QNPDIVSEKK PATEVDPTHF |
| | | EKRFLKRIRD LGEGHFGKVE LCRYDPEGDN |
| | | TGEQVAVKSL KPESGGNHIA DLKKEIEILR |
| | | NLYHENIVKY KGICTEDGGN GIKLIMEFLP |
| | | SGSLKEYLPK NKNKINLKQQ LKYAVQICKG |
| | | MDYLGSRQYV HRDLAARNVL VESEHQVKIG |
| | | DFGLTKAIET DKEYYTVKDD RDSPVFWYAP |
| | | ECLMQSKFYI ASDVWSFGVT LHELLTYCDS |
| | | DSSPMALFLK MIGPTHGQMT VTRLVNTLKE |
| | | GKRLPCPPNC PDEVYQLMRK CWEFQPSNRT |
| | | SFQNLIEGFE ALLK |
| 2 | JAKI | MQYLNIKEDC NAMAFCAKMR SSKKTEVNLE |
| | (P733L) | APEPGVEVIF YLSDREPLRL GSGEYTAEEL |
| | | CIRAAQACRI SPLCHNLFAL YDENTKLWYA |
| | | PNRTITVDDK MSLRLHYRMR FYFTNWHGTN |
| | | DNEQSVWRHS PKKQKNGYEK KKIPDATPLL |
| | | DASSLEYLFA QGQYDLVKCL APIRDPKTEQ |
| | | DGHDIENECL GMAVLAISHY AMMKKMQLPE |
| | | LPKDISYKRY IPETLNKSIR QRNLLTRMRI |
| | | NNVFKDFLKE FNNKTICDSS VSTHDLKVKY |
| | | LATLETLTKH YGAEIFETSM LLISSENEMN |
| | | WFHSNDGGNV LYYEVMVTGN LGIQWRHKPN |
| | | VVSVEKEKNK LKRKKLENKH KKDEEKNKIR |
| | | EEWNNFSYFP EITHIVIKES VVSINKQDNK |
| | | KMELKLSSHE EALSFVSLVD GYFRLTADAH |
| | | HYLCTDVAPP LIVHNIQNGC HGPICTEYAI |
| | | NKLRQEGSEE GMYVLRWSCT DFDNILMTVT |
| | | CFEKSEQVQG AQKQFKNFQI EVQKGRYSLH |
| | | GSDRSFPSLG DLMSHLKKQI LRTDNISFML |
| | | KRCCQPKPRE ISNLLVATKK AQEWQPVYPM |
| | | SQLSFDRILK KDLVQGEHLG RGTRTHIYSG |
| | | TLMDYKDDEG TSEEKKIKVI LKVLDPSHRD |
| | | ISLAFFEAAS MMRQVSHKHI VYLYGVCVRD |
| | | VENIMVEEFV EGGPLDLFMH RKSDVLTTPW |
| | | KFKVAKQLAS ALSYLEDKDL VHGNVCTKNL |
| | | LLAREGIDSE CGLFIKLSDP GIPITVLSRQ |
| | | ECIERIPWIA PECVEDSKNL SVAADKWSFG |
| | | TTLWEICYNG EIPLKDKTLI EKERFYESRC |
| | | RPVTPSCKEL ADLMTRCMNY DPNQRPFFRA |
| | | IMRDINKLEE QNPDIVSEKK PATEVDPTHF |
| | | EKRFLKRIRD LGEGHFGKVE LCRYDPEGDN |
| | | TGEQVAVKSL KPESGGNHIA DLKKEIEILR |
| | | NLYHENIVKY KGICTEDGGN GIKLIMEFLP |
| | | SGSLKEYLPK NKNKINLKQQ LKYAVQICKG |
| | | MDYLGSRQYV HRDLAARNVL VESEHQVKIG |
| | | DFGLTKAIET DKEYYTVKDD RDSPVFWYAP |
| | | ECLMQSKFYI ASDVWSFGVT LHELLTYCDS |
| | | DSSPMALFLK MIGPTHGQMT VTRLVNTLKE |
| | | GKRLPCPPNC PDEVYQLMRK CWEFQPSNRT |
| | | SFQNLIEGFE ALLK |
| 3 | JAKI | MQYLNIKEDC NAMAFCAKMR SSKKTEVNLE |
| | (P832S) | APEPGVEVIF YLSDREPLRL GSGEYTAEEL |
| | | CIRAAQACRI SPLCHNLFAL YDENTKLWYA |
| | | PNRTITVDDK MSLRLHYRMR FYFTNWHGTN |
| | | DNEQSVWRHS PKKQKNGYEK KKIPDATPLL |
| | | DASSLEYLFA QGQYDLVKCL APIRDPKTEQ |
| | | DGHDIENECL GMAVLAISHY AMMKKMQLPE |
| | | LPKDISYKRY IPETLNKSIR QRNLLTRMRI |
| | | NNVFKDFLKE FNNKTICDSS VSTHDLKVKY |
| | | LATLETLTKH YGAEIFETSM LLISSENEMN |
| | | WFHSNDGGNV LYYEVMVTGN LGIQWRHKPN |
| | | VVSVEKEKNK LKRKKLENKH KKDEEKNKIR |
| | | EEWNNFSYFP EITHIVIKES VVSINKQDNK |
| | | KMELKLSSHE EALSFVSLVD GYFRLTADAH |
| | | HYLCTDVAPP LIVHNIQNGC HGPICTEYAI |
| | | NKLRQEGSEE GMYVLRWSCT DFDNILMTVT |
| | | CFEKSEQVQG AQKQFKNFQI EVQKGRYSLH |
| | | GSDRSFPSLG DLMSHLKKQI LRTDNISFML |
| | | KRCCQPKPRE ISNLLVATKK AQEWQPVYPM |
| | | SQLSFDRILK KDLVQGEHLG RGTRTHIYSG |
| | | TLMDYKDDEG TSEEKKIKVI LKVLDPSHRD |
| | | ISLAFFEAAS MMRQVSHKHI VYLYGVCVRD |
| | | VENIMVEEFV EGGPLDLFMH RKSDVLTTPW |
| | | KFKVAKQLAS ALSYLEDKDL VHGNVCTKNL |
| | | LLAREGIDSE CGPFIKLSDP GIPITVLSRQ |
| | | ECIERIPWIA PECVEDSKNL SVAADKWSFG |
| | | TTLWEICYNG EIPLKDKTLI EKERFYESRC |
| | | RPVTPSCKEL ADLMTRCMNY DSNQRPFFRA |
| | | IMRDINKLEE QNPDIVSEKK PATEVDPTHF |
| | | EKRFLKRIRD LGEGHFGKVE LCRYDPEGDN |
| | | TGEQVAVKSL KPESGGNHIA DLKKEIEILR |
| | | NLYHENIVKY KGICTEDGGN GIKLIMEFLP |
| | | SGSLKEYLPK NKNKINLKQQ LKYAVQICKG |
| | | MDYLGSRQYV HRDLAARNVL VESEHQVKIG |
| | | DFGLTKAIET DKEYYTVKDD RDSPVFWYAP |
| | | ECLMQSKFYI ASDVWSFGVT LHELLTYCDS |
| | | DSSPMALFLK MIGPTHGQMT VTRLVNTLKE |
| | | GKRLPCPPNC PDEVYQLMRK CWEFQPSNRT |
| | | SFQNLIEGFE ALLK |
| 4 | JAKI | MQYLNIKEDC NAMAFCAKMR SSKKTEVNLE |
| | (P733L, | APEPGVEVIF YLSDREPLRL GSGEYTAEEL |
| | P832S) | CIRAAQACRI SPLCHNLFAL YDENTKLWYA |
| | | PNRTITVDDK MSLRLHYRMR FYFTNWHGTN |
| | | DNEQSVWRHS PKKQKNGYEK KKIPDATPLL |
| | | DASSLEYLFA QGQYDLVKCL APIRDPKTEQ |
| | | DGHDIENECL GMAVLAISHY AMMKKMQLPE |
| | | LPKDISYKRY IPETLNKSIR QRNLLTRMRI |
| | | NNVFKDFLKE FNNKTICDSS VSTHDLKVKY |
| | | LATLETLTKH YGAEIFETSM LLISSENEMN |
| | | WFHSNDGGNV LYYEVMVTGN LGIQWRHKPN |
| | | VVSVEKEKNK LKRKKLENKH KKDEEKNKIR |
| | | EEWNNFSYFP EITHIVIKES VVSINKQDNK |
| | | KMELKLSSHE EALSFVSLVD GYFRLTADAH |
| | | HYLCTDVAPP LIVHNIQNGC HGPICTEYAI |
| | | NKLRQEGSEE GMYVLRWSCT DFDNILMTVT |
| | | CFEKSEQVQG AQKQFKNFQI EVQKGRYSLH |
| | | GSDRSFPSLG DLMSHLKKQI LRTDNISFML |
| | | KRCCQPKPRE ISNLLVATKK AQEWQPVYPM |
| | | SQLSFDRILK KDLVQGEHLG RGTRTHIYSG |
| | | TLMDYKDDEG TSEEKKIKVI LKVLDPSHRD |
| | | ISLAFFEAAS MMRQVSHKHI VYLYGVCVRD |
| | | VENIMVEEFV EGGPLDLFMH RKSDVLTTPW |
| | | KFKVAKQLAS ALSYLEDKDL VHGNVCTKNL |
| | | LLAREGIDSE CGLFIKLSDP GIPITVLSRQ |
| | | ECIERIPWIA PECVEDSKNL SVAADKWSFG |
| | | TTLWEICYNG EIPLKDKTLI EKERFYESRC |
| | | RPVTPSCKEL ADLMTRCMNY DSNQRPFFRA |
| | | IMRDINKLEE QNPDIVSEKK PATEVDPTHF |
| | | EKRFLKRIRD LGEGHFGKVE LCRYDPEGDN |
| | | TGEQVAVKSL KPESGGNHIA DLKKEIEILR |
| | | NLYHENIVKY KGICTEDGGN GIKLIMEFLP |
| | | SGSLKEYLPK NKNKINLKQQ LKYAVQICKG |
| | | MDYLGSRQYV HRDLAARNVL VESEHQVKIG |
| | | DFGLTKAIET DKEYYTVKDD RDSPVFWYAP |
| | | ECLMQSKFYI ASDVWSFGVT LHELLTYCDS |
| | | DSSPMALFLK MIGPTHGQMT VTRLVNTLKE |
| | | GKRLPCPPNC PDEVYQLMRK CWEFQPSNRT |
| | | SFQNLIEGFE ALLK |

The section headings used herein are for organizational purposes only and are not to be construed as limiting the subject matter described.

### EXAMPLES

These examples are provided for illustrative purposes only and not to limit the scope of the claims provided herein.

### Example 1: Synthesis of Compound 24

Described below is the representative experimental procedure for Scheme 1.

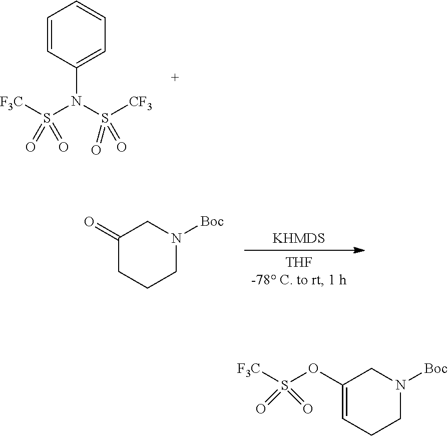

Step 1. To a solution prepared by diluting 1 M KHMDS in THF (63 mL, 63 mmol) with THF (100 mL) at −78° C. under N₂ (15 PSI) was added a solution of tert-butyl 3-oxopiperidine-1l-carboxylate (10.0 g, 50.2 mmol) in THF (100 mL) dropwise and the resulting mixture was stirred at −78° C. for 1.5 hours. A solution of N-phenylbis (trifluoromethanesulfonimide) (18.83 g, 52.7 mmol) in THF (100 mL) was added to the mixture dropwise at −78° C. and the mixture was stirred at −78° C. for 1 hour. The reaction mixture was allowed to warm to room temperature. After 1 hour, the mixture was quenched with a 100% NaOH solution (300 mL) and extracted with ethyl acetate (2×200 mL). The combined organic phases were washed with brine (500 mL), dried over Na₂SO₄, filtered, concentrated and purified by flash chromatography (0-10% v/v ethyl acetate in petroleum ether) to give tert-butyl 5-(trifluoromethylsulfonyloxy)-3,6-dihydro-2H-pyridine-1-carboxylate (13.00 g, 392.38 mmol) as a light yellow oil.

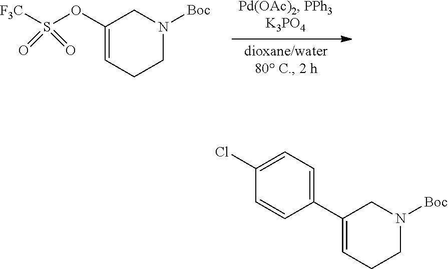

Step 2. A solution of tert-butyl 5-(trifluoromethylsulfonyloxy)-3,6-dihydro-2H-pyridine-1-carboxylate (1.00 g, 3.018 mmol), 4-chlorophenylboronic acid (0.47 g, 3.02 mmol), Pd(OAc)₂ (76.8 mg, 0.302 mmol), PPh₃ (158.3 mg, 0.604 mmol) and K₃PO₄ (0.83 g, 2 eq., 6.0 mmol) in a 10:1 mixture of 1,4-dioxane/water (33 mL, 0.092 M) was stirred at 80° C. for 2 hours. The mixture was concentrated and purified by flash chromatography (0-10% v/v ethyl acetate in petroleum ether) to give tert-butyl 5-(4-chlorophenyl)-3,6-dihydro-2H-pyridine-1-carboxylate (0.55 g, 1.872 mmol, 62.0% yield) as a yellow oil.

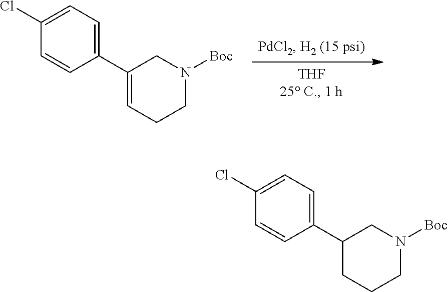 [SMILES: CC(C)(C)OC(=O)N1CCC=C(c2ccc(Cl)cc2)C1 ; "PdCl2, H2 (15 psi)" ; "THF25° C., 1 h" ; CC(C)(C)OC(=O)N1CCCC(c2ccc(Cl)cc2)C1]

Step 3. A mixture of tert-butyl 5-(4-chlorophenyl)-3,6-dihydro-2H-pyridine-1-carboxylate (300.0 mg, 1.021 mmol) and PdCl₂ (20.0 mg) in THF (10 mL, 0.102 M) was stirred under H₂ (15 PSI) at 25° C. for 1 hour. The reaction was concentrated and purified by acidic prep-HPLC (60-90% v/v solvent B) to give tert-butyl 3-(4-chlorophenyl)piperidine-1-carboxylate (130 mg, 0.434 mmol, 43% yield) as a white solid.

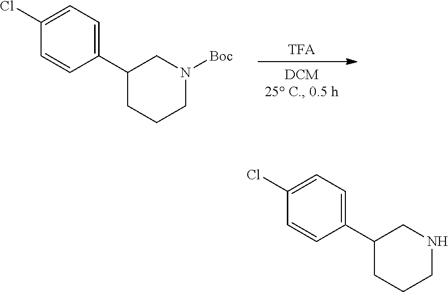 [SMILES: CC(C)(C)OC(=O)N1CCCC(c2ccc(Cl)cc2)C1 ; "DCM25° C., 0.5 h" ; "TFA" ; Clc1ccc(C2CCCNC2)cc1]

Step 4. A mixture of tert-butyl 3-(4-chlorophenyl) piperidine-1-carboxylate (130.0 mg, 0.44 mmol) in CH₂Cl₂ (2 mL) and TFA (2 mL) was stirred at 25° C. for 0.5 hours. The reaction was concentrated directly to give 3-(4-chlorophenyl)piperidine (80.0 mg, 0.409 mmol, 93.0% yield) as a yellow oil.

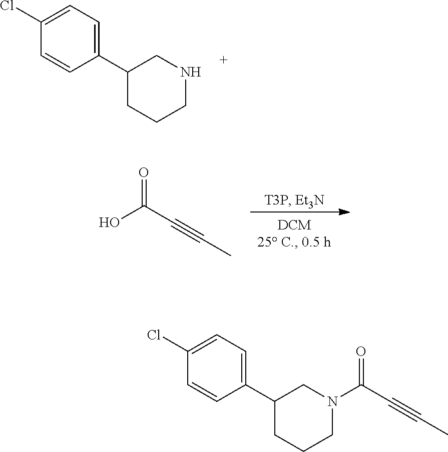

Step 5. To a solution of 3-(4-chlorophenyl)piperidine (80.0 mg, 0.409 mmol), Et₃N (82.8 mg, 0.818 mmol), and but-2-ynoic acid (68.7 mg, 0.818 mmol) in CH₂Cl₂ (4 mL, 0.102 M) was added T3P (260.0 mg, 0.818 mmol) at 25° C. and the reaction was stirred at 25° C. for 0.5 hour. The reaction was concentrated and purified by prep-TLC (1:1 petroleum ether:ethyl acetate) to give the title compound as a colorless oil. LC-MS m/z: 262.1 [M+1].

Following compounds were synthesized by Scheme 1 (Example 1) with appropriate reagents and purification methods including chiral resolution:

| Compound # | LC-MS m/z: |
|---|---|
| 8 | 282.1 |
| 9 | 268.2 |
| 10 | 282.2 |
| 11 | 276.1 |
| 12 | 256.2 |
| 13 | 256.2 |
| 21 | 324.1 |
| 23 | 228.1 |
| 25 | 242.1 |
| 26 | 256.1 |
| 27 | 262.0 |
| 28 | 276.1 |
| 29 | 324.1 |
| 30 | 290.2 |
| 33 | 296.1 |
| 34 | 242.2 |
| 35 | 276.1 |
| 46 | 262.1 |
| 48 | 256.1 |
| 49 | 262.0 |
| 50 | 258.2 |
| 51 | 258.2 |
| 52 | 258.1 |
| 53 | 228.1 |
| 98 | 242.3 |
| 79 | 311.0 |

### Example 2. Synthesis of Compound 2

Described below is the representative experimental procedure for Scheme 2.

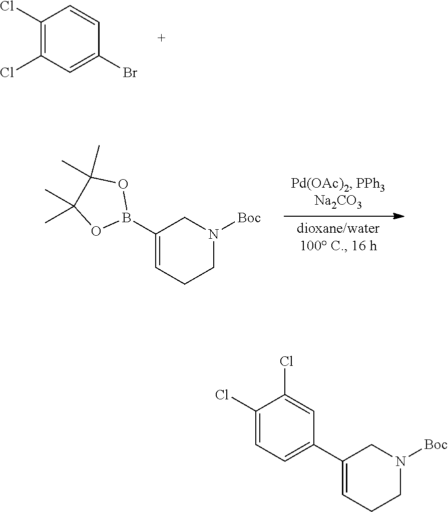

Step 1. To a solution of 1-bromo-3,4-dichlorobenzene (1.00 g, 4.427 mmol, 1 eq.), Na₂CO₃ (938.4 mg, 8.854 mmol, 2 eq.) and tert-butyl 5-(4,4,5,5-tetramethyl-1,3,2-dioxaborolan-2-yl)-3,6-dihydro-2H-pyridine-1-carboxylate (1.37 g, 4.427 mmol, 1 eq.) in dioxane/H₂O (5:1, 12 mL) was added PPh₃ (11.6 mg, 0.044 mmol, 0.01 eq.) and Pd(OAc)₂ (0.003 eq., 3.3 mg) at 20° C. and the mixture was stirred at 100° C. for 16 hours under N₂. The reaction was poured into water (15 mL) and extracted with ethyl acetate (3×5 mL). The organic layers were washed with brine (5 mL), dried over Na₂SO₄, filtered and concentrated under reduced pressure to give a residue. The residue was purified by flash chromatography (5-10% v/v ethyl acetate in petroleum ether) to give tert-butyl 5-(3,4-dichlorophenyl)-3,6-dihydro-2H-pyridine-1-carboxylate (1.20 g, 3.656 mmol, 82.6% yield) as a colorless oil.

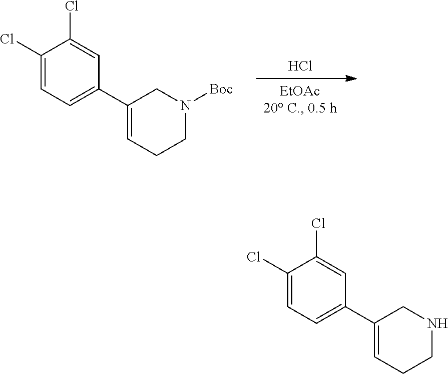

Step 2. A solution of tert-butyl 5-(3,4-dichlorophenyl)-3,6-dihydro-2H-pyridine-1-carboxylate (300.0 mg, 0.914 mmol) in HCl/ethyl acetate (2M, 3 mL) was stirred at 20° C. for 0.5 hour. The reaction was concentrated under reduced pressure to give crude 5-(3,4-dichlorophenyl)-1,2,3,6-tetrahydropyridine (200.0 mg, 0.877 mmol, 96% yield) as a white solid.

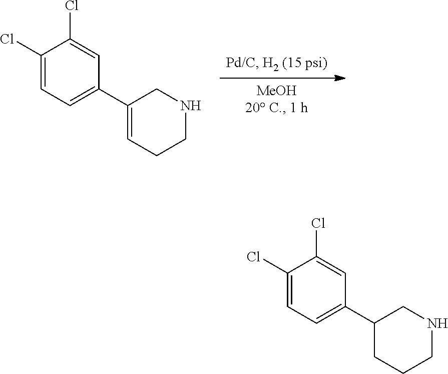 [SMILES: Clc1ccc(C2=CCCNC2)cc1Cl ; "MeOH20° C., 1 h" ; "Pd/C, H2 (15 psi)" ; Clc1ccc(C2CCCNC2)cc1Cl]

Step 3. To a solution of crude 5-(3,4-dichlorophenyl)-1,2,3,6-tetrahydropyridine (200.0 mg, 0.877 mmol) in MeOH (5 mL) was added Pd/C (5%, 10.0 mg) at 20° C. and the mixture was stirred under 15 PSI H₂ at the same temperature for 1 hour. The reaction was filtered over celite and concentrated under reduced pressure to give crude 3-(3,4-dichlorophenyl)piperidine (200.0 mg, 0.869 mmol, 99% yield) as a yellow solid.

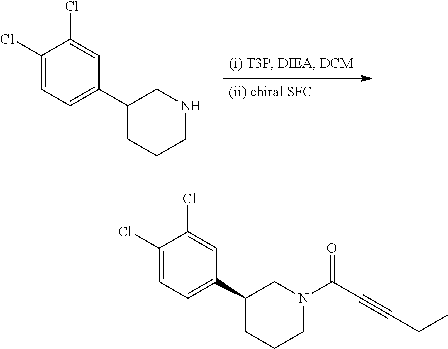 [SMILES: Clc1ccc(C2CCCNC2)cc1Cl ; "(ii) chiral SFC" ; "(i) T3P, DIEA, DCM" ; CCC#CC(=O)N1CCC[C@@H](c2ccc(Cl)c(Cl)c2)C1]

Step 4. To a solution of 3-(3,4-dichlorophenyl)piperidine (200.0 mg, 0.869 mmol), DIEA (337.0 mg, 2.607 mmol) and T3P (1.38 g, 2.173 mmol) in CH₂Cl₂ (3 mL) was added pent-2-ynoic acid (93.8 mg, 0.956 mmol) at 0° C. and the mixture was stirred at 20° C. for 0.5 hour. The reaction was poured into H₂O (8 mL) and extracted with CH₂Cl₂ (3×2 mL). The organic layers were washed with brine (2 mL), dried over Na₂SO₄, filtered and concentrated under reduced pressure to give a residue. The residue was purified by chiral SFC (OJ column, 10 to 40% isopropanol) to give the title compound as a white solid and single enantiomer (ee 99%). LC-MS m/z: 310.1 [M+1].

Following compounds were synthesized by Scheme 2 (Example 2) with appropriate reagents and purification methods including chiral resolution:

| Compound # | LC-MS m/z: [M + 1] |
|---|---|
| 16 | 270.2 |
| 41 | 268.2 |
| 43 | 310.1 |
| 47 | 268.1 |
| 87 | 310.2 |
| 88 | 257.2 |
| 90 | 310.1 |
| 93 | 344.2 |
| 94 | 311.2 |
| 99 | 291.0 |
| 100 | 289.0 |
| 101 | 278.1 |
| 104 | 260.3 |
| 110 | 344.2 |
| 111 | 311.2 |
| 64 | 296.0 |
| 65 | 328.0 |
| 66 | 311.0 |
| 67 | 310.0 |
| 71 | 293.9 |
| 72 | 278.1 |
| 73 | 328.1 |
| 74 | 308.0 |
| 77 | 294.0 |
| 78 | 328.1 |
| 80 | 322.0 |

Compound 108 is synthesized by Scheme 2 (Example 2) with appropriate reagents and purification methods.

### Example 3. Synthesis of Compound 17

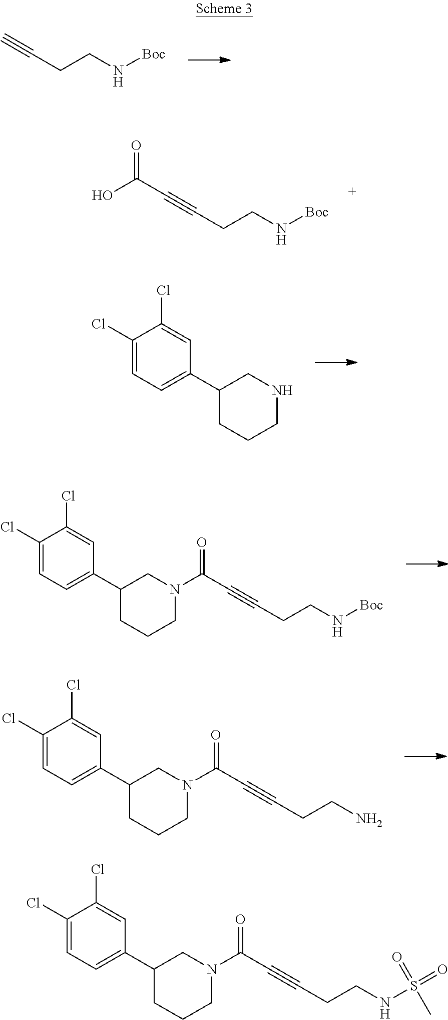 [SMILES: C#CCCNC(=O)OC(C)(C)C ; CC(C)(C)OC(=O)NCCC#CC(=O)O ; Clc1ccc(C2CCCNC2)cc1Cl ; CC(C)(C)OC(=O)NCCC#CC(=O)N1CCCC(c2ccc(Cl)c(Cl)c2)C1 ; NCCC#CC(=O)N1CCCC(c2ccc(Cl)c(Cl)c2)C1 ; CS(=O)(=O)NCCC#CC(=O)N1CCCC(c2ccc(Cl)c(Cl)c2)C1]

Step 1. To a solution of tert-butyl N-but-3-ynylcarbamate (200.0 mg, 1.182 mmol, 1 eq.) in THF (2 mL) was added dropwise n-BuLi (2.2 eq., 1.05 mL, 2.5 M) at −78° C. The mixture was stirred at −78° C. for 1 hour. To the mixture was then added dry ice (1.5 g) at −78° C. The mixture was stirred at −78° C. for 0.5 hours. The temperature of the reaction mixture was gradually raised to room temperature. The reaction mixture was quenched with water (10 mL), and then 2M hydrochloric acid was added to adjust to pH=3 and the mixture was extracted with ethyl acetate (3×3 mL). The combined organic layers were dried over Na₂SO₄, filtered and concentrated under reduced pressure to give 5-(tert-butoxycarbonylamino)pent-2-ynoic acid (250 mg, 1.172 mmol, 99% yield) as yellow oil.

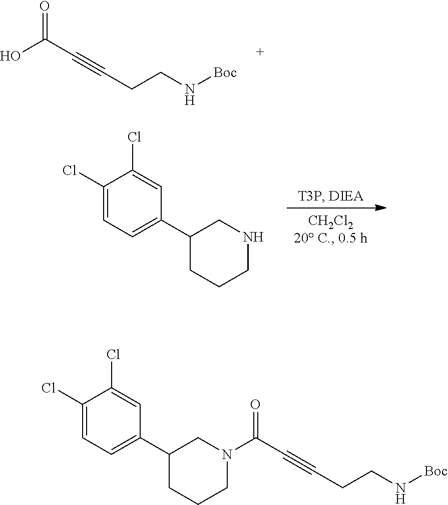 [SMILES: CC(C)(C)OC(=O)NCCC#CC(=O)O ; Clc1ccc(C2CCCNC2)cc1Cl ; "CH2Cl220° C., 0.5 h" ; "T3P, DIEA" ; CC(C)(C)OC(=O)NCCC#CC(=O)N1CCCC(c2ccc(Cl)c(Cl)c2)C1]

Step 2. To a solution of 3-(3,4-dichlorophenyl)piperidine (269.8 mg, 1.172 mmol), DIEA (454.6 mg, 3.517 mmol) and T3P (1.87 g, 2.931 mmol) in CH₂Cl₂ (4 mL) was added 5-(tert-butoxycarbonylamino)pent-2-ynoic acid (250 mg, 1.172 mmol) at 0° C. and the mixture was warmed to 20° C. and stirred for 0.5 hour. The reaction was poured into H₂O (30 mL) and extracted with CH₂Cl₂ (3×8 mL). The organic layers were washed with brine (10 mL), dried over Na₂SO₄, filtered and concentrated under reduced pressure to give a residue. The residue was purified by flash chromatography (10-40% v/v ethyl acetate in petroleum ether) to give tert-butyl N-[5-[3-(3,4-dichlorophenyl)-1-piperidyl]-5-oxo-pert-3-ynyl]carbamate (240 mg, 0.564 mmol, 48% yield) as a yellow oil.

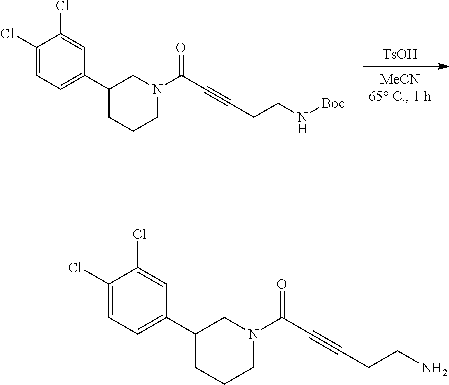 [SMILES: CC(C)(C)OC(=O)NCCC#CC(=O)N1CCCC(c2ccc(Cl)c(Cl)c2)C1 ; "TsOH" ; "MeCN65° C., 1 h" ; NCCC#CC(=O)N1CCCC(c2ccc(Cl)c(Cl)c2)C1]

Step 3. To a solution of tert-butyl N-[5-[3-(3,4-dichlorophenyl)-1-piperidyl]-5-oxo-pert-3-ynyl]carbamate (200 mg, 0.470 mmol, 1 eq.) in MeCN (2 mL) was added TsOH (81.0 mg, 0.470 mmol, 1 eq.) at 20° C. and the mixture was stirred at 65° C. for 1 hour. The reaction was concentrated under reduced pressure to give crude 5-amino-1-[3-(3,4-dichlorophenyl)-1-piperidyl]pent-2-yn-1-one tosylate salt (150 mg) as white solid.

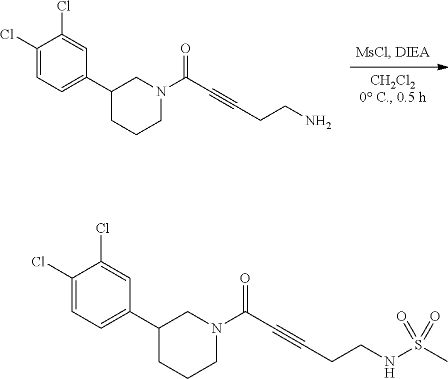 [SMILES: NCCC#CC(=O)N1CCCC(c2ccc(Cl)c(Cl)c2)C1 ; "CH2Cl20° C., 0.5 h" ; "MsCl, DIEA" ; CS(=O)(=O)NCCC#CC(=O)N1CCCC(c2ccc(Cl)c(Cl)c2)C1]

Step 4. To a solution of 5-amino-1-[3-(3,4-dichlorophenyl)-1-piperidyl]pent-2-yn-1-one (90 mg, 0.277 mmol) in CH₂Cl₂ (1 mL) was added DIEA (35.8 mg, 0.277 mmol) and MsCl (31.7 mg, 0.277 mmol) at 0° C. and the reaction was stirred at 0° C. for 0.5 hour. The reaction was poured into water (7 mL) and extracted with CH₂Cl₂ (3×2 mL). The combined organic layers were washed with brine (2 mL), dried over Na₂SO₄, filtered and concentrated under reduced pressure to give a residue. The residue was purified by acidic prep-HPLC (column: Nano-micro Kromasil C18 100*30 mm 8 μm) to give the title compound (56.0 mg, 0.139 mmol, 50% yield over two steps) as a yellow oil. LC-MS m/z: 403.1 [M+1].

Following compounds were synthesized by Scheme 3 (Example 3) with appropriate reagents and purification methods including chiral resolution:

| Compound # | LC-MS m/z: [M + 1] |
|---|---|
| 1 | 381.1 |
| 3 | 429.0 |
| 4 | 429.0 |
| 5 | 429.0 |
| 6 | 345.1 |
| 7 | 443.1 |
| 14 | 339.2 |
| 15 | 375.2 |
| 20 | 367.1 |
| 22 | 429.1 |
| 31 | 278.1 |
| 32 | 338.1 |
| 36 | 296.2 |
| 37 | 353.2 |
| 38 | 353.2 |
| 39 | 389.2 |
| 40 | 389.2 |
| 91 | 362.2 |
| 92 | 362.1 |
| 109) | 381.0 |

Compound 107 is synthesized by Scheme 3 (Example 3) with appropriate reagents and purification methods

### Example 4. Synthesis of Compound 18

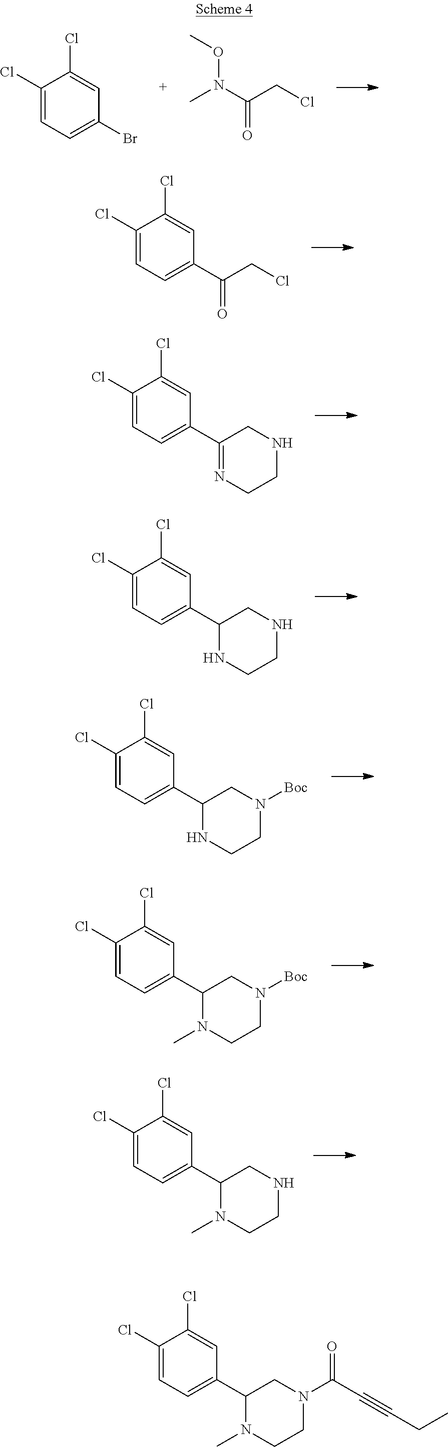

Described below is the representative experimental procedure for Scheme 4.

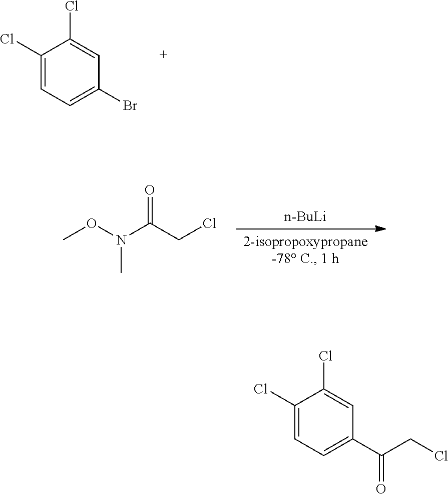

Step 1. To a solution of 1-bromo-3,4-dichlorobenzene (5 g, 22.13 mmol, 1 eq.) in 2-isopropoxypropane (50 mL) was added n-BuLi (2.5 M, 8 mL, 20 mmol, 0.9 eq.) at −78° C. and the mixture was stirred at the same temperature for 0.5 hour. Then 2-chloro-N-methoxy-N-methyl-acetamide (3.65 g, 26.56 mmol, 1.2 eq.) was added the mixture and the mixture was stirred at −78° C. for 1 hour. The reaction was poured into water (100 mL) and extracted with EtOAc (80 mL×3). The organic layers were washed with brine (150 mL), dried over Na₂SO₄, filtered and concentrated under reduced pressure to give a residue. The residue was purified by column chromatography (SiO₂, petroleum ether/ethyl acetate=100/1-20/1) to give 2-chloro-1-(3,4-dichlorophenyl)ethanone (4.0 g, 17.9 mmol, 81% yield) as a yellow oil.

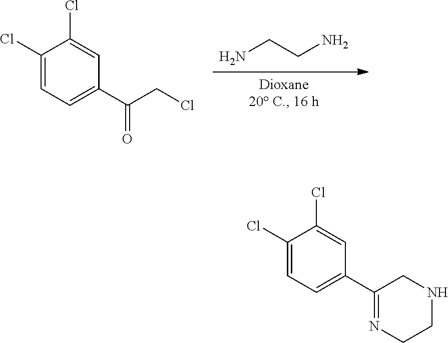

Step 2. To a solution of 2-chloro-1-(3,4-dichlorophenyl)ethanone (1 g, 4.47 mmol, 1 eq.) and 4A molecular sieve (1 g) in 1,4-dioxane (15 mL, 0.298 M) was added ethane-1,2-diamine (1.6 g, 26.62 mmol, 1.6 eq.) at 20° C. and the reaction was stirred at the same temperature for 16 hours. The reaction mixture was concentrated under reduced pressure to give crude 5-(3,4-dichlorophenyl)-1,2,3,6-tetrahydropyrazine (1.0 g) as a yellow oil.

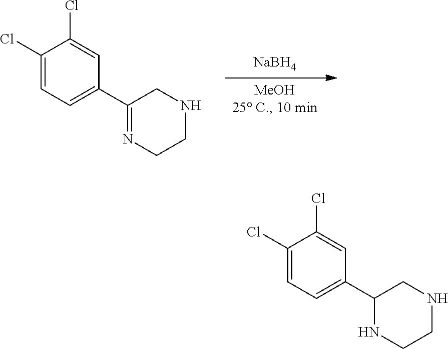 [SMILES: Clc1ccc(C2=NCCNC2)cc1Cl ; "MeOH25° C., 10 min" ; "NaBH4" ; Clc1ccc(C2CNCCN2)cc1Cl]

Step 3. To a solution of 5-(3,4-dichlorophenyl)-1,2,3,6-tetrahydropyrazine (1 g, 4.36 mmol, 1 eq.) in methanol (30 mL, 0.146 M) was added NaBH₄ (182 mg, 4.8 mmol, 1.1 eq.) at 0° C. The reaction mixture was stirred at 25° C. for 10 minutes. To the mixture was added 2N HCl solution until pH=4. The mixture was concentrated under reduced pressure to give a residue. The residue was dissolved with water (20 mL), washed with MTBE (20 mL×2). The aqueous phase was adjusted with saturated NaHCO₃ to pH=8. The aqueous phase was then extracted with EtOAc (20 mL×3). The organic phase was dried over Na₂SO₄, filtered and concentrated to give crude 2-(3,4-dichlorophenyl)piperazine (550 mg) as a yellow solid.

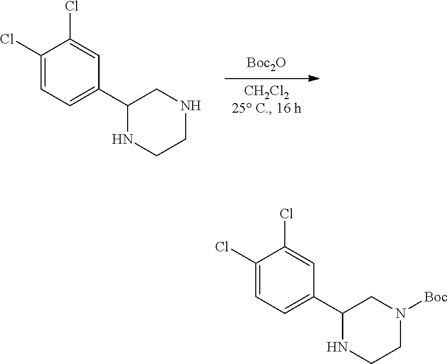 [SMILES: "Boc2O" ; Clc1ccc(C2CNCCN2)cc1Cl ; "CH2Cl225° C., 16 h" ; CC(C)(C)OC(=O)N1CCNC(c2ccc(Cl)c(Cl)c2)C1]

Step 4. To a solution of 2-(3,4-dichlorophenyl)piperazine (450 mg, 1.947 mmol, 1 eq.) in DCM (10 mL, 0.195 M) was added di-tert-butyl dicarbonate (467.44 mg, 2.142 mmol, 1.1 eq.) and the mixture was stirred at 25° C. for 16 hours. The mixture was concentrated and purified by flash column chromatography (silica gel, PE:EtOAc=1:1 to 0:1) to give tert-butyl 3-(3,4-dichlorophenyl)piperazine-1-carboxylate (400 mg, 1.21 mmol, 62% yield) as a yellow solid.

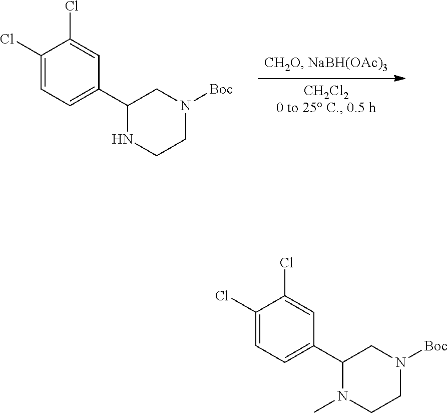

Step 5. To a solution of tert-butyl 3-(3,4-dichlorophenyl)piperazine-1-carboxylate (120 mg, 0.362 mmol, 1 eq.) in DCM (10 mL, 0.0362 M) was added formaldehyde (147 mg, 1.811 mmol, 37%, 5 eq.), NaBH(OAc)₃ (384 mg, 1.811 mmol, 6 eq.) and one drop of acetic acid at 0° C. and the reaction was stirred at 25° C. for 0.5 hour. The reaction was quenched with water (10 mL) and extracted with DCM (10 mL). The combined organic layers were washed with brine (10 mL), dried over Na₂SO₄, filtered and concentrated under reduced pressure to give crude tert-butyl 3-(3,4-dichlorophenyl)-4-methyl-piperazine-1-carboxylate (120 mg) as a yellow solid.

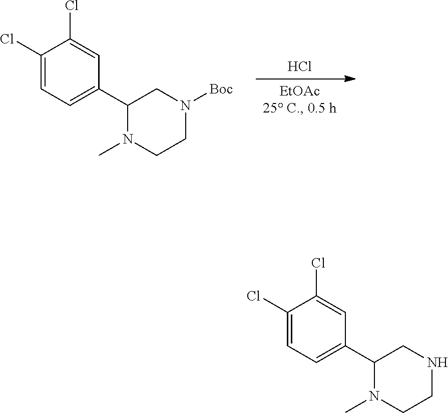

Step 6. A solution of tert-butyl 3-(3,4-dichlorophenyl)-4-methyl-piperazine-1-carboxylate (120 mg, 0.348 mmol) in 4N HCl/EtOAc (10 mL) was stirred at 25° C. for 0.5 hour. The mixture was concentrated under reduced pressure to give crude 2-(3,4-dichlorophenyl)-1-methyl-piperazine (85 mg) as a white solid.

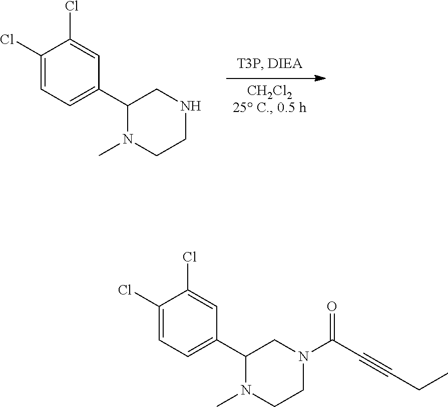 [SMILES: CN1CCNCC1c1ccc(Cl)c(Cl)c1 ; "CH2Cl225° C., 0.5 h" ; "T3P, DIEA" ; CCC#CC(=O)N1CCN(C)C(c2ccc(Cl)c(Cl)c2)C1]

Step 7. To a mixture of 2-(3,4-dichlorophenyl)-1-methyl-piperazine (85 mg, 0.347 mmol, 1 eq.), pent-2-ynoic acid (68.0 mg, 0.694 mmol, 2 eq.) and N,N-diisopropylethylamine (224.05 mg, 1.734 mmol, 5 eq.) in DCM (10 mL, 0.0347 M) was added 2,4,6-tripropyl-1,3,5,2,4,6-trioxatriphosphorinane-2,4,6-trioxide (441.3 mg, 0.694 mmol, 2 eq.) dropwise at 25° C. and the mixture was stirred at 25° C. for 0.5 hours. The reaction mixture was concentrated under reduced pressure and the residue was purified by prep-TLC (PE:EtOAc=1:1) to give the title compound (59.7 mg, 0.183 mmol, 53% yield) as a colorless oil. LC-MS m/z: 325.1 [M+1].

Following compounds were synthesized by Scheme 4 (Example 4) with appropriate reagents and purification methods:

| Compound # | LC-MS m/z: [M + 1] |
|---|---|
| 19 | 389.0 |
| 44 | 353.1 |
| 45 | 451.0 |

### Example 5. Synthesis of Compound 97

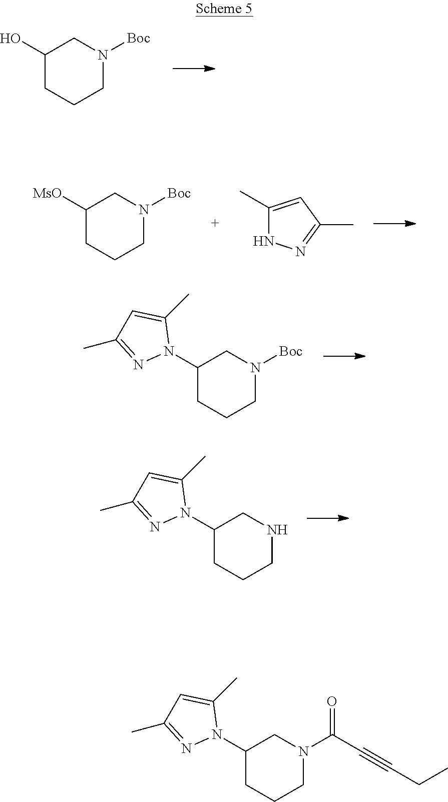

Described below is the representative experimental procedure for Scheme 5.

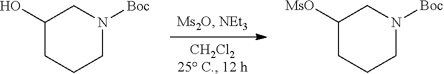

Step 1. To a solution of tert-butyl 3-hydroxypiperidine-1-carboxylate (2 g, 9.937 mmol, I eq.) in DCM (20 mL, 0.497 M) was added triethylamine (2.0 g, 19.874 mmol, 2 eq.) and methanesulfonic anhydride (2.6 g, 14.905 mmol, 1.5 eq.) at 0° C. The mixture was stirred for 12 hours at 25° C. The reaction mixture was quenched with water (20 mL) and extracted with DCM (20 mL×2). The combined organic layer was washed with brine (10 mL) and dried over Na₂SO₄, filtered and concentrated under reduced pressure to give a residue. The residue was purified by flash silica gel chromatography (ISCO®; 12 g SepaFlash® Silica Flash Column, Eluent of 0˜25% Ethyl acetate/petroleum ether gradient at 60 mL/min) to give tert-butyl 3-methylsulfonyloxypiperidine-1-carboxylate (2.50 g, 8.95 mmol, 90% yield) as a yellow oil.

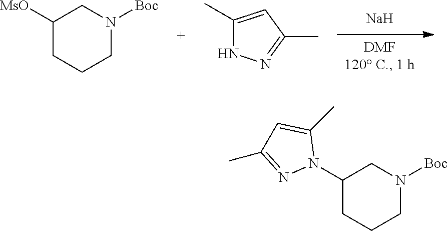

Step 2. To a solution of 3,5-dimethylpyrazole (103 mg, 1.074 mmol, 1 eq.) in DMF (5 mL, 0.215 M) was added NaH (100 mg, 1.8 eq.). The mixture was stirred at 0° C. for 0.5 hour, then tert-butyl 3-methylsulfonyloxypiperidine-1-carboxylate (300 mg, 1.074 mmol, 1 eq.) was added. The mixture was heated at 120° C. for 1 hour. The reaction mixture was diluted with aqueous saturated NH₄Cl (10 mL) and extracted with EtOAc (10 mL×3). The combined organic layers were washed with brine (5 mL), dried over Na₂SO₄, filtered and concentrated under reduced pressure to give a residue. The residue was purified by flash silica gel chromatography (ISCO®; 4 g SepaFlash® Silica Flash Column, Eluent of 0-100% Ethyl acetate/Petroleum ether gradient at 100 mL/min) to give tert-butyl 3-(3,5-dimethylpyrazol-1-yl)piperidine-1-carboxylate (60 mg, 0.215 mmol, 20% yield) as a colorless oil.

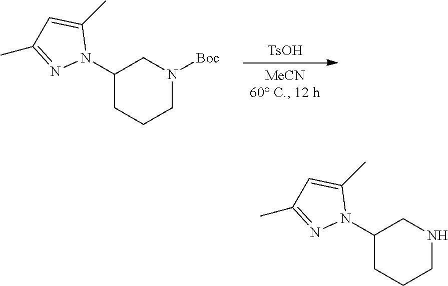 [SMILES: Cc1cc(C)n(C2CCCN(C(=O)OC(C)(C)C)C2)n1 ; "TsOH" ; "MeCN60° C., 12 h" ; Cc1cc(C)n(C2CCCNC2)n1]

Step 3. To a solution of tert-butyl 3-(3,5-dimethylpyrazol-1-yl)piperidine-1-carboxylate (60 mg, 0.215 mmol, 1 eq.) in MeCN (2 mL, 0.107 M) was added TsOH (2 eq, 50 mg). The mixture was stirred at 60° C. for 12 hours. The mixture was concentrated under reduced pressure to give crude 3-(3,5-dimethylpyrazol-1-yl)piperidine (30 mg, 0.167 mmol, 78% yield) as a white solid.

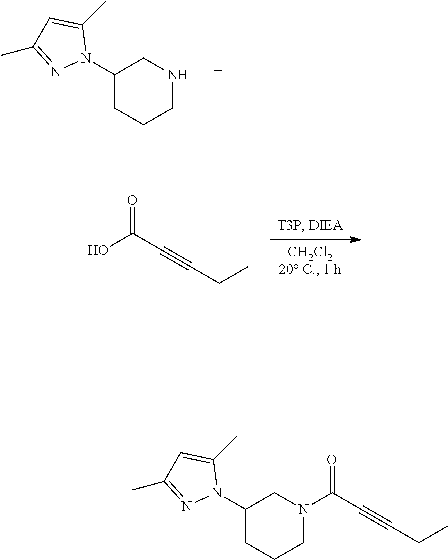

Step 4. To a solution of 3-(3,5-dimethylpyrazol-1-yl)piperidine (30 mg, 0.167 mmol, 1 eq.) in DCM (3 mL, 0.0558 M) was added 2-pentynoic acid (16.42 mg, 0.167 mmol, 1 eq.) and N,N-diisopropylethylamine (216.3 mg, 1.674 mmol, 10 eq), 2,4,6-tripropyl-1,3,5,2,4,6-trioxatriphosphorinane-2,4,6-trioxide (133.1 mg, 0.418 mmol, 2.5 eq.). The mixture was stirred at 20° C. for 1 hour. The reaction mixture was diluted with H₂O (10 mL) and extracted with DCM (10 mL×3). The combined organic layers were washed with brine (5 mL), dried over Na₂SO₄, filtered and concentrated under reduced pressure to give a residue. The residue was purified by prep-HPLC (Waters Xbridge Prep OBD C18 (150*40 mm*10 μm), water(NH₄HCO₃)-ACN, 8-55% B) to afford the title compound (15.6 mg, 0.0602 mmol, 36% yield) as a pale yellow oil. LC-MS m/z: 260.2 [M+1].

Following compounds were synthesized by Scheme 5 (Example 5) with appropriate reagents and purification methods:

| Compound # | LC-MS m/z: [M + 1] |
|---|---|
| 95 | 260.1 |
| 96 | 260.1 |

### Example 6. Synthesis of Compound 60

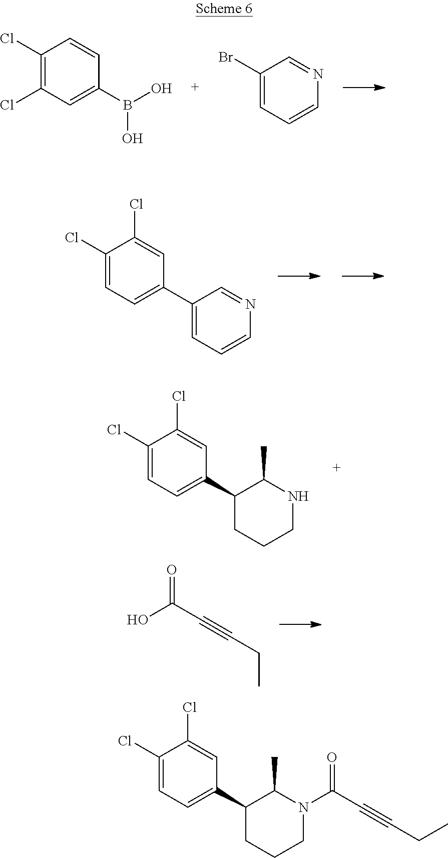

Described below is the representative experimental procedure for Scheme 6.

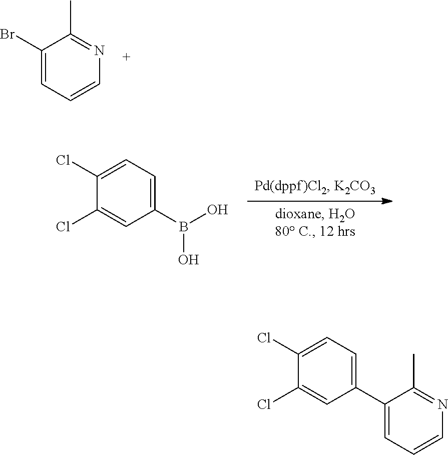

Step 1. To a solution of (3,4-dichlorophenyl)boronic acid (0.7 g, 3.67 mmol, 1 eq.) in dioxane (15 mL) and water (3 mL) (0.2 M) was added 3-bromo-2-methylpyridine (0.631 g, 3.67 mmol, 1 eq.), K₂CO₃ (1.27 g, 9.17 mmol, 2.5 eq.) and Pd(dppf)Cl₂ (268.4 mg, 0.367 mmol, 0.1 eq.). The mixture was stirred for 12 hours at 80° C. The reaction mixture was diluted with water (15 mL) and extracted with EtOAc (10 mL×3). The combined organic layers were washed with brine (10 mL) and dried over Na₂SO₄, filtered and concentrated under reduced pressure to give a residue. The residue was purified by flash silica gel chromatography (ISCO®; 12 g SepaFlash® Silica Flash Column, Eluent of 1˜99% Ethyl acetate/petroleum ether gradient at 60 mL/min) to give 3-(3,4-dichlorophenyl)-2-methylpyridine (0.8 g, 3.36 mmol, 92% yield) as a white solid.

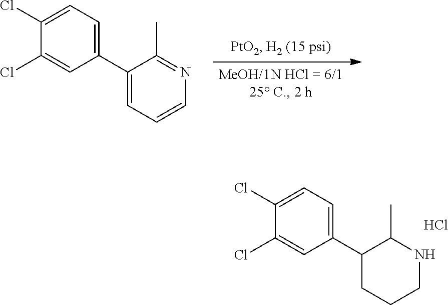

Step 2. To a solution of 3-(3,4-dichlorophenyl)-2-methylpyridine (400 mg, 1.68 mmol, 1 eq.) in MeOH (6 mL, 0.28 M) was added PtO₂ (30 mg, 0.13 mmol 0.08 eq.) and HCl (1 M, 1 mL, 0.6 eq.). The mixture was stirred at 25° C. for 12 hours under H₂ (15 psi). The reaction mixture was filtered and concentrated under reduced pressure to provide 3-(3,4-dichlorophenyl)-2-methylpiperidine hydrochloride (400 mg, 1.64 mmol, 97.5% yield).

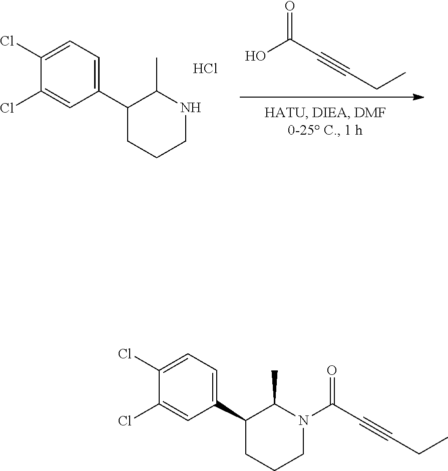

Step 3. To a solution of 3-(3,4-dichlorophenyl)-2-methylpiperidine hydrochloride (150 mg, 0.54 mmol, 1 eq.) in DMF (1.5 mL, 0.18 M) was added 2-pentynoic acid (90.4 mg, 0.922 mmol, 1.7 eq.) and N,N-diisopropylethylamine (238.2 mg, 1.84 mmol, 3.4 eq), (1-[Bis(dimethylamino)methylene]-1H-1,2,3-triazolo[4,5-b]pyridinium 3-oxide hexafluorophosphate (350.4 mg, 0.922 mmol, 1.7 eq.). The mixture was stirred at 20° C. for 2 hours. The reaction mixture was diluted with H₂O (5 mL) and extracted with EtOAc (5 mL×3). The combined organic layers were washed with brine (5 mL×3), dried over Na₂SO₄, filtered and concentrated under reduced pressure to give a residue. The residue was purified by prep-HPLC (Waters Xbridge Prep OBD C18 (75*30 mm*3 μm), water(NH₄HCO₃)-ACN, 35-65% B, 8 min) to afford the title compound (82.7 mg, 0.255 mmol, 47% yield) as a yellow oil. LC-MS m/z: 324.0 [M+1].

Following compounds were synthesized by Scheme 6 (Example 6) with appropriate reagents and purification methods:

| Compound # | LC-MS m/z: [M = 1] |
|---|---|
| 61 | 324.0 |
| 62 | 324.0 |
| 63 | 324.0 |
| 68 | 324.0 |
| 69 | 324.0 |
| 70 | 324.0 |
| 76 | 324.0 |

### Example 7. Synthesis of Compound 91

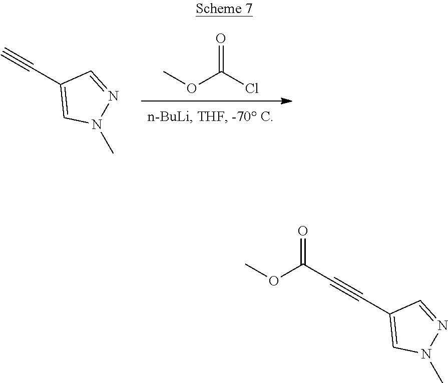

Step 1. To a solution of 4-ethynyl-1-methyl-pyrazole (200 mg, 1.88 mmol, 1 eq.) in THF (5 mL, 0.38 M) was added n-BuLi (0.9 mL, 2.5 M, 2.26 mmol, 1.2 eq.) at −70° C. and stirred for 30 minutes. Methyl chloroformate (430 mg, 4.55 mmol, 2.4 eq.) was added and the reaction was stirred at −70° C. for 1.5 hours. The reaction was quenched with water (15 mL) and extracted with ethyl acetate (15 mL×3). The combined organic layers were washed with brine (20 mL), dried over Na₂SO₄, filtered and concentrated to provide a residue. The residue was purified by prep-TLC (petroleum ether:ethyl acetate=3:1, Rf (product)=0.6 UV) to provide methyl 3-(1-methylpyrazol-4-yl)prop-2-ynoate (200 mg, 1.22 mmol, 64.7% yield) as a white solid.

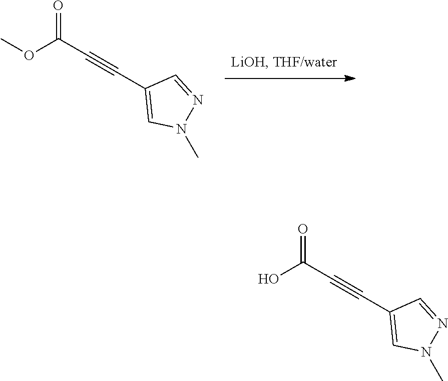

Step 2. To a solution of methyl 3-(1-methylpyrazol-4-yl)prop-2-ynoate (200 mg, 1.22 mmol, 1 eq.) in THF (3 mL) and water (1 mL) (0.3 M) was added lithium hydroxide (58.4 mg, 2.44 mmol, 2 eq.) at 25° C. for 1 hour. The reaction was then concentrated, diluted with water (5 mL), neutralized with aqueous HCl (1 M) to pH=3˜4, then extracted with ethyl acetate (5 mL×3), and the combined organic layers were washed with brine (10 mL) then dried over Na₂SO₄ and concentrated to provide 3-(1-methylpyrazole-4-yl)prop-2-ynoic acid (200 mg, 1.33 mmol, >100% yield) as a yellow solid which was used without further purification.

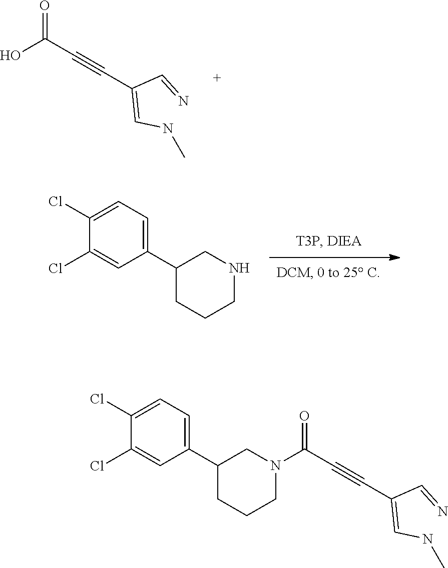 [SMILES: CN/C=C(/C#CC(=O)O)C=N ; Clc1ccc(C2CCCNC2)cc1Cl ; CN/C=C(/C#CC(=O)N1CCCC(c2ccc(Cl)c(Cl)c2)C1)C=N]

Step 3. To a solution of 3-(1-methylpyrazole-4-yl)prop-2-ynoic acid (130 mg, 0.57 mmol, 1 eq.) in DCM (5 mL, 0.17 M) was added 3-(3,4-dichlorophenyl)piperidine (199.3 mg, 0.87 mmol, I eq.) followed by DIEA (557 mg, 5 eq.) then T3P (1.37 g, 2.5 eq., 50% in ethyl acetate) at 0° C. The reaction was allowed to warm to 25° C. and stirred for 1.5 hours. The reaction mixture was diluted with water (15 mL), extracted with ethyl acetate (15 mL×3), and the combined organic layers were washed with brine (20 mL), dried over Na₂SO₄, filtered and concentrated to provide a residue. The residue was purified by prep-HPLC to provide the title compound (111 mg, 0.31 mmol, 35.5% yield) as a pale yellow solid. LC-MS m/z: 362.0 [M+1]

Following compounds were synthesized by Scheme 7 with appropriate reagents and purification methods:

Compound 92: LCMS m/z: 362.0 [M+1].

### Example 8. Synthesis of Compound 75

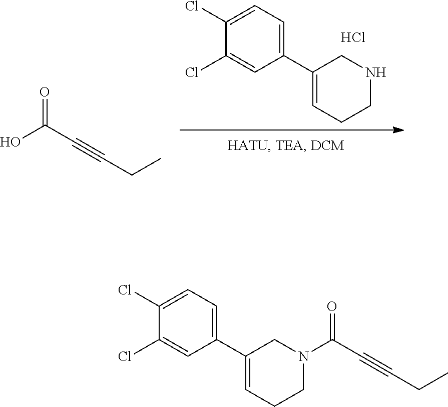

To a solution of pentynoic acid (70 mg, 0.714 mmol, 1 eq.) and 5-(3,4-dichlorophenyl)-1,2,3,6-tetrahydropyridine hydrochloride (208 mg, 0.78 mmol, 1.1 eq.) in DCM (5 mL, 0.14 M) was added triethylamine (216.62 mg, 2.14 mmol, 297.96 μL, 3 eq.) and (1-[Bis(dimethylamino)methylene]-1H-1,2,3-triazolo[4,5-b]pyridinium 3-oxide hexafluorophosphate (HATU, 407 mg, 1.07 mmol, 1.5 eq.) at 0° C. The mixture was stirred at 25° C. under N₂ for 4 hours. The reaction mixture was quenched by the addition of H₂O (20 mL) and extracted with CH₂Cl₂ (5 mL×3). The combined organic layers were washed with brine (20 mL), dried over Na₂SO₄, filtered and concentrated under reduced pressure to give a residue. The residue was purified by prep-HPLC (column: Phenomenex C18 150*25 mm*0 μm; mobile phase: [water (NH₄HCO₃)-ACN]; B %: 48%-78%, 5 min). 1-(5-(3,4-dichlorophenyl)-3,6-dihydropyridin-1(2H)-yl)pent-2-yn-1-one (37.1 mg, 0.12 mmol, 16.8% yield, 99.5% purity) was obtained as a white solid which was confirmed by LC-MS m/z: 308.0 [M+1].

### Example 9. Synthesis of Compound 81

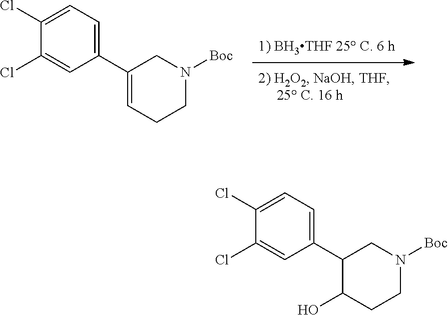 [SMILES: CC(C)(C)OC(=O)N1CCC=C(c2ccc(Cl)c(Cl)c2)C1 ; CC(C)(C)OC(=O)N1CCC(O)C(c2ccc(Cl)c(Cl)c2)C1]

Step 1. To a solution of tert-butyl 5-(3,4-dichlorophenyl)-3,6-dihydropyridine-1(2H)-carboxylate (0.2 g, 0.61 mmol, 1 eq.) in THF (2 mL, 0.31M) was added BH₃-THF (1 M, 1.22 mL, 2 eq.) dropwise at 0° C. The mixture was stirred at 0° C. for 1 hour, and then H₂O₂ (117 mg, 1.04 mmol, 99.5 μL, 30% purity, 1.7 eq) and NaOH (3 M, 142 μL, 0.7 eq) was added dropwise at 0° C. The resulting mixture was stirred at 25° C. for 16 hours. The reaction was quenched with saturated aqueous Na₂S2O₃ (10 mL) and diluted with water (20 mL). The aqueous layer was extracted with diethyl ether (5×10 mL), the combined organic layers were washed with a saturated aqueous solution of NH₄Cl (10 mL) then brine (5 mL), were dried over Na₂SO₄ and concentrated in vacuo to give a residue. The residue was purified by column chromatography (SiO₂, Petroleum ether/Ethyl acetate=3/1 to 1/1) to give tert-butyl 3-(3,4-dichlorophenyl)-4-hydroxypiperidine-1-carboxylate (70 mg, 0.20 mmol, 33% yield) as a colorless oil.

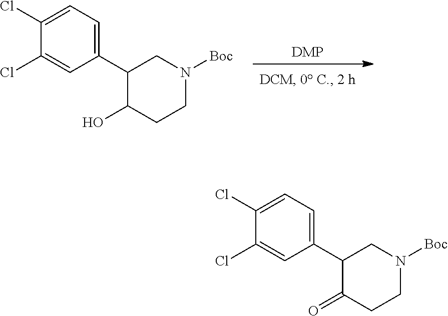

Step 2. To a solution of tert-butyl 3-(3,4-dichlorophenyl)-4-hydroxypiperidine-1-carboxylate (0.7 g, 2.02 mmol, 1 eq.) in DCM (8 mL, 0.2M) was added Dess-Martin periodinane (DMP, 1.29 g, 3.03 mmol, 939 μL, 1.5 eq) at 0° C. under N₂. The mixture was stirred at 0° C. for 2 hours under N₂. The solution was diluted with EtOAc (10 mL) and H₂O (5 mL) and extracted with EtOAc (10 mL×3). The combined organic layers were dried over Na₂SO₄. The solution was concentrated to provide a residue. The residue was purified by column chromatography (SiO₂, Petroleum ether/Ethyl acetate=3/1 to 1/1) to give tert-butyl 3-(3,4-dichlorophenyl)-4-oxopiperidine-1-carboxylate (0.4 g, 1.16 mmol, 57.5% yield) as a white solid.

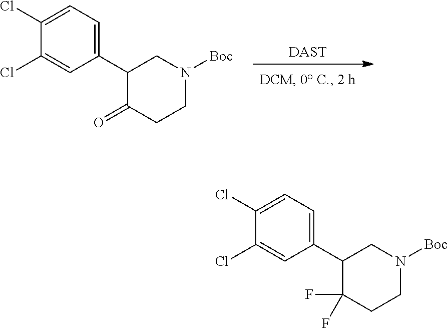 [SMILES: CC(C)(C)OC(=O)N1CCC(=O)C(c2ccc(Cl)c(Cl)c2)C1 ; "DAST" ; CC(C)(C)OC(=O)N1CCC(F)(F)C(c2ccc(Cl)c(Cl)c2)C1]

Step 3. To a solution of tert-butyl 3-(3,4-dichlorophenyl)-4-oxopiperidine-1-carboxylate (200 mg, 0.58 mmol, 1 eq.) in DCM (3 mL, 0.2 M) was added diethylaminosulfurtrifluoride (DAST, 187.3 mg, 1.16 mmol, 153.53 μL, 2 eq.) at 0° C. under N₂. The mixture was stirred at 0° C. for 1 hour under N₂. The solution was diluted with EtOAc (10 mL) and H₂O (5 mL) and extracted with EtOAc (10 mL×3). The combined organic layers were dried over Na₂SO₄ and concentrated to provide a residue. The residue was purified by prep-TLC (Petroleum ether/Ethyl acetate=3/1, Rf (product)=0.5) to give tert-butyl 3-(3,4-dichlorophenyl)-4,4-difluoropiperidine-1-carboxylate (0.2 g, 0.546 mmol, 94% yield) as a yellow oil.

Following Step 4 and 5 of Scheme I with tert-butyl 3-(3,4-dichlorophenyl)-4,4-difluoropiperidine-1-carboxylate, the title compound was obtained. LC-MS m/z: 346.0 [M+1].

### Example 10. Synthesis of Compound 82

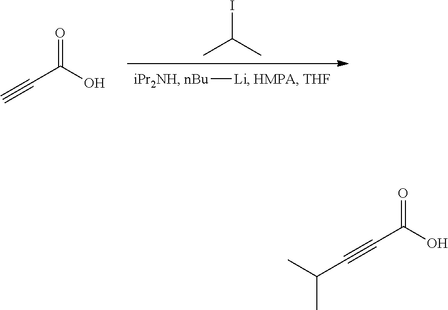

To a solution of N-isopropylpropan-2-amine (6.65 g, 65.67 mmol, 9.28 mL, 2.3 eq) in THF (12 mL) was added n-BuLi (2.5 M, 25.13 mL, 2.2 eq) dropwise at 0° C. under N₂ (to prepare LDA). After 0.5 hour, propiolic acid (2 g, 28.55 mmol, 1.75 mL, 1 eq) and HMPA (23.02 g, 128.49 mmol, 22.57 mL, 4.5 eq) and THF (10 mL) was added into above mixture at −40° C., and the reaction was stirred at −40° C. for 0.5 hour under N₂. 2-iodopropane (5.82 g, 34.26 mmol, 3.43 mL, 1.2 eq) in THF (8 mL) was added at −40° C. under N₂. Then the reaction was stirred at 25° C. for 13 hours. The reaction mixture was quenched by the addition of water (50 mL) and adjusted to pH=2˜3 with HCl (1 N). The resulting solution was extracted with ethyl acetate (30 mL×2). The combined organic layers were washed with brine (50 mL), dried over Na₂SO₄, filtered and concentrated under reduced pressure to give a residue. The residue was purified by column chromatography (SiO₂, Dichloromethane:Methanol=100/1˜2/1). 4-methylpent-2-ynoic acid (838 mg, 4.86 mmol, 17% yield, 65% purity) was obtained as yellow oil.

Following step 5 of Scheme 1 (Example 1) with 4-methylpent-2-ynoic acid, the title compound was obtained. LC-MS m/z: 324.0 [M+1].

### Example 11. Synthesis of Compound 83

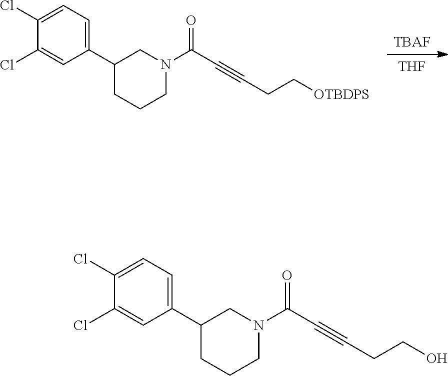

Step 1. To a solution of 5-((tert-butyldiphenylsilyl)oxy)-1-(3-(3,4-dichlorophenyl)piperidin-1-yl)pent-2-yn-1-one (0.7 g, 1.24 mmol, 1 eq) in THF (5 mL, 0.2 M) was added TBAF (1 M, 1.49 mL, 1.2 eq) at 0° C. under N₂. The mixture was stirred at 25° C. for 1 hour. The reaction mixture was quenched by the addition of H₂O (30 mL) and extracted with ethyl acetate (10 mL×3). The combined organic layers were washed with brine (30 mL), dried over Na₂SO₄, filtered and concentrated under reduced pressure to give a residue. The residue was purified by column chromatography (SiO₂, Petroleum ether:Ethyl acetate=100/1˜1/3). 1-(3-(3,4-dichlorophenyl)piperidin-1-yl)-5-hydroxypent-2-yn-1-one (0.3 g, 919.63 μmol, 74.18% yield) was obtained as a pale-yellow oil.

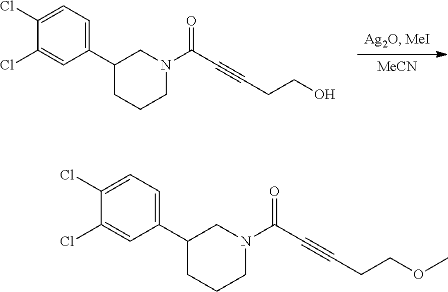

Step 2. To a solution of 1-(3-(3,4-dichlorophenyl)piperidin-1-yl)-5-hydroxypent-2-yn-1-one (0.3 g, 0.92 mmol, 1 eq.) in MeCN (10 mL, 0.09M) was added Ag₂O (426.2 mg, 1.84 mmol, 2 eq.) and Mel (652.7 mg, 4.60 mmol, 286.3 μL, 5 eq.). The mixture was stirred at 50° C. for 24 hours. The reaction mixture was filtered and concentrated under reduced pressure to give a residue. The residue was purified by prep-HPLC (column: Phenomenex C18 150*25 mm*10 μm; mobile phase: [water (NH₄HCO₃)-ACN]; B %: 44%-74%, 8 min). 1-(3-(3,4-dichlorophenyl)piperidin-1-yl)-5-methoxypent-2-yn-1-one (85.4 mg, 0.246 mmol, 26.7% yield, 97.9% purity) was obtained as yellow oil. LC-MS m/z: 399.8 [M+1].

### Example 12. Synthesis of Compound 85

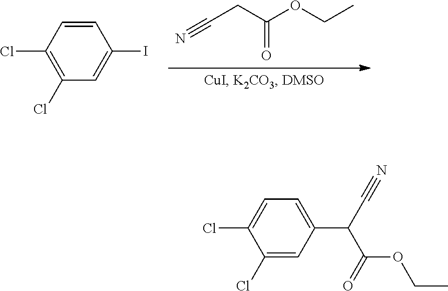

Step 1. To a solution of 1,2-dichloro-4-iodobenzene (15 g, 55 mmol, 1 eq.) and ethyl 2-cyanoacetate (6.2 g, 55 mmol, 1 eq.) in DMSO (75 mL, 0.73M) was added K₂CO₃ (30.4 g, 220 mmol, 4 eq) and CuI (1.05 g, 5.50 mmol, 0.1 eq). The mixture was stirred at 120° C. for 12 hours under N₂. The reaction mixture was filtered and diluted with H₂O (400 mL), adjusted to pH=2 by addition of aqueous HCl (1 N). The aqueous phase was extracted with Ethyl acetate (50 mL×3). The combined organic layers were washed with H₂O (50 mL×2) then brine (50 mL), dried over Na₂SO₄, and concentrated under reduced pressure to give a residue. The residue was purified by column chromatography (SiO₂, Petroleum ether/Ethyl acetate=100/1 to 5/1). Ethyl 2-cyano-2-(3,4-dichlorophenyl)acetate (12 g, 46.49 mmol, 84.59% yield) was obtained as a yellow oil.

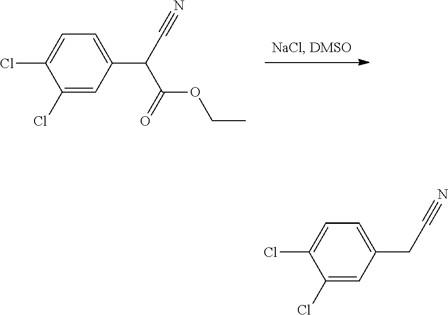

Step 2. To a solution of ethyl 2-cyano-2-(3,4-dichlorophenyl)acetate (15 g, 58 mmol, 1 eq.) in DMSO (150 mL, 0.39 M) was added NaCl (10.19 g, 174.35 mmol, 3 eq.) in H₂O (30 mL), then the mixture was stirred at 120° C. for 16 hours. The reaction mixture was filtered and diluted with H₂O (500 mL), adjusted to pH=2 by the addition of aqueous 1 N HCl. The aqueous phase was extracted with Ethyl acetate (100 mL×3). The combined organic phase was washed with H₂O (200 mL×2) then brine (100 mL), dried over Na₂SO₄, and concentrated under reduced pressure to give a residue. The residue was purified by column chromatography (SiO₂, Petroleum ether/Ethyl acetate=100/1 to 5/1). 2-(3,4-dichlorophenyl)acetonitrile (9.4 g, 50.53 mmol, 87% yield) was obtained as a yellow solid.

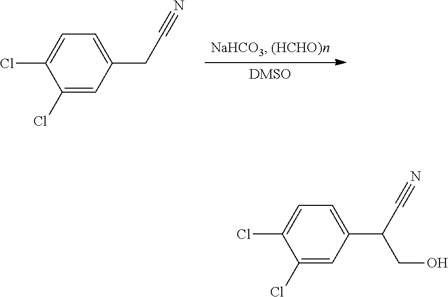

Step 3. To a solution of 2-(3,4-dichlorophenyl)acetonitrile (3.4 g, 18.28 mmol, 1 eq) and NaHCO₃ (153.54 mg, 1.83 mmol, 0.1 eq) in DMSO (30 mL, 0.6 M) was added (HCHO)ₙ (576.20 mg, 6.40 mmol, 0.35 eq) at 25° C. under N₂. The reaction mixture was stirred at 25° C. for 2 hours. The reaction mixture was filtered and diluted with H₂O (150 mL), adjusted to pH=2 by the addition of aqueous 1N HCl. The aqueous phase was extracted with ethyl acetate (50 mL×3). The combined organic phase was washed with brine (50 mL), dried over Na₂SO₄, concentrated under reduced pressure to give a residue. The residue was purified by column chromatography (SiO₂, Petroleum ether/Ethyl acetate=100/1 to 4/1). 2-(3,4-dichlorophenyl)-3-hydroxypropanenitrile (1.85 g, 8.56 mmol, 47% yield) was obtained as a yellow oil.

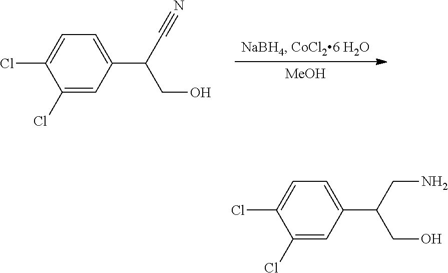 [SMILES: "MeOH" ; N#CC(CO)c1ccc(Cl)c(Cl)c1 ; "NaBH4, CoCl2·6 H2O" ; NCC(CO)c1ccc(Cl)c(Cl)c1]

Step 4. To a solution of 2-(3,4-dichlorophenyl)-3-hydroxypropanenitrile (1.2 g, 5.5 mmol, 1 eq.) and CoCl2·6H₂O (1.72 g, 7.2 mmol, 1.3 eq.) in MeOH (12 mL 0.46M) was added NaBH₄ (630 mg, 16.7 mmol, 3 eq.) at 0° C. under N₂. The reaction mixture was stirred at 25° C. for 12 hours. The reaction mixture was diluted with Ethyl acetate (10 mL) and H₂O (15 mL), and the organic layer was separated. The aqueous phase was extracted with ethyl acetate (15 mL×3). The combined organic layers were washed with brine (30 mL), dried over Na₂SO₄, and concentrated under reduced pressure to give a residue which was carried forward without further purification. 3-amino-2-(3,4-dichlorophenyl)propan-1-ol (1.3 g, 5.49 mmol, 98.90% yield, 93% purity) was obtained as a yellow oil.

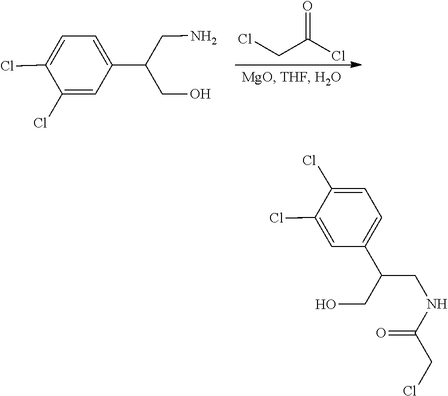 [SMILES: NCC(CO)c1ccc(Cl)c(Cl)c1 ; O=C(Cl)CCl ; O=C(CCl)NCC(CO)c1ccc(Cl)c(Cl)c1]

Step 5. To a solution of 3-amino-2-(3,4-dichlorophenyl)propan-1-ol (2.5 g, 11.36 mmol, 1 eq.) and MgO (2.06 g, 51.11 mmol, 575.39 L, 4.5 eq.) in THF (35 mL) and H₂O (7 mL) (0.27 M) was added chloroacetyl chloride (2.95 g, 26.13 mmol, 2.08 mL, 2.3 eq) at 0° C. The reaction mixture was stirred at 25° C. for 1 hour. The reaction mixture was filtered, and the filtrate was washed with brine, dried over Na₂SO₄, concentrated under reduced pressure to give a residue which was carried forward without further purification. 2-chloro-N-(2-(3,4-dichlorophenyl)-3-hydroxypropyl)acetamide (2.2 g, 7.42 mmol, 65.31% yield) was obtained as a green solid.

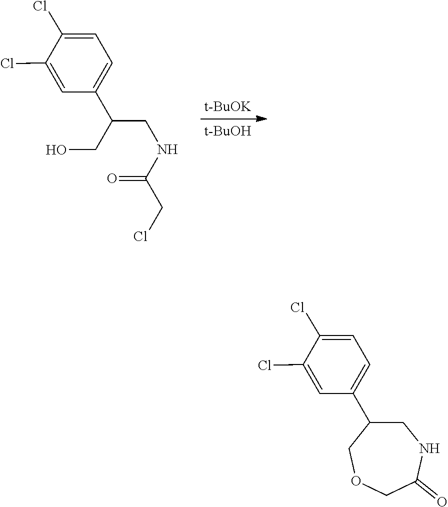 [SMILES: "t-BuOK" ; "t-BuOH" ; O=C(CCl)NCC(CO)c1ccc(Cl)c(Cl)c1 ; O=C1COCC(c2ccc(Cl)c(Cl)c2)CN1]

Step 6. To a solution of 2-chloro-N-(2-(3,4-dichlorophenyl)-3-hydroxypropyl)acetamide (2.1 g, 7.08 mmol, 1 eq) in t-BuOH (45 mL, 0.16 M) was added t-BuOK (1.59 g, 14.16 mmol, 2 eq) at 25° C. The reaction mixture was stirred at 25° C. for 6 hours. The reaction mixture was diluted with H₂O (30 mL) then adjusted to pH=7 with aqueous 1 N HCl. The mixture was extracted with DCM (30 mL×2). The combined organic layers were washed with brine (50 mL), dried over Na₂SO₄, and concentrated under reduced pressure to give a residue. The residue was purified by column chromatography (SiO₂, Petroleum ether/Ethyl acetate=100/1 to 0/1). 6-(3,4-dichlorophenyl)-1,4-oxazepan-3-one (0.48 g, 1.85 mmol, 26.1% yield) was obtained as a yellow solid.

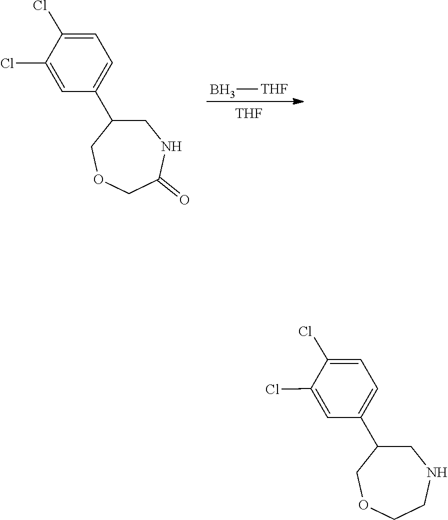

Step 7. To a solution 6-(3,4-dichlorophenyl)-1,4-oxazepan-3-one (0.48 g, 1.85 mmol, 1 eq.) in THF (10 mL, 1.85 M) was added BH₃-THF (1 M, 7.38 mL, 4 eq) at 0° C. under N₂. The reaction mixture was stirred at 0° C. for 2 hours. The reaction mixture was poured into MeOH (74 mL) and stirred at 75° C. for 3 hours. The reaction mixture was then concentrated under reduced pressure to remove the MeOH. The residue was diluted with H₂O (10 mL), adjusted to pH=2 with aqueous 1N HCl. The mixture was extracted with Ethyl acetate (10 mL×2). The aqueous phase was concentrated under vacuum to give a residue, which was carried forward without further purification. 6-(3,4-dichlorophenyl)-1,4-oxazepane (0.19 g, 672.34 μmol, 36.43% yield, HCl) was obtained as a yellow solid.

Following step 5 of Scheme 1 (Example 1) with 6-(3,4-dichlorophenyl)-1,4-oxazepane, the title compound was obtained. LC-MS m/z: 326.0 [M+1].

### Example 13. Synthesis of Compound 86

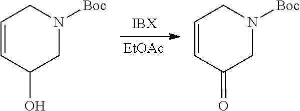 [SMILES: "EtOAc" ; CC(C)(C)OC(=O)N1CC=CC(O)C1 ; CC(C)(C)OC(=O)N1CC=CC(=O)C1]

Step 1. To a solution of tert-butyl 3-hydroxy-3,6-dihydropyridine-1(2H)-carboxylate (0.7 g, 3.51 mmol, 1 eq) in ethyl acetate (15 mL) was added IBX (1.97 g, 7.03 mmol, 2 eq.) at 25° C. The reaction mixture was stirred at 60° C. for 12 hours. The reaction mixture was diluted with ethyl acetate (10 mL) and H₂O (15 mL), and the organic layers were separated. The aqueous phase was extracted with Ethyl acetate (10 mL×3). The combined organic layers were washed with brine (30 mL), dried over Na₂SO₄, concentrated under reduced pressure to give a residue. The residue was purified by column chromatography (SiO2, Petroleum ether/Ethyl acetatc=100/1 to 3/1). Tert-butyl 3-oxo-3,6-dihydropyridine-1(2H)-carboxylate (0.61 g, 3.09 mmol, 88.03% yield) was obtained as a white oil.

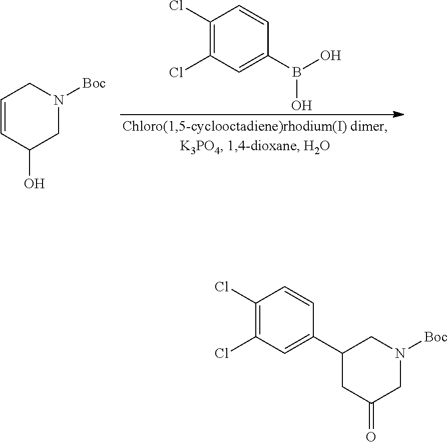 [SMILES: CC(C)(C)OC(=O)N1CC=CC(O)C1 ; OB(O)c1ccc(Cl)c(Cl)c1 ; "Chloro(1,5-cyclooctadiene)rhodium(I) dimer,K3PO4, 1,4-dioxane, H2O" ; CC(C)(C)OC(=O)N1CC(=O)CC(c2ccc(Cl)c(Cl)c2)C1]

Step 2. To a solution of tert-butyl 3-oxo-3,6-dihydropyridine-1(2H)-carboxylate (0.7 g, 3.55 mmol, 1 eq.) and (3,4-dichlorophenyl)boronic acid (1.02 g, 5.32 mmol, 1.5 eq.) in 1,4-dioxane (15 mL, 0.24) was added K₃PO₄ (1.5 M, 2.84 mL, 1.2 eq.) and chlororhodium(1Z,5Z)-cycloocta-1,5-diene (52.50 mg, 106.47 μmol, 0.03 eq). The mixture was stirred at 25° C. under N₂ for 3 hours. The mixture was diluted with Ethyl acetate (30 mL) and H₂O (20 mL). The organic layer was separated, washed with brine (20 mL), concentrated under reduced pressure. The residue was purified by column chromatography (SiO₂, Petroleum ether/Ethyl acetate=100/1 to 5/1). tert-butyl 3-(3,4-dichlorophenyl)-5-oxopiperidine-1-carboxylate (0.75 g, 2.18 mmol, 61.39% yield) as a yellow solid.

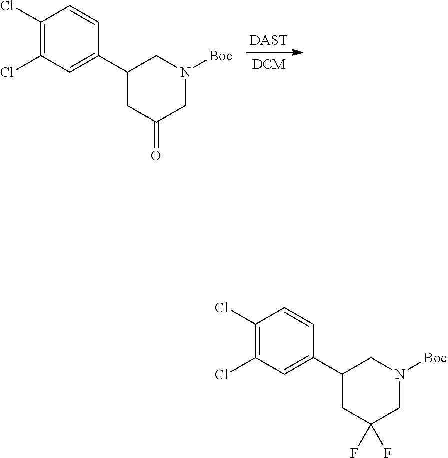

Step 3. To a solution tert-butyl 3-(3,4-dichlorophenyl)-5-oxopiperidine-1-carboxylate (0.64 g, 1.86 mmol, 1 eq) in Dichloromethane (10 mL, 0.19 M) was added diethylamino sulfur trifluoride (659.31 mg, 4.09 mmol, 540.42 μL, 2.2 eq) at 0° C. under N₂. The reaction mixture was stirred under N₂ at 0° C. for 2 hours. The reaction mixture was poured into NaHCO₃ aq. (10 mL) and the pH maintained at 7-8. The aqueous phase was extracted with dichloromethane (10 mL×3). The combined organic phase was washed with brine (30 mL), dried over Na₂SO₄, concentrated under reduced pressure to give a residue. The aqueous phase was extracted with dichloromethane (10 mL×3). The combined organic phase was washed with brine (30 mL), dried over Na₂SO₄, concentrated under reduced pressure to give a residue. The residue was purified by column chromatography (SiO₂, Petroleum ether/Ethyl acetate=100/1 to 3/1). tert-butyl 5-(3,4-dichlorophenyl)-3,3-difluoropiperidine-1-carboxylate (0.13 g, 354.97 μmol, 19.09% yield) was obtained as yellow oil.

Following Step 4 and 5 of Scheme 1 (Example 1) with tert-butyl 3-(3,4-dichlorophenyl)-5,5-difluoropiperidine-1-carboxylate, the title compound was obtained. LC-MS m/z: 345.9 [M+1].

### Example 14. Synthesis of Compound 89

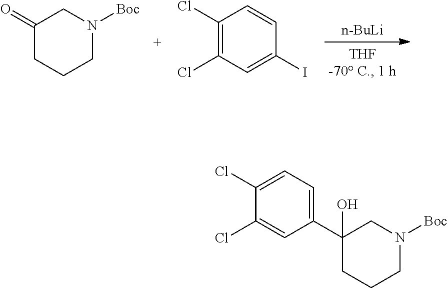

Step 1. To a solution of 1,2-dichloro-4-iodo-benzene (1 g, 3.665 mmol, 1 eq.) in THF (10 mL, 0.3664 M) was added dropwise n-butyllithium solution (1.61 mL, 4.031 mmol, 2.5 M, 1.1 eq.) at −70° C. After 1 hour, a solution of tert-butyl 3-oxopiperidine-1-carboxylate (803.2 mg, 4.031 mmol, 1.1 eq.) in THF (5 mL) was added dropwise at −70° C. The mixture was stirred at −70° C. for 1 hour under N₂. The reaction mixture was poured into H₂O (15 mL) and extracted with DCM (15 mL×3). The combined organic layers were washed with brine (10 mL×2), dried over Na₂SO₄, filtered and concentrated under reduced pressure to give a residue. The residue was purified by column (SiO₂, petroleum ether:ethyl acetate=1:0 to 1:1) to give tert-butyl 3-(3,4-dichlorophenyl)-3-hydroxy-piperidine-1-carboxylate (600 mg, 1.73 mmol, 47% yield) as a yellow solid.

Following Step 4 and 5 of Scheme 1 (Example 1) with tert-butyl 3-(3,4-dichlorophenyl)-3-hydroxy-piperidine-1-carboxylate, the title compound was obtained. LC-MS m/z: 326.2 [M+1].

### Example 15. Synthesis of Compound 103

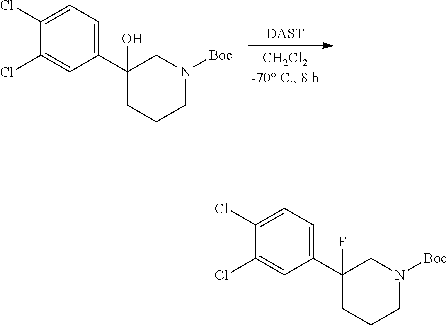

Step 1. To a solution of tert-butyl 3-(3,4-dichlorophenyl)-3-hydroxy-piperidine-1-carboxylate (100 mg, 0.289 mmol, 1 eq.) in DCM (10 mL, 0.0289 M) was added diethylaminosulfur trifluoride (223.5 mg, 1.386 mmol, 4.8 eq.). The mixture was stirred at −70° C. for 8 hours. The reaction mixture was poured into aqueous NaHCO₃ (20 mL) and extracted with DCM (15 mL×3). The combined organic layers were washed with brine (10 mL×2), dried over Na₂SO₄, filtered and concentrated under reduced pressure to give a residue. The residue was purified by prep-TLC (SiO₂, petroleum ether:ethyl acetate=3:1) to give tert-butyl 3-(3,4-dichlorophenyl)-3-fluoro-piperidine-1-carboxylate (50 mg, 0.144 mmol, 50% yield) as a yellow oil.

Following Step 4 and 5 of Scheme 1 with tert-butyl 3-(3,4-dichlorophenyl)-3-fluoro-piperidine-1-carboxylate, the title compound was obtained. LC-MS m/z: 328.2 [M+1].

### Example 16. Synthesis of Compound 102

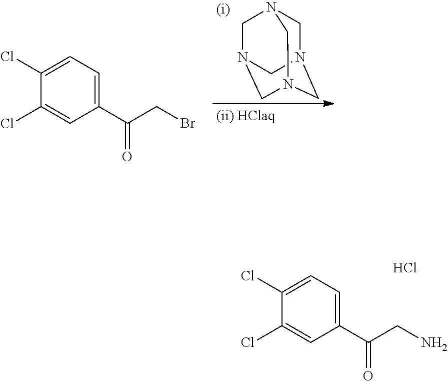 [SMILES: O=C(CBr)c1ccc(Cl)c(Cl)c1 ; C1N2CN3CN1CN(C2)C3 ; Cl ; NCC(=O)c1ccc(Cl)c(Cl)c1]

Step 1. To a solution of 2-bromo-1-(3,4-dichlorophenyl)ethanone (15 g, 55.99 mmol, 1 eq.) in ethanol (150 mL, 0.373 M) was added hexamethylenetetramine (9 g, 1.2 eq.) and NaI (8.19 g, 1 eq.). The mixture was stirred at 20° C. for 12 hours. The reaction mixture was concentrated under reduced pressure to give a residue. The residue was taken up in EtOH (150 mL) and added 4 N HCl (15 mL). The mixture was stirred at 50° C. for 3 hours. The formed precipitate was collected via filtration, taken up in H₂O, filtered and concentrated under reduced pressure to give 2-amino-1-(3,4-dichlorophenyl)ethanone; hydrochloride (11.0 g, 45.7 mmol, 82% yield) as a yellow solid.

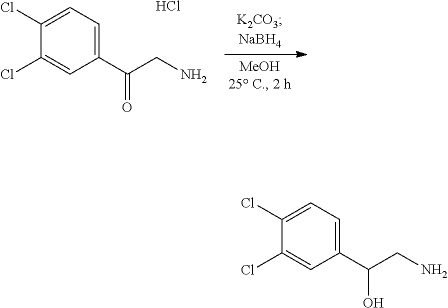 [SMILES: Cl ; NCC(=O)c1ccc(Cl)c(Cl)c1 ; NCC(O)c1ccc(Cl)c(Cl)c1]

Step 2. To a solution of 2-amino-1-(3,4-dichlorophenyl)ethanone; hydrochloride (1 g, 4.158 mmol, 1 eq.) in methanol (10 mL, 0.416 M) was added potassium carbonate (574.6 mg, 4.158 mmol, 1 eq.). The mixture was stirred for 5 min at 25° C. Sodium borohydride (314.6 mg, 8.316 mmol, 2 eq.) was added to the mixture at 0° C. under N₂. The mixture was stirred for 2 hours at 25° C. The reaction mixture was quenched with water (10 mL) and extracted with DCM (20 mL×2). The combined organic layer was washed with brine (10 mL) and dried over Na₂SO₄, filtered and concentrated under reduced pressure to give crude 2-amino-1-(3,4-dichlorophenyl)ethanol (0.80 g, 3.88 mmol, 93% yield) as a colorless oil.

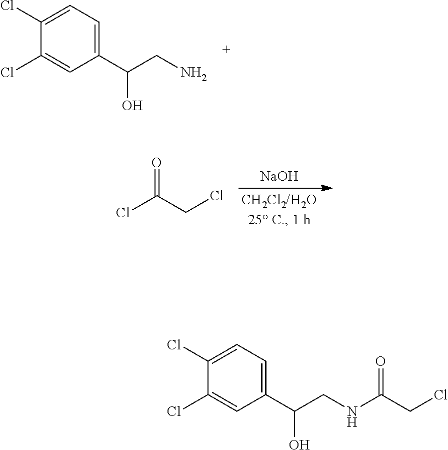

Step 3. To a solution of 2-amino-1-(3,4-dichlorophenyl)ethanol (500 mg, 2.426 mmol, 1 eq.) in DCM (10 mL, 0.121 M) and water (10 mL, 0.121 M) was added NaOH (116.5 mg, 1.2 eq.) and 2-chloroacetyl chloride (411.1 mg, 3.64 mmol, 1.5 eq.) at 0° C. The mixture was stirred at 25° C. for 1 hour. The mixture was added aqueous NaHCO₃ solution to adjust pH˜7, quenched with water (10 mL) and extracted with DCM (20 mL×2). The combined organic layer was washed with brine (10 mL) and dried over Na₂SO₄, filtered and concentrated under reduced pressure to give crude 2-chloro-N-[2-(3,4-dichlorophenyl)-2-hydroxy-ethyl]acetamide (600 mg, 2.12 mmol, 88% yield) as a yellow oil.

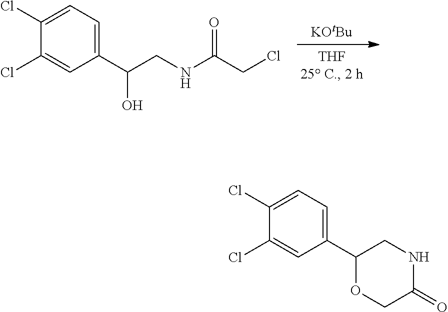 [SMILES: O=C(CCl)NCC(O)c1ccc(Cl)c(Cl)c1 ; O=C1COC(c2ccc(Cl)c(Cl)c2)CN1 ; "KOtBu"]

Step 4. To a solution of 2-chloro-N-[2-(3,4-dichlorophenyl)-2-hydroxy-ethyl]acetamide (600 mg, 2.124 mmol, 1 eq.) in THF (10 mL, 0.212 M) was added potassium tert-butoxide (285.9 mg, 2.548 mmol, 1.2 eq.). The mixture was stirred at 25° C. for 2 hours. The reaction mixture was quenched with water (10 mL) and extracted with EtOAc (20 mL×2). The combined organic layers were washed with brine (10 mL) and dried over Na₂SO₄, filtered and concentrated under reduced pressure to give a residue. The residue was purified by prep-TLC (ethyl acetate:MeOH=20:1) to give 6-(3,4-dichlorophenyl)morpholin-3-one (200 mg, 0.813 mmol, 38% yield) as a yellow oil.

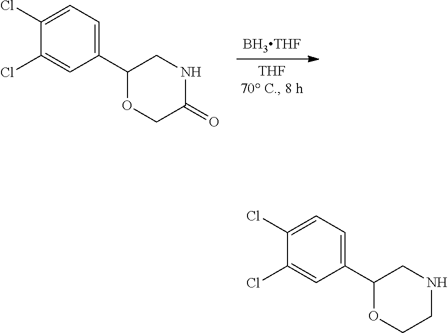 [SMILES: O=C1COC(c2ccc(Cl)c(Cl)c2)CN1 ; Clc1ccc(C2CNCCO2)cc1Cl ; "BH3·THF"]

Step 5. To a solution of 6-(3,4-dichlorophenyl)morpholin-3-one (200 mg, 0.813 mmol, 1 eq.) in THF (10 mL, 0.0813 M) was added BH₃/THF(1 M, 3 eq, 2.4 mL) under N₂ at 0° C. The mixture was stirred at 70° C. for 8 hours. The mixture was added MeOH (10 mL) and was stirred at 70° C. for 2 hours. The mixture was concentrated under reduced pressure to give crude 2-(3,4-dichlorophenyl)morpholine (150 mg) as a yellow oil.

Following Step 5 of Scheme 1 (Example 1) with 2-(3,4-dichlorophenyl)morpholine, the title compound was obtained. LC-MS m/z: 312.1 [M+1].

### Example 17. Synthesis of Compounds 112 and 113

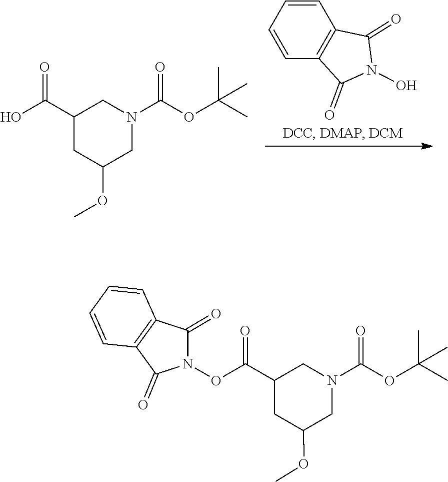

Step 1. To a solution of 1-(tert-butoxycarbonyl)-5-methoxypiperidine-3-carboxylic acid (260 mg, 1.0 mmol, 1 eq.) and DCC (207 mg, 1.0 mmol, 1 eq.) in DCM (20 mL, 0.05 M) was added DMAP (13 mg, 0.1 mmol, 0.1 eq.) at 25° C. The mixture was stirred at 25° C. under N₂ for 1 hour, then N-hydroxyphthalimide (0.18 g, 1.1 mmol, 1.1 eq.) was added and the reaction was stirred for an additional 4 hours. The reaction mixture was filtered and concentrated under reduced pressure to give a residue. The residue was purified by column chromatography (SiO₂, Petroleum ether/Ethyl acetate=3/1 to 1/100) to 1-(tert-butyl) 3-(1,3-dioxoisoindolin-2-yl) 5-methoxypiperidine-1,3-dicarboxylate (280 mg, 0.69 mmol, 69% yield) as a white solid.

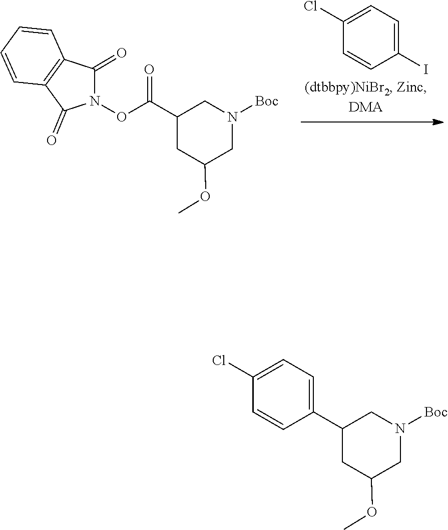 [SMILES: COC1CC(C(=O)ON2C(=O)c3ccccc3C2=O)CN(C(=O)OC(C)(C)C)C1 ; Clc1ccc(I)cc1 ; COC1CC(c2ccc(Cl)cc2)CN(C(=O)OC(C)(C)C)C1]

Step 2. To a solution 4-tert-butyl-2-(4-tert-butyl-2-pyridyl)pyridine nickel dibromide (20 mg, 0.06 mmol, 0.11 eq.) in DMA (2 mL, 0.27M) was added 1-(tert-butyl) 3-(1,3-dioxoisoindolin-2-yl) 5-methoxypiperidine-1,3-dicarboxylate (220 mg, 0.54 mmol, 1 eq.) and 1-chloro-4-iodobenzene (129.7 mg, 0.54 mmol, 1 eq.) and the resulting solution was stirred for 1 minute at 25° C. Zinc (100 mg, 1.53 mmol, 2.83 eq.) was added and the reaction was stirred at 25° C. for 20 hours. The mixture was diluted with MTBE (10 mL), filtered and the filter cake washed with additional MTBE (100 mL). The combined organic layers were washed with water (15 mL×2), dried over Na₂SO₄, filtered and concentrated to provide a residue. The residue was purified by column chromatography (SiO₂, Petroleum ether/Ethyl acetate 0 to 100%) to give tert-butyl 3-(4-chlorophenyl)-5-methoxypiperidine-1-carboxylate (50 mg, 0.15 mmol, 28% yield) as a colorless oil.

Step 4 and Step 5 of Scheme 1 (Example 1) with tert-butyl 3-(4-chlorophenyl)-5-methoxypiperidine-1-carboxylate the title compounds were obtained:

Compound 112: LC-MS m/z: 292.1 [M+1]

Compound 113: LC-MS m/z: 292.1 [M+1]

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A compound of Formula (II), or a pharmaceutically acceptable salt or solvate thereof: 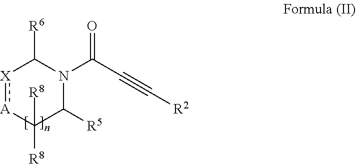
wherein: [symbol]
is a single or double bond;
X is C(J)(R⁷) when [symbol] is a single bond and C(J) when [symbol] is a double bond;
A is CR², NR¹, O or S or S(═O)₂ when [symbol] is a single bond; and CR when [symbol] is a double bond;
each R independently is H, halo, or C₁-C₆ alkyl;
R¹ is selected from the group C₁-C₆ alkyl, S(═O)₂—(C₁-C₆)alkyl, S(═O)₂—(C₆-C₁₀) aryl, C(═O)—(C₁-C₆) alkyl, or C(═O)—(C₆-C₁₀)aryl;
J is a C₆-C₁₀ aryl or a five- to six-membered heteroaryl, each of which is optionally substituted with 1-4 R³
each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halo, C₁-C₆ haloalkyl, cyano and N(R⁴)₂; or two R³ optionally are taken together with the carbon atoms to which they are attached to form a five- or six-membered heterocycle or a C₃-C₆ cycloalkyl;
R² is selected from the group consisting of C₆-C₁₀ aryl, five- or six-membered heteroaryl, C₁-C₆ alkyl, C₃-C₈ cycloalkyl, C₁-C₆ alkyl-hydroxy, C₁-C₆ alkoxy, C₁-C₆ alkyl-amino, C₂-C₆ alkenyl, C₂-C₆ alkynyl, C₁-C₆ haloalkyl, or four- to six-membered heterocyclyl, each of which is optionally substituted with 1-3 R⁴;
each R⁴ is independently selected from the group consisting of hydroxyl, C₁-C₆ alkoxy, C₁-C₆ alkyl, C(═O)R^{4a} or S(═O)₂R^{4a} or wherein two R⁴ optionally are taken together with the carbon atoms to which they are attached to form a five- to six-membered heterocyclyl or a C₃-C₆ cycloalkyl, wherein each R^{4a} independently is hydrogen or C₁-C₆ alkyl;
R⁵ is selected from the group consisting of hydrogen, C₁-C₆ alkyl, hydroxy, C₁-C₆ alkoxy, halogen, and —N(R³)₂;
R⁶ is H or C₁-C₆ alkyl;
R⁷ is H, hydroxy, or C₁-C₆ alkyl;
each R⁸ independently is H, halo, C₁-C₆ alkoxy, or C₁-C₆ alkyl; and
n is 0, 1, or 2;
or a compound selected from the group consisting of 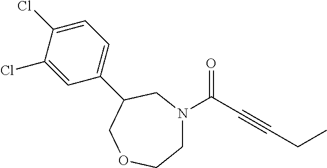 [SMILES: CCC#CC(=O)N1CCOCC(c2ccc(Cl)c(Cl)c2)C1]
or a pharmaceutically acceptable salt thereof.

2. The compound of claim 1 or a pharmaceutically acceptable salt or solvate thereof, wherein:
in J:
the optionally substituted C₆-C₁₀ aryl is phenyl which is optionally substituted with 1-4 R³;
the optionally substituted five- to six-membered heteroaryl is selected from the group consisting of pyridyl, pyrazolyl, and pyrimidinyl, each of which is optionally substituted with 1-4 R³;
in R¹:
the C₁-C₆ alkyl is methyl;
the C₁-C₆ alkyl in S(═O)₂—(C₁-C₆)alkyl is methyl;
the C₆-C₁₀ aryl in S(═O)₂—(C₆-C₁₀) aryl is phenyl;
the C₁-C₆ alkyl in C(═O)—(C₁-C₆) alkyl is methyl; and
the C₆-C₁₀ aryl in C(═O)—(C₆-C₁₀) aryl is phenyl;
in R³:
the C₁-C₆ alkyl is selected from the group consisting of methyl, and ethyl;
the C₃-C₆ cycloalkyl is cyclopropyl;
the C₁-C₆ alkoxy is methoxy; and
the C₁-C₆ haloalkyl is trifluoromethyl;
in R²:
the four- to six-membered heterocyclyl is selected from the group consisting of azetidinyl, tetrahydrofuranyl, piperidinyl, and pyrrolidinyl, each of which is optionally substituted with 1-3 R⁴;
the C₆-C₁₀ aryl is phenyl which is optionally substituted with 1-3 R⁴;
the C₃-C₈ cycloalkyl is selected from the group consisting of cyclopropyl, cyclobutyl, and cyclopentyl, each of which is optionally substituted with 1-3 R⁴;
the five- to six-membered heteroaryl is pyrazolyl which is optionally substituted with 1-3 R⁴;
the C₁-C₆ alkyl is selected from the group consisting of methyl, ethyl, and isopropyl, which is optionally substituted with 1-3 R⁴;
the C₁-C₆ alkyl-hydroxy is hydroxymethyl; and
the C₁-C₆ alkyl-amino is —CH₂—CH₂—NH—;
in R⁴:
the R^{4a} in S(═O)₂R^{4a} is selected from the group consisting of methyl and phenyl; and
the R^{4a} in C(═O)R^{4a} is methyl;
in R⁵:
the C₁-C₆ alkyl is methyl;
in R⁶:
the C₁-C₆ alkyl is methyl; and
in R⁸:
the C₁-C₆ alkyl is methyl; and
the C₁-C₆ alkoxy is methoxy.

3. (canceled)

4. (canceled)

5. The compound of claim 2, or a pharmaceutically acceptable salt or solvate thereof, wherein: A is CR², NR¹, or O.

6. The compound of claim 5, or a pharmaceutically acceptable salt or solvate thereof, wherein: n is 0, 1, or 2.

7-12. (canceled)

13. The compound of claim 2, selected from the group consisting of: 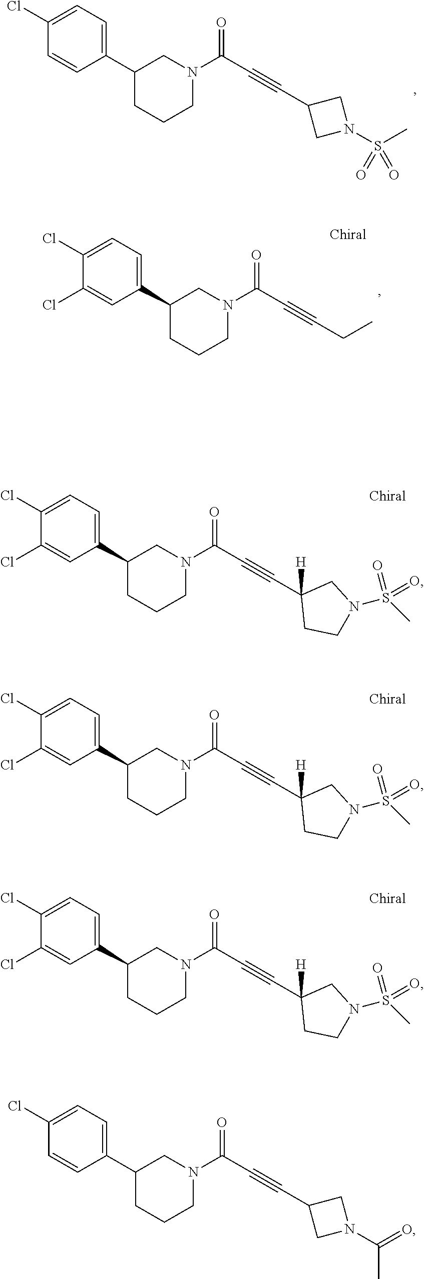 [SMILES: CS(=O)(=O)N1CC(C#CC(=O)N2CCCC(c3ccc(Cl)cc3)C2)C1 ; CCC#CC(=O)N1CCC[C@@H](c2ccc(Cl)c(Cl)c2)C1 ; CS(=O)(=O)N1CC[C@H](C#CC(=O)N2CCC[C@@H](c3ccc(Cl)c(Cl)c3)C2)C1 ; CS(=O)(=O)N1CC[C@H](C#CC(=O)N2CCC[C@@H](c3ccc(Cl)c(Cl)c3)C2)C1 ; CS(=O)(=O)N1CC[C@H](C#CC(=O)N2CCC[C@@H](c3ccc(Cl)c(Cl)c3)C2)C1 ; CC(=O)N1CC(C#CC(=O)N2CCCC(c3ccc(Cl)cc3)C2)C1] 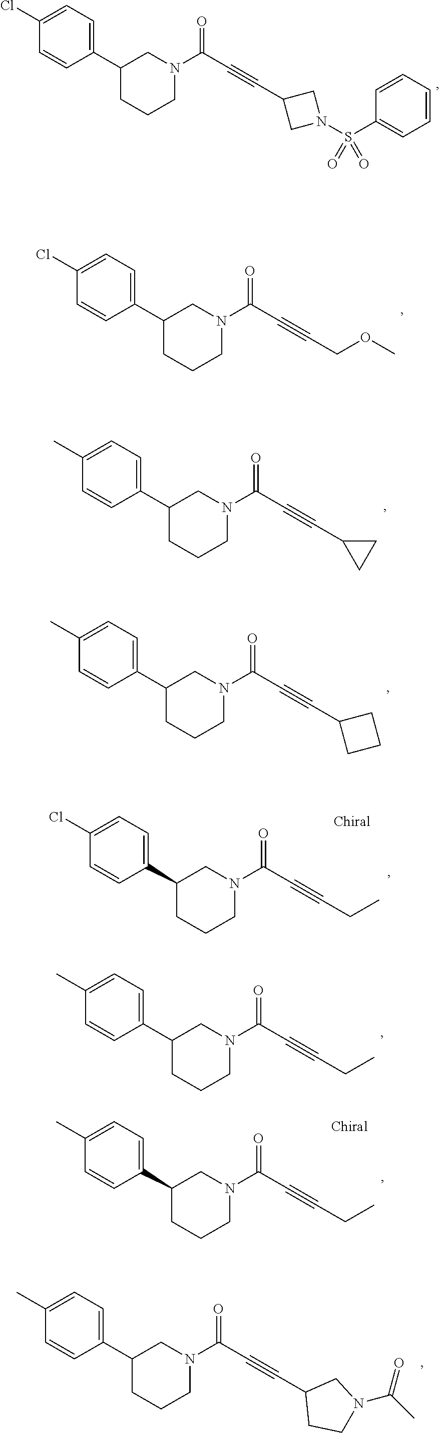 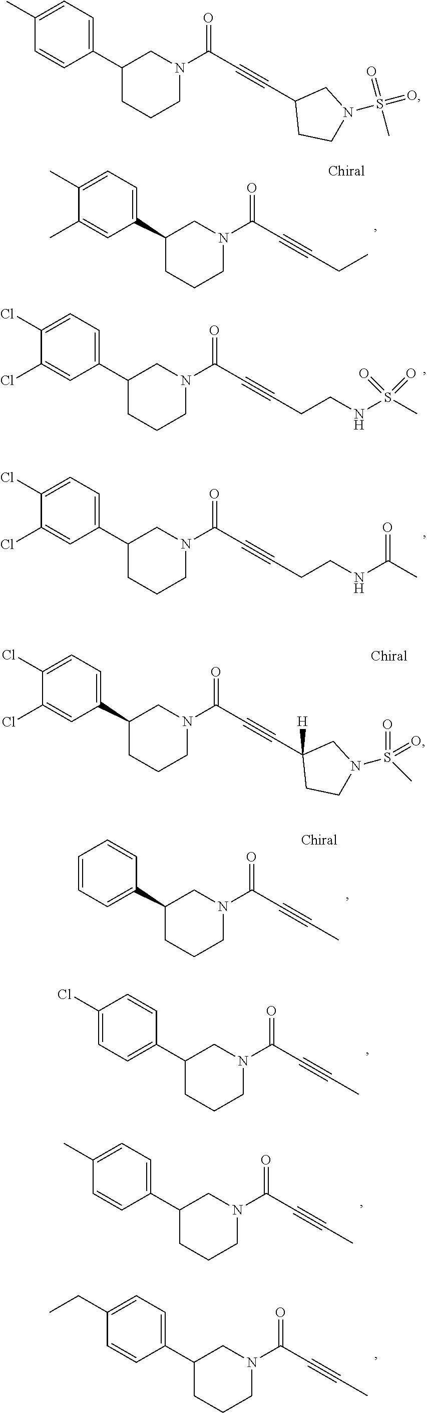 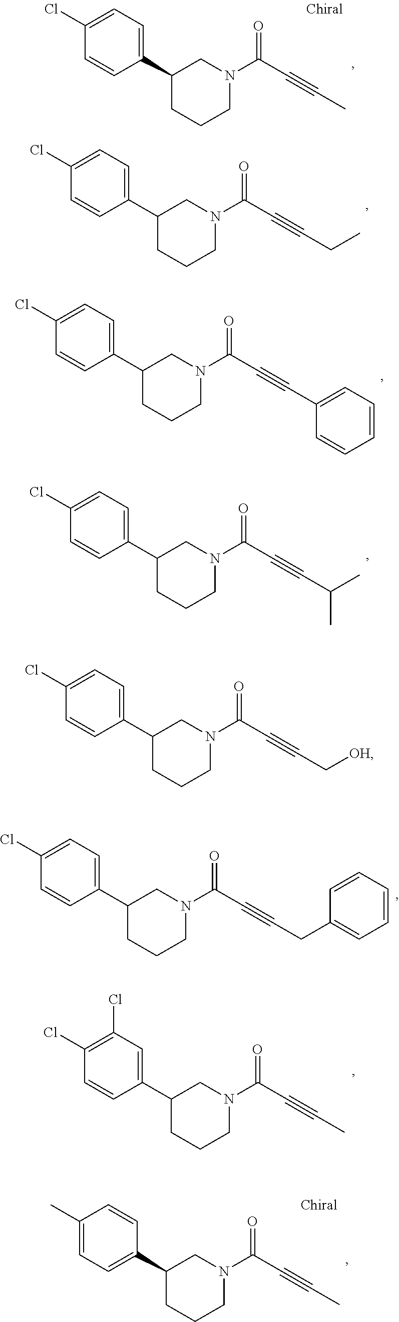 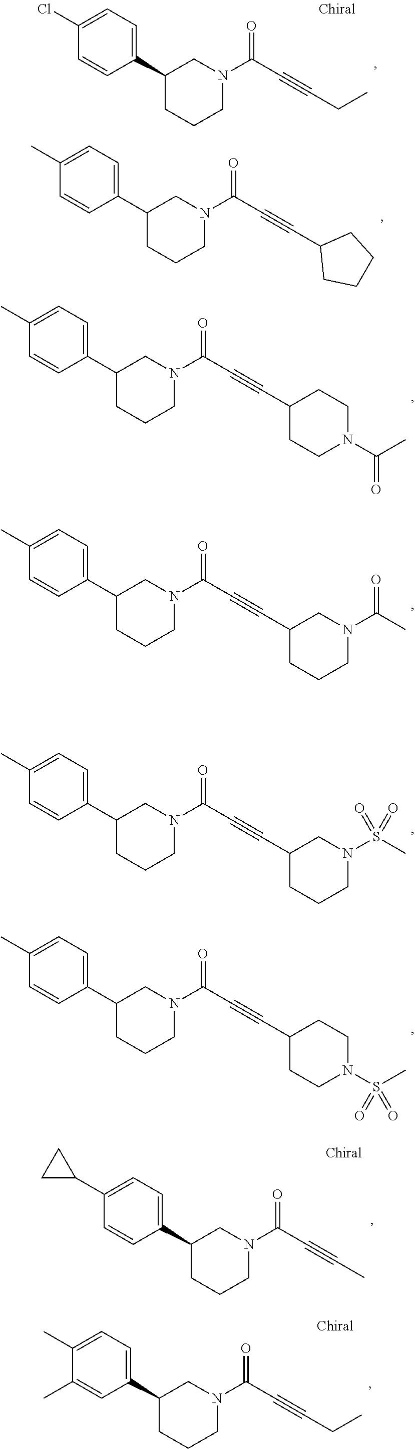 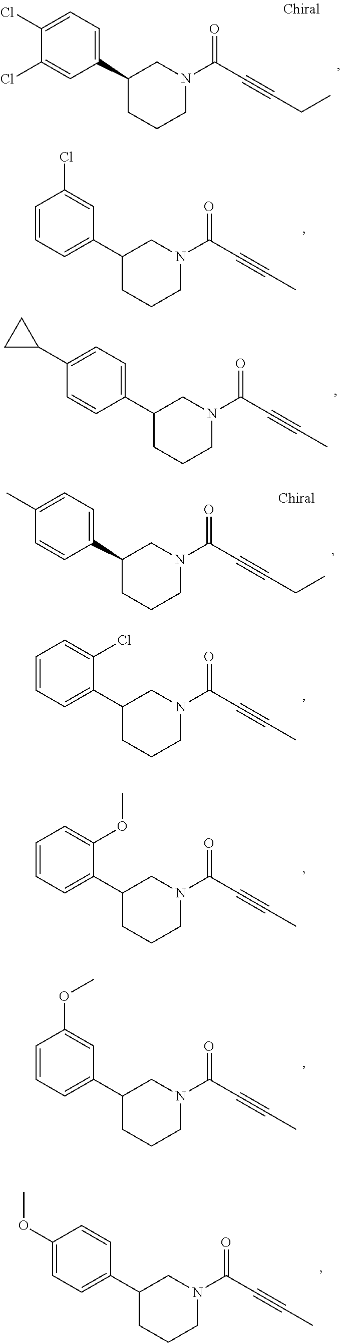 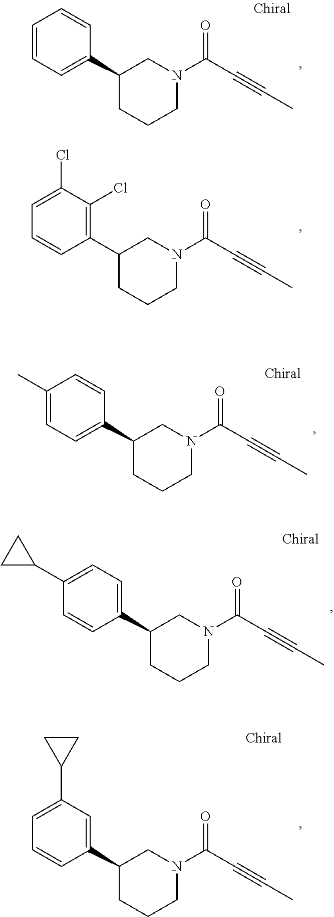 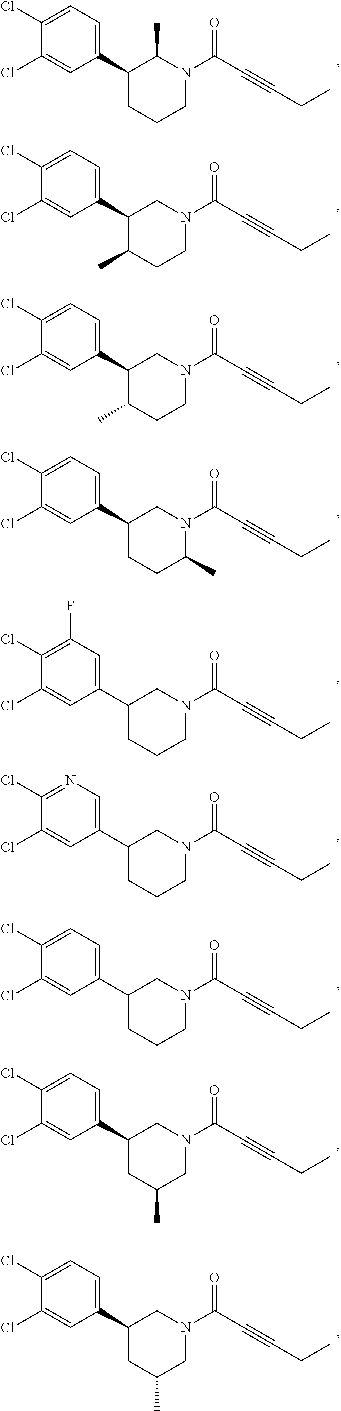 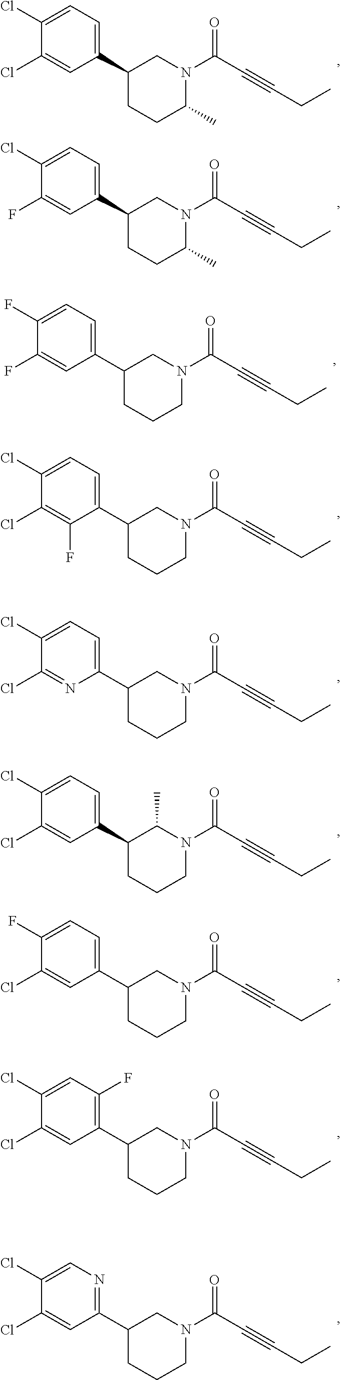 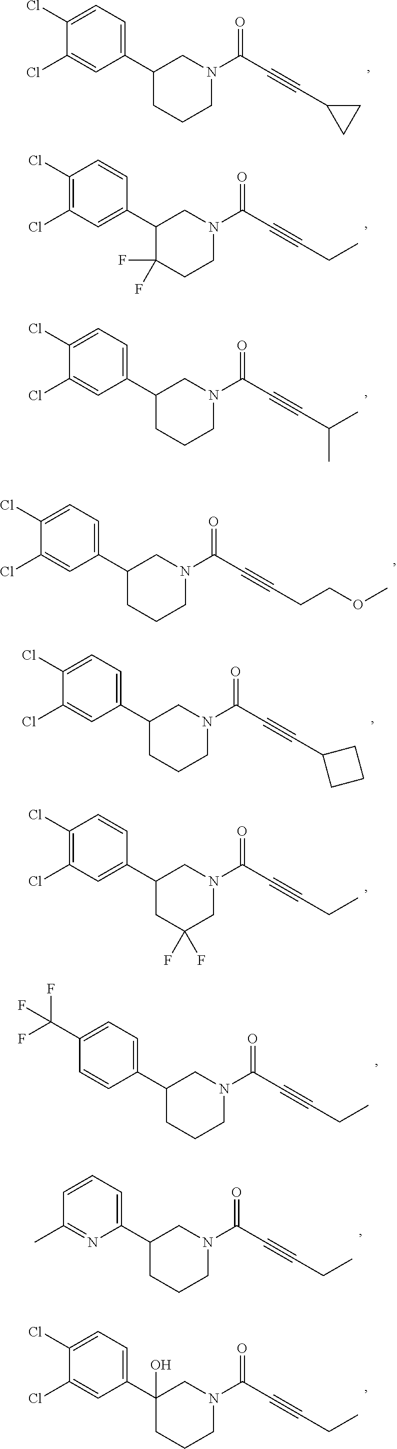 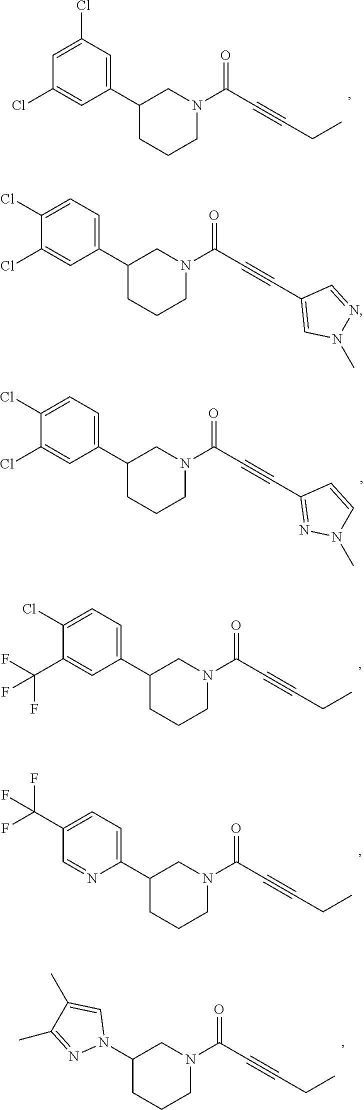 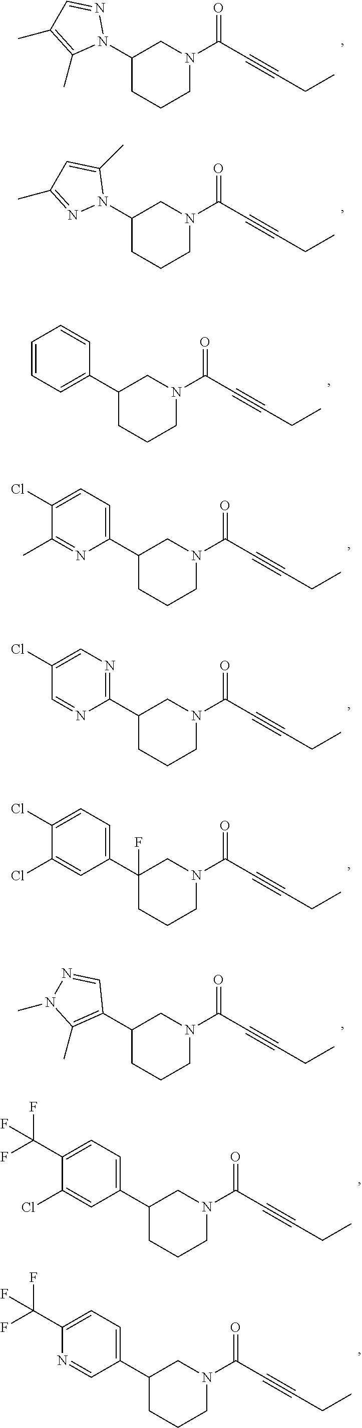 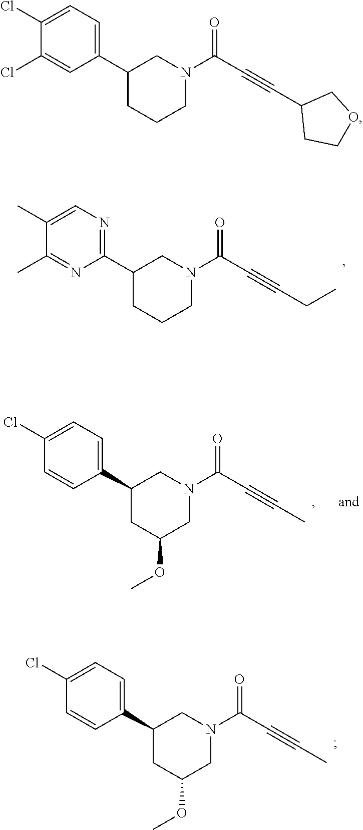 [SMILES: O=C(C#CC1CCOC1)N1CCCC(c2ccc(Cl)c(Cl)c2)C1 ; CCC#CC(=O)N1CCCC(c2ncc(C)c(C)n2)C1 ; CC#CC(=O)N1C[C@@H](OC)C[C@@H](c2ccc(Cl)cc2)C1 ; CC#CC(=O)N1C[C@H](OC)C[C@@H](c2ccc(Cl)cc2)C1]
or a pharmaceutically acceptable salt or solvate thereof.

14-18. (canceled)

19. A pharmaceutical composition comprising a compound of claim 1, or a pharmaceutically acceptable salt or solvate thereof, and a pharmaceutically acceptable carrier or excipient.

20. A method of inhibiting Janus kinase 1 (JAK1) in a subject in need of such inhibition, comprising administering to the subject a therapeutically effective amount a compound of claim 1, or a pharmaceutically acceptable salt or solvate thereof.

21. (canceled)

22. A method of treating a disease selected from the group consisting of an inflammatory disease, and an autoimmune disease in a subject in need of such treatment, comprising administering to the subject a therapeutically effective amount of a compound of claim 1, or a pharmaceutically acceptable salt or solvate thereof.

23. The method of claim 22, wherein the inflammatory or autoimmune disease is selected from the group consisting of Rheumatoid Arthritis (RA), Crohn's disease, ankylosing spondylitis (AS), psoriatic arthritis, psoriasis, ulcerative colitis, systemic lupus erythematosus (SLE), diabetic nephropathy, dry eye syndrome, Sjogren's Syndrome, alopecia areata, vitiligo, and atopic dermatitis.

24. A method of treating a disease selected from the group consisting of systemic inflammatory response syndrome, systemic onset juvenile rheumatoid arthritis, T-cell or FAB ALL, lymphoma, myeloma, leukemia, hematopoietic cancers, a diabetic condition such as insulin-dependent diabetes mellitus glaucoma, diabetic retinopathy or microangiopathy, chronic inflammation, glomerulonephritis, graft rejection, fibrosis, cirrhosis, thyroiditis, asthma, ulcerative colitis, inflammatory bowel disease, diabetes, diabetes mellitus, insulin dependent diabetes mellitus, allergic diseases, dermatitis scleroderma, graft versus host disease, organ transplant rejection (including bone marrow and solid organ rejection), acute or chronic immune disease associated with organ transplantation, alopecia, and alopecia areata, comprising administering to the subject a therapeutically effective amount of a compound of claim 1, or a pharmaceutically acceptable salt or solvate thereof.

25. (canceled)

26. (canceled)

27. A compound of Formula (III), or a pharmaceutically acceptable salt or solvate thereof: 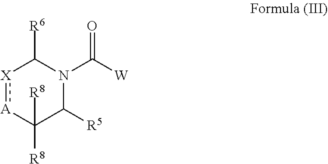
wherein: [symbol]
is a single or double bond;
X is C(J)(R⁷) when [symbol] is a single bond and C(J) when [symbol] is a double bond;
A is CR², NR¹, O or S or S(═O)₂ when [symbol] is a single bond; and CR when [symbol] is a double bond;
each R independently is H, halo, or C₁-C₆ alkyl;
R¹ is selected from the group C₁-C₆ alkyl, S(═O)₂—(C₁-C₆)alkyl, S(═O)₂—(C₆-C₁₀) aryl, C(═O)—(C₁-C₆) alkyl, or C(═O)—(C₆-C₁₀)aryl;
J is a C₆-C₁₀ aryl or a five- to six-membered heteroaryl, each of which is optionally substituted with 1-4 R³;
each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halo, C₁-C₆ haloalkyl, cyano and N(R⁴)₂; or two R³ optionally are taken together with the carbon atoms to which they are attached to form a five- or six-membered heterocycle or a C₃-C₆ cycloalkyl;
R⁵ is selected from the group consisting of hydrogen, C₁-C₆ alkyl, hydroxy, C₁-C₆ alkoxy, halogen, and —N(R³)₂;
R⁶ is H or C₁-C₆ alkyl;
R⁷ is H, hydroxy, or C₁-C₆ alkyl;
each R⁸ independently is H, halo, C₁-C₆ alkoxy, or C₁-C₆ alkyl;
n is 0, 1, or 2; and
W comprises an electrophile that reacts and forms a covalent bond with the sulfur atom at cysteine 817 as set forth in SEQ ID NO: 1, 2, 3, or 4.

28. The compound of claim 27 or a pharmaceutically acceptable salt or solvate thereof, wherein W comprises an alkynyl moiety.

29. (canceled)

30. The compound of claim 27, or a pharmaceutically acceptable salt or solvate thereof, wherein in Formula (III):
in J:
the optionally substituted C₆-C₁₀ aryl is phenyl which is optionally substituted with 1-4 R³;
the optionally substituted five- to six-membered heteroaryl is selected from the group consisting of pyridyl, pyrazolyl, and pyrimidinyl, each of which is optionally substituted with 1-4 R³;
in R¹:
the C₁-C₆ alkyl is methyl;
the C₁-C₆ alkyl in S(═O)₂—(C₁-C₆)alkyl is methyl;
the C₆-C₁₀ aryl in S(═O)₂—(C₆-C₁₀) aryl is phenyl;
the C₁-C₆ alkyl in C(═O)—(C₁-C₆) alkyl is methyl; and
the C₆-C₁₀ aryl in C(═O)—(C₆-C₁₀) aryl is phenyl;
in R³:
the C₁-C₆ alkyl is selected from the group consisting of methyl, and ethyl;
the C₃-C₆ cycloalkyl is cyclopropyl;
the C₁-C₆ alkoxy is methoxy; and
the C₁-C₆ haloalkyl is trifluoromethyl;
in R²:
the four- to six-membered heterocyclyl is selected from the group consisting of azetidinyl, tetrahydrofuranyl, piperidinyl, and pyrrolidinyl, each of which is optionally substituted with 1-3 R⁴;
the C₆-C₁₀ aryl is phenyl which is optionally substituted with 1-3 R⁴;
the C₃-C₈ cycloalkyl is selected from the group consisting of cyclopropyl, cyclobutyl, and cyclopentyl, each of which is optionally substituted with 1-3 R⁴;
the five- to six-membered heteroaryl is pyrazolyl which is optionally substituted with 1-3 R⁴;
the C₁-C₆ alkyl is selected from the group consisting of methyl, ethyl, and isopropyl, which is optionally substituted with 1-3 R⁴;
the C₁-C₆ alkyl-hydroxy is hydroxymethyl; and
the C₁-C₆ alkyl-amino is —CH₂—CH₂—NH—;
in R⁴:
the R^{4a} in S(═O)₂R^{4a} is selected from the group consisting of methyl and phenyl; and
the R^{4a} in C(═O)R^{4a} is methyl;
in R⁵:
the C₁-C₆ alkyl is methyl;
in R⁶:
the C₁-C₆ alkyl is methyl; and
in R⁸:
the C₁-C₆ alkyl is methyl; and
the C₁-C₆ alkoxy is methoxy.

31. (canceled)

32. (canceled)

33. The compound of claim 27, or a pharmaceutically acceptable salt or solvate thereof, wherein: A is CR², NR¹, or O.

34. The compound of claim 27, or a pharmaceutically acceptable salt or solvate thereof, wherein: n is 0, 1, or 2.

35-46. (canceled)

47. A pharmaceutical composition comprising a compound or a pharmaceutically acceptable salt or solvate thereof according to claim 27 and a pharmaceutically acceptable carrier or excipient.

48. A method of inhibiting JAK in a subject, comprising administering to the subject in need of such inhibition a therapeutically effective amount of a compound according to claim 27 or a pharmaceutically acceptable salt or solvate thereof.

49. A method of treating a disease mediated by JAK1 in a subject, comprising administering to the subject in need of such treatment a therapeutically effective amount of a compound according to claim 27 or a pharmaceutically acceptable salt or solvate thereof.

50-52. (canceled)

53. A method of inhibiting JAK1 comprising effecting a non-naturally occurring covalent modification at cysteine 817 as set forth in SEQ ID NO: 1, 2, 3, or 4; the modification resulting from a bond forming reaction between an electrophile and the cysteine 817 as set forth in SEQ ID NO: 1, 2, 3, or 4, wherein a sulfur atom at the cysteine residue undergoes a reaction with the electrophile.

54. A modified JAK1 protein comprising a non-naturally occurring small molecule fragment having a covalent bond to cysteine 817 of the JAK1 protein, wherein the modified JAK1 protein comprises SEQ ID NO: 1, 2, 3, or 4; and has the structure of Formula (IV): 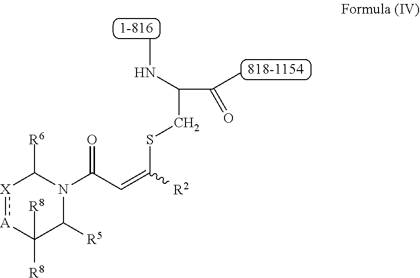
wherein in Formula (IV): [symbol]
is a single or double bond;
X is C(J)(R⁷) when [symbol] is a single bond and C(J) when [symbol] is a double bond;
A is CR², NR¹, O or S or S(═O)₂ when [symbol] is a single bond; and CR when [symbol] is a double bond;
each R independently is H, halo, or C₁-C₆ alkyl;
R¹ is selected from the group C₁-C₆ alkyl, S(═O)₂—(C₁-C₆)alkyl, S(═O)₂—(C₆-C₁₀) aryl, C(═O)—(C₁-C₆) alkyl, or C(═O)—(C₆-C₁₀)aryl;
J is a C₆-C₁₀ aryl or a five- to six-membered heteroaryl, each of which is optionally substituted with 1-4 R³;
each R³ is independently selected from the group consisting of C₁-C₆ alkyl, C₃-C₆ cycloalkyl, C₁-C₆ alkyl-hydroxy, C₁-C₆ alkyl-amino, C₁-C₆ alkoxy, hydroxyl, C₂-C₆ alkenyl, C₂-C₆ alkynyl, halo, C₁-C₆ haloalkyl, cyano and N(R⁴)₂; or two R³ optionally are taken together with the carbon atoms to which they are attached to form a five- or six-membered heterocycle or a C₃-C₆ cycloalkyl;
R² is selected from the group consisting of C₆-C₁₀ aryl, five- or six-membered heteroaryl, C₁-C₆ alkyl, C₃-C₈ cycloalkyl, C₁-C₆ alkyl-hydroxy, C₁-C₆ alkoxy, C₁-C₆ alkyl-amino, C₂-C₆ alkenyl, C₂-C₆ alkynyl, C₁-C₆ haloalkyl, or four- to six-membered heterocyclyl, each of which is optionally substituted with 1-3 R⁴;
each R⁴ is independently selected from the group consisting of hydroxyl, C₁-C₆ alkoxy, C₁-C₆ alkyl, C(═O)R^{4a} or S(═O)₂R^{4a} or wherein two R⁴ optionally are taken together with the carbon atoms to which they are attached to form a five- to six-membered heterocyclyl or a C₃-C₆ cycloalkyl, wherein each R^{4a} independently is hydrogen or C₁-C₆ alkyl;
R⁵ is selected from the group consisting of hydrogen, C₁-C₆ alkyl, hydroxy, C₁-C₆ alkoxy, halogen, and —N(R³)₂;
R⁶ is H or C₁-C₆ alkyl;
R⁷ is H, hydroxy, or C₁-C₆ alkyl;
each R⁸ independently is H, halo, C₁-C₆ alkoxy, or C₁-C₆ alkyl; and
n is 0, 1, or 2; and 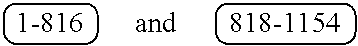
and represent amino acid sequences 1-816 and 818-1154 respectively in SEQ ID NO: 1, 2, 3, or 4.

55. The modified JAK1 protein of claim 54, wherein in Formula (IV):
in J:
the optionally substituted C₆-C₁₀ aryl is phenyl which is optionally substituted with 1-4 R³;
the optionally substituted five- to six-membered heteroaryl is selected from the group consisting of pyridyl, pyrazolyl, and pyrimidinyl, each of which is optionally substituted with 1-4 R³;
in R¹:
the C₁-C₆ alkyl is methyl;
the C₁-C₆ alkyl in S(═O)₂—(C₁-C₆)alkyl is methyl;
the C₆-C₁₀ aryl in S(═O)₂—(C₆-C₁₀) aryl is phenyl;
the C₁-C₆ alkyl in C(═O)—(C₁-C₆) alkyl is methyl; and
the C₆-C₁₀ aryl in C(═O)—(C₆-C₁₀) aryl is phenyl;
in R³:
the C₁-C₆ alkyl is selected from the group consisting of methyl, and ethyl;
the C₃-C₆ cycloalkyl is cyclopropyl;
the C₁-C₆ alkoxy is methoxy; and
the C₁-C₆ haloalkyl is trifluoromethyl;
in R²:
the four- to six-membered heterocyclyl is selected from the group consisting of azetidinyl, tetrahydrofuranyl, piperidinyl, and pyrrolidinyl, each of which is optionally substituted with 1-3 R⁴;
the C₆-C₁₀ aryl is phenyl which is optionally substituted with 1-3 R⁴;
the C₃-C₈ cycloalkyl is selected from the group consisting of cyclopropyl, cyclobutyl, and cyclopentyl, each of which is optionally substituted with 1-3 R⁴;
the five- to six-membered heteroaryl is pyrazolyl which is optionally substituted with 1-3 R⁴;
the C₁-C₆ alkyl is selected from the group consisting of methyl, ethyl, and isopropyl, which is optionally substituted with 1-3 R⁴;
the C₁-C₆ alkyl-hydroxy is hydroxymethyl; and
the C₁-C₆ alkyl-amino is —CH₂—CH₂—NH—;
in R⁴:
the R^{4a} in S(═O)₂R^{4a} is selected from the group consisting of methyl and phenyl; and
the R^{4a} in C(═O)R^{4a} is methyl;
in R⁵:
the C₁-C₆ alkyl is methyl;
in R⁶:
the C₁-C₆ alkyl is methyl; and
in R⁸:
the C₁-C₆ alkyl is methyl; and
the C₁-C₆ alkoxy is methoxy.

56-71. (canceled)
